(19) United States
(12) Patent Application Publication
(10) Pub. No.: US 2023/0201265 A1
(43) Pub. Date: Jun. 29, 2023

(54) COMPOSITIONS COMPRISING NON-VIABLE FECAL MICROBIOTA AND METHODS OF USE THEREOF

(71) Applicant: Finch Therapeutics Holdings LLC, Somerville, MA (US)

(72) Inventors: Mark SMITH, Cambridge, MA (US); Christopher WEIDENMAIER, Brookline, MA (US); Sonia TIMBERLAKE, Brookline, MA (US)

(73) Assignee: Finch Therapeutics Holdings LLC, Somerville, MA (US)

(21) Appl. No.: 17/915,765

(22) PCT Filed: Mar. 31, 2021
(86) PCT No.: PCT/US2021/025266

§ 371 Date: Sep. 29, 2022

Related U.S. Application Data
(60) Provisional application No. 63/035,386, filed on Jun. 5, 2020.
(60) Provisional application No. 63/016,725, filed on Apr. 28, 2020.
(60) Provisional application No. 63/008,467, filed on Apr. 10, 2020.
(60) Provisional application No. 63/002,975, filed on Mar. 31, 2020.

(51) Int. Cl. A61K 35/24; C12N 1/36; G01N 33/569; A61K 35/741; A61K 39/395; A61K 39/35; A61K 41/10; A61P 35/00; A61P 37/08; A61P 11/00

(52) U.S. Cl. CPC A61K 35/24; A61K 35/741; A61K 39/35; A61K 39/3955; A61K 41/10; A61P 11/00; A61P 35/00; A61P 37/08; C12N 1/36; G01N 33/56983; G01N 2469/10; G01N 2470/04

(57) ABSTRACT

The present disclosure relates to compositions comprising a non-viable fecal bacterial preparation for treating various gastrointestinal-related conditions and disorders. The present disclosure also relates to stool-based compositions comprising adjuvants to facilitate the efficacy of immunomodulators such as vaccines and immunotherapies.

### CROSS-REFERENCE TO RELATED APPLICATIONS

This application claims priority to U.S. Provisional Application No. 63/002,975, filed Mar. 31, 2020, U.S. Provisional Application No. 63/008,467, filed Apr. 10, 2020, U.S. Provisional Application No. 63/016,725, filed Apr. 28, 2020. This application also claims priority to U.S. Provisional Application No. 63/035,386, filed Jun. 5, 2020. The U.S. Provisional Applications are incorporated by reference herein in their entireties.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenized stool, or cultured stool components such as Clostridia, to implant in the colon and thereby displace or eradicate pathogenic bacteria, e.g., C. difficile. Fecal bacteriotherapy has also been successful in treating conditions having a neurological component, such as ASD, Parkinson’s Disease, and Multiple Sclerosis and Chronic Fatigue Syndrome.

To ensure the safety of human stool donor-derived products, both the human stool donors and their stool samples are screened and tested through rigorous processes to exclude potentially transmittable diseases. See Cammarota G, Ianiro G, Kelly CR, et al., International consensus conference on stool banking for faecal microbiota transplantation in clinical practice, Gut 2019, 68:2111-2121. In certain cases, administration of an FMT can cause infection in a patient, for example due to the presence of multi-drug resistant bacteria in donor stool.

In at least some cases, the precise mechanism by which fecal bacteriotherapy acts to treat a condition, for example an autoimmune condition, is unknown. For example, a typical donor fecal microbiota administered to a patient during fecal bacteriotherapy can contain hundreds of bacterial strains, and the identity of the strains necessary for the treatment of an autoimmune disorder, as well as the mechanisms by which such introduced strains interact with each other and the bacteria of the patient’s endogenous microbiome, is largely unknown. Further, potential variation in (i) the identity and relative abundance of particular bacterial strains across different donor samples; and (ii) the degree to which a particular bacterial strain engrafts in the intestine of a bacteriotherapy recipient, can lead to uncertainty regarding efficacy of fecal bacteriotherapy across a patient group afflicted with or susceptible to a disorder.

### SUMMARY

It has been surprisingly discovered that a product derived from a human donor’s stool which has been treated to neutralized or kill or remove viable cells may be useful as a pharmaceutical composition to treat or prevent various diseases or conditions. In certain aspects, such stool comprises one or more antigens of a human pathogen (e.g., virus) which may be useful in a pharmaceutical composition (e.g. a vaccine) to prevent or treat various diseases and related conditions, including viral infections. In some aspects, contrary to prevailing guidelines for the manufacture and use of products derived from human stool donors, the product and methods described herein comprise the use of stool of a human donor who is or has been infected with one or more pathogens (e.g., viruses).

The present disclosure provides for compositions and methods for treating various gastrointestinal-related conditions and disorders with non-viable microbes derived or extracted from stool of a human donor.

In an aspect, the present disclosure provides a pharmaceutical composition comprising a fecal bacterial preparation comprising fecal bacteria derived from a stool of a human, wherein the fecal bacteria are not viable.

In another aspect, the present disclosure provides a pharmaceutical composition comprising a microbial preparation comprising microbes derived from a stool of a human donor, wherein the microbes are not viable.

In another aspect, the present disclosure provides a pharmaceutical composition comprising a sterile fecal microbiota, wherein the fecal microbiota is extracted from a stool of a human donor.

In another aspect, the present disclosure provides a method of treating a disorder, the method comprising administering to a subject in need thereof a pharmaceutical composition comprising a fecal bacterial preparation, wherein the fecal bacterial preparation does not contain viable bacteria.

In a further aspect, the present disclosure provides a method of treating a disorder in a subject in need thereof, the subject having small intestinal bacterial overgrowth (SIBO), the method comprising administering to the subject a pharmaceutical composition comprising a sterile fecal microbiota.

In yet another aspect, the present disclosure provides a method comprising: receiving a stool or portion thereof from a human donor, wherein the stool comprises a fecal microbiota; sterilizing the fecal microbiota; and incorporating the sterilized fecal microbiota into a pharmaceutical composition.

In another aspect, the present disclosure provides a method of stimulating the immune system in a subject in need thereof, the method comprising administering to the subject a pharmaceutical composition comprising an adjuvant comprising a fecal bacterial preparation, wherein the fecal bacterial preparation comprises fecal bacteria from a stool of a human donor.

In another aspect, the present disclosure provides a pharmaceutical composition for adjuvant therapy comprising anon-viable fecal bacterial preparation.

### BRIEF DESCRIPTION OF DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 illustrates a sandwich ELISA.

FIG. 2 illustrates an exemplary microtiter plate setup according to an assay provided in this disclosure.

FIG. 3 illustrates an alternative detection assay for SARS-CoV-2 according to Example 4.

FIG. 4 is a schematic illustration showing the immune response induced in a subject following administration of a composition comprising microbial antigen and adjuvant.

FIG. 5 is a flowchart showing exemplary steps for manufacturing a composition described herein.

FIG. 6A is a graph showing the amount of SARS-CoV-2-directed antibody detected from convalescent serum of COVID-19 survivors by unpasteurized and pasteurized viral proteins immobilized on a microplate.

FIG. 6B is a graph plotting the dataset from FIG. 6A to show the measured difference between unpasteurized and pasteurized proteins at each concentration computed as the percentage of epitopes retained after pasteurization.

### DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. By way of example, “an element” means at least one element and can include more than one element.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

To avoid any doubt, used herein, terms or phrases such as “about”, “at least”, “at least about”, “at most”, “less than”, “greater than”, “within” or alike, when followed by a series of list of numbers of percentages, such terms or phrases are deemed to modify each and every number of percentage in the series or list, regardless whether the adverb, preposition, or other modifier phrase is reproduced prior to each and every member.

As used herein, the term “relative abundance” refers to relative representation of an organism of a particular kind (e.g., a bacterial strain, species, or genus) relative to all organisms of similar nature in a certain community (e.g., a preparation of fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing as described in Gevers et al., “The treatment-naive microbiomes in new-onset Crohn’s disease.” Cell Host & Microbe, 15(3):382-92(2014). In an aspect, high-throughput sequencing is based on 16S rRNA gene sequencing. In another aspect, high-throughput sequencing is based on whole-genome short-gun metagenomic sequencing. Unless specified otherwise, a bacterial relative abundance mentioned herein is measured via high-throughput sequencing of 16S rRNA targeting the V4 variable region as described in Gevers et al., Cell Host & Microbe, 15(3):382-92(2014). In a further aspect, propidium monoazide (PMA) is used to differentiate between viable and dead fecal microbes as shown in Chu et al., PLoS One 12(1): 1-16 (2017).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, a “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably.

Non-limiting examples of disorders, diseases, conditions or indications contemplated herein as targets of pharmaceutical compositions comprising a non-viable fecal bacterial preparation include acne, AIDS enteropathy, AIDS-related gastroenteritis, alopecia totalis, Alzheimer’s Disease, amyloidosis, amyotrophic lateral sclerosis, ankylosing spondylitis, anorexia, antibiotic associated colitis, Asperger’s syndrome, attention deficit disorder (ADD), attention deficit hyperactivity disorder (ADHD), autism spectrum disorder (ASD), Behcet’s Syndrome, chronic Clostridium difficile infection (CDI), chronic constipation, chronic depression, chronic fatigue syndrome (CFS), chronic idiopathic pseudo obstructive syndrome, chronic inflammation demyelinating polyneuropathy, chronic nausea, chronic urticaria, coeliac disease, collagenous colitis, colonic polyps, constipation predominant FBD, Crohn’s disease, cryptogenic cirrhosis, cyclic vomiting, dermatitis herpetiformis, diabetes, familial mediterranean fever, fatty liver, functional bowel disease (FBD), gastro-oesophageal reflux, gillian-barre syndrome, glomerulonephritis, haemolytic uraemic syndrome, halitosis, IBS constipation-predominant, IBS diarrhea/constipation alternating, IBS diarrhea-predominant, IBS pain-predominant, idiopathic thrombocytopenic purpura (ITP), idiopathic/simple constipation, indeterminate colitis, inflammatory bowel disease (IBD), irritable bowel syndrome (IBS), juvenile diabetes mellitus, lyme disease, manic depressive illness, metabolic syndrome, microscopic colitis, migraine, mixed cryoglobulinaemia, mucous colitis, multiple sclerosis, myasthenia gravis, NASH (nonalcoholic steatohepatitis), non-rheumatoid arthritis, non-rheumatoid factor positive arthritis, non-ulcer dyspepsia, norwalk viral gastroenteritis, obesity, obsessive compulsive disorder, pain predominant FBD, Parkinson’s disease, polyarteritis, polyposis coli, primary biliary cirrhosis, primary Clostridium difficile infection (CDI), primary sclerosing cholangitis (PSC), pseudomembranous colitis, psychotic disorders, reiter’s syndrome, relapsing diverticulitis, Rett syndrome, rheumatoid arthritis, rosacea, rotavirus gastroenteritis, sacroiliitis, schizophrenia, scleroderma, Sjogren’s Syndome, small bowel bacterial overgrowth, sudden infant death syndrome (SIDS), systemic lupus erythematosus, ulcerative colitis, upper abdominal functional bowel disorder (FBD), vasculitic disorders, viral gastroenteritis, pre-diabetic syndrome, type I diabetes, type II diabetes, depression, schizophrenia, a mood disorder, vancomycin-resistant Enterococci (VRE) infection, methicillin-resistant Staphylococcus Aureus (MRSA) infection, an autoimmune disorder, an infection, an allergy or atopy and a neurological disorder.

In an aspect, allergy indications contemplated herein as targets of pharmaceutical compositions comprising a non-viable fecal bacterial preparation include food allergies including peanuts, soybeans, cow’s milk, milk protein, tree nuts, shellfish, fish, wheat, eggs, sesame, dairy; seasonal allergies; outdoor allergies including grass and trees; animal or pet allergies; insect allergies; and mold allergies.

In certain aspects, a subject administered a composition described herein has or is at risk of developing small intestinal bacterial overgrowth (SIBO).

In other aspects, a subject administered a composition described herein has cancer or is being treated with a cancer therapy. In another aspect, a composition described herein is used as a treatment prior to a cancer therapy, with a cancer therapy, or after a cancer therapy.

As used herein, a “microbiota” and “flora” refer to a community of microbes that live in or on a subject’s body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject’s feces. A pharmaceutical composition described herein comprising a non-viable microbiota preparation can be prepared by treating such a fecal microbiota with one or more sterilizing agents and incorporating the non-viable microbiota or portion thereof into the composition. A fecal microbiota preparation includes fecal bacteria. Herein a “fecal bacterial preparation” or a “preparation of fecal bacteria” refers to a composition that comprises multiple strains of bacteria and optionally non-bacterial microbes that have been extracted or harvested together from a stool of a donor. Following extraction or harvesting from a stool, the multiple strains of bacteria are not isolated or purified away from each other (although they may have been co-cultured) and hence each strain in a fecal bacterial preparation traces a temporally continuous co-habitation or co-environment with every other strain in the preparation, from their presence together in a stool to their use in manufacturing a pharmaceutical composition described herein comprising a non-viable fecal bacterial preparation. In one aspect, a fecal bacterial preparation comprises a complete or substantially complete fecal microbiota derived from a human donor.

In an aspect, a fecal bacterial preparation comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least forty, at least fifty, at least sixty, at least seventy, at least eighty, at least ninety, at least one hundred, at least one hundred fifty, at least two hundred, at least two hundred fifty, at least three hundred, at least three hundred fifty, at least four hundred, at least four hundred fifty, at least five hundred, at least six hundred, at least seven hundred, at least eight hundred, at least nine hundred, at least one thousand, or more than one thousand strains of fecal bacteria. In an aspect, a fecal bacterial preparation comprises strains of bacteria from different species. In an aspect, a fecal bacterial preparation comprises strains of bacteria from different genera. In an aspect, a fecal bacterial preparation comprises strains of bacteria from different families. In an aspect, a fecal bacterial preparation comprises strains of bacteria from different orders. In an aspect, a fecal bacterial preparation comprises strains of bacteria from different classes. In an aspect, a fecal bacterial preparation comprises strains of bacteria from different phyla. In an aspect, a fecal bacterial preparation comprises the complement of bacteria in a fecal microbiota. In an aspect, a fecal bacterial preparation is in the form of a substantially complete fecal microbiota. Herein the term “substantially complete fecal microbiota” refers to a preparation that comprises viable bacterial cells from all or substantially all of the bacterial taxa represented among the viable bacterial cells in the stool from which the fecal microbiota was extracted. In an aspect, the relative abundance of viable bacterial cells from at least two of the taxa in the substantially complete fecal microbiota is proportional to the relative abundance of the viable cells from those taxa in the stool from which the fecal microbiota was extracted.

Typically, a fecal bacterial preparation described herein is extracted from a stool or stools of a single human donor. In an aspect, a fecal bacterial preparation from the stool of one donor is combined or blended with a fecal bacterial preparation from the stool of a different donor to produce a composition comprising consortia of fecal bacteria.

In an aspect, a fecal bacterial preparation comprises bacteria that have not been subject to culturing and as such comprise uncultured fecal bacteria. Herein “uncultured fecal bacteria” or a “preparation of uncultured fecal bacteria” refers to a mixture of bacteria extracted together from a stool, wherein the bacteria in the mixture have not been subjected to culturing or fermentation in artificial growth media. Herein such a preparation of uncultured fecal bacteria can also be referred to as a collection of uncultured fecal bacteria or a population or community of uncultured fecal bacteria.

In another aspect, a fecal bacterial preparation comprises bacterial strains that have been subjected to culturing or fermentation. For example, a fecal microbiota can be mixed with a culturing or fermentation medium that facilitates the replication of bacterial cells of different strains and optionally the maintenance over time during replication of the relative abundance of particular strains relative to the relative abundance of such strains in the fecal microbiota prior to culturing or fermentation.

In some aspects, a fecal bacterial preparation comprises non-selected fecal bacteria. Herein “non-selected fecal bacteria” refers to a population or community of fecal bacterial strains (e.g., present in a fecal microbiota) extracted from one or more stool samples without subjecting the extracted population or community to environmental conditions that intentionally select for a particular type, state or taxonomic category of bacteria (e.g., by deliberate removal of certain strains of bacteria, treatment of the population or community with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract a population or community of fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a non-selected fecal bacterial preparation comprises all or substantially all of the bacteria of a fecal microbiota of a stool sample. In certain aspects, a non-selected fecal bacterial preparation comprises all or substantially all of the taxonomic strains of a fecal microbiota of a stool or portion thereof. In certain aspects, a non-selected fecal bacterial preparation comprises all or substantially all of the species of a fecal microbiota of a stool or portion thereof. In certain aspects, a non-selected fecal bacterial preparation comprises all or substantially all of the genera of a fecal microbiota of a stool or portion thereof. In certain aspects, a fecal bacterial preparation comprises all or substantially all of the phyla of a fecal microbiota of a stool sample. Therefore, the bacterial structure of a non-selective fecal bacterial preparation can substantially resemble microbial constituents and the bacterial population or community structure found in such fecal sample.

In an aspect, a fecal bacterial preparation comprises a bacterial population or community that comprises at least 2, 5, 10, 20, 30, 40, 50, 100, 200, 300, 400, 500, or 600 bacterial species or strains. In another aspect, a fecal bacterial preparation comprises a bacterial population or community that comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a fecal bacterial preparation and/or non-selected fecal bacteria does not comprise an antibiotic resistant population of bacteria.

In another aspect, manufacture of a non-viable fecal bacterial preparation can involve steps that select for a particular, type, state, or taxonomic category of bacteria (e.g., by deliberate removal of certain strains of bacteria and/or treatment of the population with a selective agent such as ethanol or chloroform). In certain aspects, such a preparation can be combined with one or more bacterial isolates (e.g., viable or non-viable) to form a bacterial mixture for incorporation into a pharmaceutical composition.

Herein a fecal bacterial preparation is distinguished from a single, purified strain of bacteria such as a bacterial isolate. As used herein, “bacterial isolate” refers to an isolated group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. In the bacterial context, the term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria, but in certain aspects can comprise a pathogen, e.g., a pathogen treated (for example with heat) to remove viability but retain immunogenicity when administered to a subject. In an aspect, a bacterial isolate can be a probiotic, or an ingredient in a probiotic. The present disclosure contemplates that a pharmaceutical composition can comprise a bacterial mixture comprising a bacterial isolate in combination with a fecal bacterial preparation lacking viable bacteria.

As used herein, the term “bacterial cocktail”, sometimes called a “bacterial consortium”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising bacterial cells, which in some aspects can include one or more non-pathogenic bacterial isolates and/or a non-viable fecal bacterial preparation. In some aspects, a bacterial mixture comprises one or more non-pathogenic bacterial isolates. In some aspects, a bacterial mixture comprises a non-viable fecal bacterial preparation. In some aspects, a bacterial mixture comprises both of one or more non-pathogenic bacterial isolates and a non-viable fecal bacterial preparation.

As used herein, “fungal isolate” refers to an isolated group of substantially genetically identical fungal cells generated by proliferation via binary fission from a single predecessor fungal cell (e.g., by culturing the fungi). Typically, a fungal isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a fungal isolate can be grown as a pure culture of cells; in other aspects, multiple fungal isolates can be grown simultaneously in the same vessel as a mixed culture. In the fungal context, the term “substantially genetically identical” refers to the very high (e.g. >99.9%) genetic identity shared by different cells in uncontaminated pure compositions of fungal isolates, owing to their proliferation from a common predecessor, but accounts for minor genetic dissimilarity between cells due to accumulations of relatively rare mutations. Generally, a fungal isolate is synonymous with a pure culture of fungal cells. Typically, herein a fungal isolate consists of non-pathogenic fungi. In an aspect, a fungal isolate can be a probiotic, or an ingredient in a probiotic.

Any aspect described herein referencing or related to a bacterial isolate can be equally applicable to a fungal isolate. For example, all the disclosure and description herein about a mixture comprising a fecal bacterial preparation enriched, supplemented or “spiked” with one or more bacterial isolates applies equally to a mixture comprising a fecal bacterial preparation enriched, supplemented or “spiked” with one or more fungal isolates. Further for example, all the disclosure and description herein about a mixture comprising a fecal bacterial preparation enriched, supplemented or “spiked” with one or more bacterial isolates applies equally to a mixture comprising a fecal bacterial preparation enriched, supplemented or “spiked” with one or more bacterial isolates and one or more fungal isolates.

As used herein, “archaeal isolate” refers to an isolated group of substantially genetically identical archaeal cells generated by proliferation via binary fission from a single predecessor archaeal cell (e.g., by culturing the archaea). Typically, an archaeal isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, an archaeal isolate can be grown as a pure culture of cells; in other aspects, multiple archaeal isolates can be grown simultaneously in the same vessel as a mixed culture. In the archaeal context, the term “substantially genetically identical” refers to the very high (e.g. >99.9%) genetic identity shared by different cells in uncontaminated pure compositions of archaeal isolates, owing to their proliferation from a common predecessor, but accounts for minor genetic dissimilarity between cells due to accumulations of relatively rare mutations. Generally, an archaeal isolate is synonymous with a pure culture of archaeal cells. Typically, herein an archaeal isolate consists of non-pathogenic archaea. In an aspect, an archaeal isolate can be a probiotic, or an ingredient in a probiotic.

Any aspect described herein referencing or related to a bacterial isolate can also be equally applicable to an archaeal isolate. For example, all the disclosure and description herein about a mixture comprising a fecal bacterial preparation enriched, supplemented or “spiked” with one or more bacterial isolates applies equally to a mixture comprising a fecal bacterial preparation enriched, supplemented or “spiked” with one or more archaeal isolates (and/or one or more fungal isolates). Further for example, all the disclosure and description herein about a mixture comprising a fecal bacterial preparation enriched, supplemented or “spiked” with one or more bacterial isolates applies equally to a mixture comprising a fecal bacterial preparation enriched, supplemented or “spiked” with one or more bacterial isolates and one or more archaeal isolates. Similarly, all the disclosure and description herein about a mixture comprising a fecal bacterial preparation enriched, supplemented or “spiked” with one or more bacterial isolates applies equally to a mixture comprising a fecal bacterial preparation enriched, supplemented or “spiked” with one or more bacterial isolates, one or more archaeal isolates, and one or more archaeal isolates.

As used herein, “therapeutically effective amount,” “effective amount” or “pharmaceutically active dose” refers to an amount of a composition comprising an antigen of a microbe (e.g. pathogen) which is effective to vaccinate a subject against a virus and/or induce an immune response comprising the generation of antibodies that target or recognize the microbial antigen. In another aspect, “therapeutically effective amount,” “effective amount” or “pharmaceutically active dose” refers to an amount of a composition which is effective in treating the named disease, disorder, condition, or symptom.

As used herein, “isolated” or “purified” refers to a bacterium or other entity or substance that has been (1) separated from at least some of the components with which it was associated when initially produced (whether it was initially produced in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

Herein a “fecal filtrate” refers to any preparation manufactured by filtering a stool or stool homogenate obtained from one or more stool donors (e.g., human donors). The filtering step to produce a fecal filtrate can remove non-living rough particulate matter and/or fiber from the stool but leave substantially intact the fecal microbiota of the stool (e.g., comprising microbial cells such as bacterial and fungal cells). In an aspect, a stool (or stool homogenate) can be filtered using a filter having a pore size of no greater than 500 µm, 450 µm, 400 µm, 350 µm, 300 µm, 250 µm, 200 µm, 150 µm, 100 µm, 50 µm, 25 µm, 10 µm, 5 µm, or 1 µm.

As used herein, the terms “non-pathogenic” in reference to a bacterium or any other organism or entity includes any such organism or entity that does not cause or exacerbate the severity of a disease, disorder or condition of a host organism containing the bacterium, organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (CFUs) refers to an estimate of the number of viable microorganism cells in a given sample. The number of CFUs can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be non-viable (i.e., dead or dying), whereas cells with an intact membrane are considered live. For example, SYTO 9 and propidium iodide are used to stain and differentiate live and dead bacteria. See Stocks, Cytometry A. 2004 Oct;61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 Oct; 80(19): 5884-5891. A “non-viable” cell refers to a cell or group of cells that is incapable of dividing. Such a cell may be “dead” in that the cell no longer executes or is capable of executing cellular processes indicative of life, such as production or storage of energy, protein synthesis, transcription, translation or intramembrane transport. However, “non-viable” also refers to a cell that is permanently incapable of dividing yet continues to demonstrate one or more basic cellular processes. A spore capable of germinating and dividing is not a “non-viable” organism as contemplated herein. As used herein a “sterile” preparation refers to a preparation that contains no viable cells (i.e., consists of non-viable cells). A “sterile fecal microbiota” or “sterile fecal filtrate” refers to a fecal microbiota or fecal filtrate that contains no viable cells. A “sterilizing agent” refers to any agent that may be applied to a preparation of viable cells to destroy viability all of the cells and thereby produce a preparation consisting only of non-viable cells. Examples of sterilizing agents include heat, steam, filtration, various antibiotics (applied to bacteria), sonication, radiation and pressure.

A “non-viable fecal bacterial preparation” refers to a fecal bacterial preparation that has been treated so as to lack viable bacteria. In certain aspects, a non-viable bacterial preparation comprises one or more non-viable bacterial cells. In other aspects, a non-viable bacterial preparation does not comprise any intact bacterial cells (viable or non-viable), but instead contains lysed or ruptured bacterial cells and the contents thereof, for example in addition to non-bacterial components.

As used herein, “Shannon Diversity Index” refers to a diversity index that accounts for abundance and evenness of species present in a given community using the formula

$$\text{H =} - {\sum }_{i = 1}^{R} {p}_{i} \text{ln} {p}_{i} ,$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

As used herein, “antibiotic” refers to a substance that is used to treat and/or prevent bacterial infection by killing bacteria, inhibiting the growth of bacteria, or reducing the viability of bacteria.

Described herein are pharmaceutical compositions comprising a non-viable fecal bacterial preparation. In an aspect, the non-viable fecal bacterial preparation is prepared from a composition comprising non-selected fecal bacteria. In an aspect, the non-viable fecal bacterial preparation comprises non-bacterial microbes. For example, the non-viable fecal bacterial preparation can comprise a fecal microbiota or a substantially complete fecal microbiota that contains no viable bacteria. In certain aspects, a non-viable fecal bacterial preparation may comprise viable non-bacterial cells, e.g., archaea or fungi from a fecal microbiota. In other aspects, a non-viable fecal bacterial preparation does not contain viable microbes of any kind.

A fecal bacterial preparation can comprise any fecal material from a stool of a donor (e.g., human donor). Herein “fecal material” can refer to any substance or substances present in a stool as the stool emerges from a donor. Non-limiting examples of fecal material include a fecal microbiota or portion thereof (e.g., comprising bacteria, archaea, viruses, and/or fungi), one or more biological compounds (e.g., SCFA or bile acid) generated or released by a microbe of the fecal or intestinal microbiota or the stool donor, fiber, and rough particulate matter. In an aspect, a stool of a donor is subject to processing in order to manufacture a preparation comprising fecal material or a fecal bacterial preparation. For example, a stool can be homogenized and/or filtered to manufacture a fecal bacterial preparation (e.g., in a fecal filtrate) that following inactivation (e.g., by heat or sonication) can be incorporated into a pharmaceutical composition. In some aspects, a fecal filtrate comprises filtered and homogenized stool, if the stool is homogenized prior to filtering, for example in a saline buffer comprising a cryoprotectant. In another aspect, the filtering step can remove some or substantially all of the cells of the stool (e.g., including human cells or microbial cells such as fungal or bacterial cells) to produce sterile fecal material. In an example, a sterile fecal filtrate can be produced by subjecting stool of a donor to “sterile filtration” (e.g., using a syringe or bottle-top filter) that passes the stool through a filter or sieve comprising pores of no greater than 0.3 µm, no greater than 0.25 µm, no greater than 0.24 µm, no greater than 0.22 µm, no greater than 0.20 µm, no greater than 0.10 µm, or no greater than 0.1 µm in size to produce the sterile fecal filtrate. In a further aspect, fecal material (e.g. fecal filtrate) can be devoid of live cells as well as active viruses, for example by passing stool, a stool homogenate, or a fecal filtrate through a filter or sieve having pore sizes of no greater than 5 nm, 4 nm, 3 nm, 2 nm, 1 nm or 0.5 nm in size.

Any method, means or process known in the art can be used to generate a non-viable fecal bacterial preparation. In an aspect, a non-viable fecal bacterial preparation is manufactured from a fecal microbiota by treating the microbiota with heat. In an aspect, such heat treatment comprises a pasteurization step or an autoclaving step. For example, a non-viable fecal bacterial preparation can be generated by pasteurizing the microbiota (e.g., present in a stool, filtered and homogenized stool or a fecal filtrate) at 60° C. for approximately 10 hours. In other examples, a non-viable fecal bacterial preparation can be generated by heating the fecal microbiota to at least 40° C., at least 45° C., at least 50° C., at least 55° C., at least 60° C., at least 65° C., at least 70° C., at least 75° C., at least 80° C., at least 85° C., at last 90° C., at least 95° C., at least 100° C., at least 100° C., at least 110° C., at least 120° C. (e.g., in an autoclave), or greater than 120° C. In non-limiting examples, such heating can be applied for at least 1 minute, at least 5 minutes, at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 15 hours, at least 20 hours, or at least 24 hours. In certain aspects, the microbiota can be heated under pressure (e.g., in an autoclave). Heating can be by any known means including by hot water (e.g., water bath) or steam or use of a plate heat exchanger. Methods of generating a non-viable fecal bacterial preparation that can be employed with the methods described herein include treatment of the microbiota (e.g., present in a stool or fecal filtrate) with an antibiotic, heat, pressure (e.g., Pascalization), pulsed electric field electroporation, nonthermal plasma, sonication, radiation (e.g., with UV light, beta particles, gamma rays, or a combination thereof), solvent and/or detergent (e.g.,Triton X-100), with low pH (e.g., less than or equal to pH 4), or any combination of such methods. Steps (e.g., heating) to generate a non-viable fecal bacterial preparation can be performed on viable microbes present in any environmental context or state of purification. For example, non-limiting examples of substrates that can be heated and/or pasteurized to generate a non-viable fecal bacterial preparation include a fresh stool, a frozen stool, a fecal microbiota extracted from a stool, and/or a fecal filtrate.

In aspects, a non-viable fecal bacterial preparation incorporated into a pharmaceutical composition comprises bacterial cells that are ruptured, leaky, broken open, or porous such that normally internal contents of the cells (e.g., cytoplasm, protein, DNA, RNA, carbohydrates, metabolites, etc.) may be present in the preparation outside of the cells. Such rupturing to release the contents of the cells can be due to the process used to generate the non-viable preparation from a preparation of viable fecal bacteria (e.g., due to the application of a sterilizing agent to viable cells). In an aspect, sonication is employed (e.g., in combination with other sterilizing measures such as heat) to break open cells.

Incorporating ruptured bacterial cells or components thereof into a composition can advantageously enrich for molecules and subcellular structures capable of interacting with host intestinal epithelial cells to induce signaling pathways that are therapeutic in certain disease contexts. Non-limiting examples of cellular substances that can be enriched in a pharmaceutical composition include a peptide, a lipopolysaccharide, flagellin, a peptidoglycan, a lipoteichoic acid, a lipoprotein, or outer membrane vesicles. In certain aspects, the cellular substances enriched in the composition comprise molecules recognized by a pattern recognition receptor (PRR) of a host cell. In one aspect, contents of one or more immunomodulatory bacterial cells can act directly on a host cell of a subject via, for example, microbe-associated molecular patterns (MAMPS) associated with for example a membrane vesicle of the bacterial cell(s) or the surface of the non-viable bacterial cell(s). Such MAMPS play a major role in host immune responses to particular bacterial species. MAMPS are sensed by PRRs expressed on most host cell types that are in contact with bacteria. Examples of MAMPS of bacteria described herein include unmethylated 2′-deoxyribo(cytidine-phosphate-guanine) (CpG) dinucleotides, bacterial peptidoglycans, bacterial lipopolysaccharides (LPS, which interacts with co-receptors MD-2, CD14, and LPB to facilitate high affinity binding to TLR-4 and subsequent host cell activation), bacterial lipoproteins (LPs), lipoteichoic acid, flagellin, membrane vesicles, and exopolysaccharides. Examples of PRRs expressed by host cells in the gut and resident intestinal immune cells that can mediate modulation of cytokine production via interaction with MAMPS include Toll-like receptors (TLRs), nucleotide-binding oligomerization domains (Nods), NOD like receptors and C-type lectins. The interaction of intestinal cell PRRs and microbial ligands trigger signaling pathways associated with the innate and adaptive immune systems that are required to maintain immune tolerance and intestinal health. In an aspect, MAMPS exist on the surface of intact non-viable cells incorporated into a composition.

The present disclosure therefore provides for the treating of a patient with a “dead” or non-viable fecal bacterial preparation manufactured by inactivating or removing the viability of a fecal microbiota or fecal bacterial preparation (e.g., by pasteurization and/or sonication) such that the non-viable fecal bacterial preparation retains therapeutic activity to treat conditions, disorders and diseases disclosed herein, for example via interactions between bacterial cellular surface molecules or membrane vesicles of the preparation and host signaling pathways associated with disease. The non-viability of the preparations disclosed herein provides a safer product compared to conventional live microbial therapeutics such as FMT-based compositions, as any pathogens that may have been present in a stool used as a source for the fecal bacterial preparation are inactivated during the manufacturing process and therefore not viable in the final pharmaceutical composition.

In an aspect, cellular substances are further enriched in a composition by subjecting a preparation containing ruptured cells (e.g., by sonication) to a process that purifies or concentrates membrane vesicles. For example, a preparation of sonicated cells extracted from a fecal microbiota can be subjected to ultracentrifugation and/or successive filtration steps ending with ultrafiltration to capture membrane vesicles.

Disclosed herein are methods of manufacturing a pharmaceutical composition comprising receiving a stool or portion thereof from a human donor, subjecting the fecal microbiota of the stool to treatments or sterilizing agents (e.g., heat, radiation, sonication) to generate a non-viable fecal bacterial preparation, and incorporating the fecal bacterial preparation into the composition. In certain aspects, prior to generating the non-viable fecal bacterial preparation, fecal bacteria extracted from a stool of a donor can be exposed to a germinant to induce germination of any bacterial endospores present in the stool. Non-limiting examples of germinants include a nutrient germinant (e.g., an amino acid such as glutamic acid, proline, leucine and combinations thereof, potassium bromide, glucose, and combinations thereof), pyridine-2,6-dicarboxylic acid (dipicolinic acid [DPA]) in a 1:1 chelate with divalent cations, such as Ca²⁺ (CaDPA), Dodecylamine, peptidoglycan fragments, and/or high pressure (e.g., 100 to 350 megaPascals or 500 to 1000 megaPascals). Such exposure to germinants ensures that any spores that may be present in a fecal bacterial preparation are converted to a vegetative state that is susceptible to the effect of treatment with sterilizing agents.

In aspects of the present disclosure, a pharmaceutical composition comprises a bacterial mixture comprising a non-viable fecal bacterial preparation supplemented or “spiked” with one or more bacterial isolates. By enriching or spiking a non-viable fecal bacterial preparation derived from a stool sample (e.g., a fecal microbiota) of a healthy donor with one or more non-pathogenic bacterial isolates, a composition can be produced in which the amount of a particular bacterial strain or strains (i.e., the spiked-in bacterial isolate(s)) can be accounted for and precisely controlled. Without being bound by theory, this is advantageous, for example, where the at least one bacterial isolate spiked into the fecal bacterial preparation is important for or involved in the treatment of a subject, but insufficient on its own to generate an enhanced or optimal treatment response in the subject. Probiotics are relied upon for a biological effect associated with the administration of a single bacterial isolate or a few bacterial isolates. Unlike probiotics, however, administration to a subject of one or more bacterial isolates together with a non-viable fecal bacterial preparation (i.e., derived from a healthy donor) provides the subject with the advantage of the administered bacterial isolate combined with multi-factorial benefits conferred by the inactive cells and cellular components of the non-viable fecal bacterial preparation. These non-viable cells and cellular components may combine to, for example, provide for the necessary context or interactions (e.g. via one or more MAMPS on a membrane vesicle or surface of a bacterial cell) to enable cells of the bacterial isolate to induce an optimal response in the subject, or may directly induce a response in the subject that combines and/or synergizes with a response induced by the bacterial isolate to treat the subject. Accordingly, in certain aspects, a pharmaceutical composition comprising a mixture of one or more bacterial isolates and a non-viable fecal bacterial preparation can be more effective in treating a subject than a composition comprising the bacterial isolate alone.

In an aspect, a pharmaceutical composition comprises a bacterial mixture comprising a fecal bacterial preparation, for example non-selected fecal bacteria and/or a substantially complete fecal microbiota of a stool or portion thereof (e.g., from a healthy human donor) treated with one or more sterilizing agents. In an aspect, a substantially complete fecal microbiota is a preparation of fecal bacteria that comprises bacteria from all or substantially all of the bacterial taxa represented among the bacteria in the stool from which the fecal microbiota was extracted. In an aspect, the relative abundance of bacteria from at least two of the taxa in the substantially complete fecal microbiota (comprising non-viable bacteria) is proportional to the relative abundance of those taxa in the stool from which the fecal microbiota was extracted. In an aspect, the bacterial mixture further comprises one or more bacterial isolates. In an aspect, the bacterial mixture does not comprise a bacterial isolate.

In one aspect, a fecal bacterial preparation to be subjected to a treatment to generate non-viable cells or ruptured cells or components thereof comprises a donor’s entire or substantially complete fecal microbiota from a stool or portion thereof. In one aspect, a fecal bacterial preparation comprises a non-selective fecal microbiota. In another aspect, a fecal bacterial preparation comprises an isolated or purified population or community of non-pathogenic fecal bacteria. In a further aspect, a fecal bacterial preparation comprises a non-selective and substantially complete fecal microbiota preparation from a single donor. In another aspect, a pharmaceutical composition used herein comprises a mixture of non-pathogenic, bacterial isolates and a non-viable, non-pathogenic, purified or extracted fecal bacterial preparation.

In an aspect, the manufacture of a preparation of a preparation of fecal bacteria from a stool (i.e., such preparation to be subjected to a further treatment for generating a non-viable fecal bacterial preparation) involves a treatment selected from the group consisting of ethanol treatment, detergent treatment, heat treatment, irradiation, and sonication. In another aspect, the manufacture of a preparation of fecal bacteria from a stool involves no treatment selected from the group consisting of ethanol treatment, detergent treatment, heat treatment, irradiation, and sonication. In one aspect, the manufacture of a preparation of fecal bacteria from a stool involves a separation step selected from the group consisting of density gradients, filtration (e.g., sieves, nylon mesh), and chromatography. In another aspect, the manufacture of a preparation of fecal bacteria from a stool involves no separation step selected from the group consisting of density gradients, filtration (e.g., sieves, nylon mesh), and chromatography. In another aspect, a preparation of fecal bacteria comprises an entire or substantially entire fecal microbiota from a stool or portion thereof of a subject. In another aspect, a pharmaceutical composition administered herein comprises a fecal microbiota substantially free of donor eukaryotic cells.

In an aspect, a method comprises administering a non-viable fecal bacterial preparation to a subject in need thereof, wherein bacterial components of the preparation treat or prevent a disorder in the subject by directly influencing non-immune system based host pathways. In another aspect, a method comprises administering a non-viable fecal bacterial preparation to a subject in need thereof, wherein bacterial components of the preparation treat or prevent a disorder in the subject by directly influencing immune system-based host pathways, as further described herein.

Disclosed herein are pharmaceutical compositions (e.g., vaccines or a composition that activates or suppresses the immune system) comprising one or more microbial (e.g., pathogen) antigens (e.g., in the form of an attenuated or inactive virus or a portion thereof, or a bacterial protein (e.g. endotoxin) or portion thereof) and a non-viable fecal bacterial preparation derived from a stool of a human donor. In an aspect, the pharmaceutical composition disclosed herein comprises one or more microbial (e.g., pathogen) antigens in combination with one or more adjuvants derived from a stool of a human donor, e.g., in the form of a non-viable fecal bacterial preparation. In one aspect, an adjuvant is or comprises cellular or non-cellular material, including intact bacterial cells, lysed or ruptured bacterial cells and components thereof, bacterial cellular vesicles and bacterial-produced or derived metabolites, macromolecules or compounds. Herein the term “microbe” or “microbial” encompasses viruses and any microscopic cellular organism (e.g., unicellular organism), including prokaryotes such as bacteria and unicellular eukaryotes, for example protists and protozoa (e.g., Plasmodium). In certain aspects, a microbe is a pathogen. Herein the term “pathogen” refers to any microbe (e.g., bacteria, protist, protozoa, virus) capable of infecting a host (e.g., a human) and causing a disease. Herein the term “antigen” refers to any molecular structure capable of being recognized by a T-cell receptor and/or antibody. Examples of antigens include peptides or portions thereof, polysaccharides or portions thereof, or a combination thereof (e.g., as a glycoprotein). In some cases an antigen can include lipids and/or nucleic acids when combined with a protein or polysaccharide. Herein the term “microbial antigen” refers to an antigen that originates from a microbe.

In an aspect, a pharmaceutical composition (e.g., vaccine or a composition that activates or suppresses the immune system) disclosed herein comprises one or more antigens from a virus or a component thereof. Herein the terms “virus”, “virion”, and “virus particle” are used interchangeably and broadly refer to a submicroscopic infectious agent that replicates inside of a living cell of an organism. A virus can be an RNA virus or a DNA virus. Examples of classes of an RNA virus include a dsRNA virus (e.g., Reovirus), (+) ssRNA virus (e.g., Coronavirus, Picornavirus, Togavirus), (-) ssRNA virus (e.g., Orthomyxovirus, Rhabdovirus), and ssRNA-RT viruses (e.g., retrovirus). Examples of classes of DNA viruses include a dsDNA virus (e.g., Adenovirus, Herpesvirus Poxvirus), ssDNA virus (e.g., Parvovirus), and dsDNA-RT virus (e.g. Hepadnavirus). In an aspect, a virus can comprise an envelope. In another aspect, a virus does not comprise an envelope.

In an aspect, a pharmaceutical composition (e.g., vaccine) disclosed herein comprises an antigen from a (+) ssRNA virus or a component thereof. For example, the (+) ssRNA virus can be selected from the group consisting of a Hepacivirus C, West Nile virus, Dengue virus, and a coronavirus. Non-limiting examples of a coronavirus include a severe acute respiratory syndrome coronavirus (SARS-CoV), a severe acute respiratory syndrome coronavirus 2 (SARS-CoV-2) and a Middle East respiratory syndrome-related coronavirus (MERS-CoV). In an aspect, a pharmaceutical composition disclosed herein comprises an antigen (e.g., peptide-based antigen) from SARS-CoV-2. Herein “SARS-CoV-2” refers to an enveloped positive sense single-stranded RNA virus that is a strain of the species Severe acute respiratory syndrome-related coronavirus (SARS-CoV) and a member of the genus Betacoronavirus and subgenus Sarbecoronavirus. SARS-CoV viruses (including SARS and SARS-CoV-2) are thought to enter host cells by binding to the angiotensin converting enzyme 2 (ACE2) receptor.

In an aspect, a pharmaceutical composition (e.g., vaccine) disclosed herein comprises one or more antigens from a virus (e.g., active virus, inactive virus, or attenuated virus) or portion thereof selected from the group consisting of Dengue virus, Hepatitis A virus, a Hepadnavirus, Hepatitis B virus, Hepatitis C virus, Hepatitis D virus (HDV), Hepatitis E virus, Human papillomavirus, an Orthomyxovirus, Influenza virus (e.g., Influenza virus A, Influenza virus B, Influenza virus C, or Influenza virus D) , Japanese encephalitis virus, Polio virus, Rotavirus, Rubella virus, Variola virus, Adenovirus, Coxsackie B virus, Enterovirus 71, Human immunodeficiency virus, Human T-cell lymphotropic virus (HTLV-I), Norovirus, Respiratory syncytial virus, Poxvirus, Vaccinia virus, Paravaccinia virus, Cowpox virus, Monkeypox virus, Bovine popular stomatitis virus, Orf virus, Smallpox virus, Herpesvirus, Herpes simplex virus (HSV-1 or HSV-2), Varicella zoster virus (VZV or HHV-3), Epstein-Barr virus (EBV or HHV-4), Cytomegalovirus (HCMV or HHV-5), Human herpesvirus 6A (HHV-6A), Human herpesvirus 6B (HHV-6V), Human herpesvirus 7 (HHV-7), Kaposi’s sarcoma-associated herpesvirus (KSHV or HHV-8), Tanapox virus, Yaba monkey tumor virus, Molluscum contagiosum virus, a Flavivirus, West Nile virus, Tick-bome encephalitis virus, Yellow fever virus, Zika virus, Palm Creek virus (PCV), Parramatta River virus (PaRV), an Alphavirus, the Barmah Forest virus, Chikungunya virus, Mayaro virus, O′nyong′nyong virus, Ross River virus, Semliki Forest virus, Sindbis virus, Una virus, Eastern Equine encephalitis virus, Tonate virus, Venezuelan equine encephalitis virus, Western equine encephalitis virus, an Asfaviridae, Togavirus, Paramyxovirus, Measles virus, Mumps virus, Rhabdovirus, Rabies virus, Bunyavirus, Crimean-Congo hemorrhagic fever orthonairovirus (CCHFV), Hantavirus, Puumala orthohantavirus (PUUV), a Filovirus, Ebola virus, Marburg virus, and a combination thereof.

A pharmaceutical composition (e.g., vaccine) can comprise one or more live or active viruses. In another aspect, a pharmaceutical composition (e.g., vaccine) can comprise one or more inactive viruses. In a further aspect, a pharmaceutical composition (e.g., vaccine) can comprise inactive viruses only, and lack active viruses (e.g., of a particular type of virus, such as a coronavirus). Herein the term “active” with respect to a virus refers to the capacity of the virus to replicate itself by binding and entering into (i.e., infecting) a cell. In contrast, herein an “inactive” virus refers to a “killed” virus that lacks the capacity to replicate by infecting a cell (e.g., due to treatment of the virus with ultraviolet light or heat). In a further aspect, a pharmaceutical composition (e.g., vaccine) can comprise one or more attenuated viruses or a portion thereof. Herein an “attenuated” virus refers to a virus that has been modified from its “wildtype” form (e.g., by the acquisition of one or more genetic mutations, for example a deletion) to reduce its virulence in a host. While an attenuated virus may replicate in a host, it typically does so at a lower rate than its wildtype counterpart.

In an aspect, a pharmaceutical composition (e.g., vaccine) described herein comprises a component or portion of a virus, either in addition to or in the absence of an intact virus (e.g., an inactive, active or attenuated intact virus). Typically such a component of a virus comprises a peptide or portion thereof. Herein the terms “peptide”, “polypeptide” and “protein” are used interchangeably and refer to a natural polymer of at least two amino acids linked together by peptide bonds. In an aspect, a viral peptide or portion thereof incorporated into a pharmaceutical composition can be at least 5, at least 10, at least 20, at least 50, at least 75, at least 100, at least 150, at least 200, at least 250, or at least 300 amino acids. A peptide herein includes a glycoprotein, for example, a glycoprotein of a viral envelope. In an aspect, a peptide or portion thereof is denatured or partially denatured.

A pharmaceutical composition (e.g., a vaccine) described herein can comprise any viral peptide as an antigen, which when administered to a subject results in the production of antibodies in the subject against such antigen. For example, a viral antigen incorporated into a composition can comprise a portion of the virus that functions as a receptor in a host to adhere or adsorb the virus to a host cell. In some cases, such a receptor can be present on a viral capsid or a viral envelope. An example of such a receptor that can be incorporated into a composition as a viral antigen is the S (spike) protein of SARS-CoV-2 (e.g., the S1 or RBD domain of the S peptide). In other aspects, a viral antigen incorporated into a composition (e.g., a vaccine) does not function as a viral receptor in the host.

In an aspect, a pharmaceutical composition (e.g., vaccine) comprises one or more peptides (or portion thereof) originating from a coronavirus such as SARS-CoV-2. Examples of such peptides include the S (spike) protein, E (envelope) protein, M (membrane) protein, and N (nucleocapsid) protein.

Any method, means or process known in the art can be used to inactivate a virus and/or separate an intact virus into its component parts (e.g., viral peptides or portions thereof) and/or denature a virus or viral peptide. In an aspect, a virus is inactivated by treating the virus with heat. In an aspect, such heat treatment comprises a pasteurization step or an autoclaving step. For example, a virus can be inactivated by pasteurizing the virus (e.g., present in a stool, filtered and homogenized stool or a fecal filtrate) at 60° C. for approximately 10 hours. In other examples, a virus can be inactivated by heating the virus to at least 40° C., at least 45° C., at least 50° C., at least 55° C., at least 60° C., at least 65° C., at least 70° C., at least 75° C., at least 80° C., at least 85° C., at last 90° C., at least 95° C., at least 100° C., at least 100° C., at least 110° C., at least 120° C. (e.g., in an autoclave), or greater than 120° C. In non-limiting examples, such heating can be applied for at least 1 minute, at least 5 minutes, at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 15 hours, at least 20 hours, or at least 24 hours. In certain aspects, the virus can be heated under pressure (e.g., in an autoclave). Heating can be by any known means including by hot water (e.g., water bath) or steam or use of a plate heat exchanger. Other methods of inactivating a virus that can be employed with the methods described herein include treatment of the virus (e.g., present in a stool or fecal filtrate) with pressure (e.g., Pascalization), pulsed electric field electroporation, nonthermal plasma, irradiation (e.g., with UV light, beta particles, gamma rays, or a combination thereof), solvent and/or detergent (e.g.,Triton X-100), or with low pH (e.g., less than or equal to pH 4). Inactivation steps (e.g., heating) can be performed on a virus present in any environmental context or state of purification. For example, examples of substrates that can be heated and/or pasteurized to inactivate virus include a solution or pellet of purified virus (e.g., obtained from a stock center such as ATCC), blood or stool (e.g., raw stool) containing the virus, or a fecal filtrate as described herein.

In an aspect, pasteurization provides a reliable method to inactivate a pathogen (e.g., SARS-CoV-2 or a different virus) to protect the safety of a subject administered a composition. For example, ten emerging viral threats were spiked into solution and measured at regular time points for viral titer, and all ten viruses were completely inactivated within six hours of pasteurization at 60° C. (Gröner, A. et al. (2018) ‘Effective inactivation of a wide range of viruses by pasteurization’, Transfusion. Blackwell Publishing Inc., 58(1), pp. 41-51. doi: 10.1111/trf.14390, all of which is hereby incorporated by reference herein).

In certain aspects, an antigen (e.g., viral peptide such as a viral glycoprotein) of a virus that is pathogenic to humans can be incorporated into a pharmaceutical composition (e.g., vaccine) by selecting a stool of a human donor on the basis of the presence of the virus in the stool, heating (e.g., pasteurizing) the stool comprising the pathogenic virus to inactivate the virus, homogenizing and filtering the heated stool to remove bacteria and at least some intact forms of the pathogenic virus, optionally lyophilizing and/or encapsulating the fecal filtrate, i.e., comprising a non-viable fecal bacterial preparation, and administering the resulting pharmaceutical composition to a human subject as a vaccine to vaccinate or immunize the subject against the pathogenic virus (i.e., to prevent an infection of the subject by the pathogenic virus and/or to prevent a disease or disorder resulting from an infection in the subject by the pathogenic virus). In addition to inactivating the virus, the heating and filtering steps can break apart or dismantle the intact viral particles into their component viral peptides, which can pass through the filter pores into the fecal filtrate thus providing for a final composition that is safe to administer to a subject (i.e., non-infectious) and effective to induce an immune response against the viral antigen. As described herein, the non-viral components of the filtrate/non-viable fecal bacterial preparation (e.g., bacterial metabolites) can function to enhance the efficacy of the vaccine by conferring adjuvant (immune-stimulating) activity in the gut of the subject following administration of the composition. In another aspect, a viral antigen may not exist in the stool of a donor, or in the filtered and homogenized stool of the donor, and instead a fecal filtrate can be supplemented with the viral antigen (e.g., a peptide produced from a recombinant source, intact viral particles that have been inactivated or attenuated, or component parts of a virus produced for example by heating a preparation of intact virus).

In an aspect, the pathogenic virus is a coronavirus and the vaccine is administered to prevent severe acute respiratory syndrome (SARS) or coronavirus disease 2019 (COVID-19). In an aspect, the pathogenic virus is SARS-CoV-2 and the vaccine is administered to prevent COVID-19. Herein “COVID-19” refers to an infectious disease caused by SARS-CoV-2, with primary symptoms including fever, coughing, and shortness of breath. Other symptoms may include fatigue, headache, nasal congestion, chills, nausea and/or vomiting, muscle pain, diarrhea, sore throat, loss or reduction in sense of smell (hyposmia or anosmia), abdominal pain, hemoptysis, expectorating, confusion, difficulty waking, persistent chest pain, reduced white blood cell count, and kidney failure.

Further disclosed herein are pharmaceutical compositions (e.g., vaccines) comprising an immunomodulator comprising an antigen (e.g., comprising a peptide and/or a polysaccharide) derived from a non-viral microbe (e.g., a unicellular pathogenic bacteria, protist or protozoa). For example, the antigen can comprise a toxin or portion thereof (e.g., bacterial toxin or portion thereof), which when produced and/or released by the bacteria in an infected host can contribute to the development of a disease or disorder. In another example, the antigen can comprise a toxoid derived from a toxin produced or released by a non-viral pathogen (e.g., bacteria). Herein the term “toxoid” refers to an inactivated or attenuated form of a toxin, the toxicity of which has been suppressed by for example chemical or heat treatment. In an aspect, a composition disclosed herein comprises a toxoid comprising inactivated or attenuated tetanospasmin (i.e., tetanus toxin derived from Clostridium tetani). In another other aspect, a composition disclosed herein comprises a toxoid comprising inactivated or attenuated botulin (i.e., botulinum toxin derived from Clostridium botulinum). In another aspect, a composition disclosed herein comprises a toxoid comprising inactivated or attenuated diphtheria toxin (i.e., derived from Corynebacterium diphtheriae). In another aspect, a pharmaceutical composition (e.g., vaccine) does not comprise an antigen or peptide derived from a toxin or a toxoid.

A pharmaceutical composition (e.g., vaccine) disclosed herein can comprise an immunomodulator comprising a bacterial antigen (e.g., from a bacterial pathogen), which when administered to a subject results in the production of antibodies in the subject against such bacterial antigen. In an aspect, a bacterial antigen incorporated into a composition can originate from any known species of bacteria (e.g., any known bacterial pathogen), including but not limited to Mycobacterium sp., Mycobacterium tuberculosis, Mycobacterium bovis (e.g., Mycobacterium bovis BCG), Corynebacterium sp., Corynebacterium diphtheriae, Clostridium sp., Clostridium tetani, Bordetella sp., Bordetella pertussis, Haemophilus sp., Haemophilus influenzae (e.g., type b), Vibrio sp., Vibrio cholerae, Salmonella sp., Salmonella enterica (e.g., Salmonella enterica subsp. enterica), Salmonella typhimurium, Salmonella typhi, Streptococcus sp., Streptococcus pneumoniae, Fusobacterium sp., Fusobacterium nucleatum, Listeria sp., Listeria monocytogenes, Listeria ivanovii, Listeria grayi, Yersinia sp., Yersinia pestis, enterocolitica, Yersinia pseudotuberculosis, Shigella sp., Shigella dysenteriae, Shigella flexneri, Shigella boydii, Shigella sonnei, Neisseria sp., Neisseria meningitidis, Bacillus sp., Bacillus anthracis, Francisella tularensis, Rickettsia prowazekii, Coxiella burnetiid, and a combination thereof.

A pharmaceutical composition (e.g., vaccine) can further comprise an immunomodulator comprising an antigen derived from a eukaryotic unicellular organism (e.g., a unicellular pathogen). In an example, such antigen comprises a peptide, polysaccharide or portion thereof from the genus Plasmodium, for example from the species P. falciparum, P. vivax, P. ovale, P. malariae, P. knowlesi, or a combination thereof. In an aspect, a peptide incorporated into a pharmaceutical composition comprises the circumsporozoite protein (CSP) from Plasmodium sp.

A pharmaceutical composition can comprise an immunomodulator comprising one or more antigens from a cellular microbe (e.g., bacteria) that is pathogenic to humans. In an aspect, a bacterial antigen from pathogenic bacteria is obtained by selecting a stool of a human donor on the basis of the presence of the pathogenic bacteria in the stool, filtering the stool to remove the pathogenic bacteria from the resulting fecal filtrate, optionally lyophilizing and/or encapsulating the fecal filtrate, and administering the resulting pharmaceutical composition to a human subject to vaccinate or immunize the subject against the pathogenic bacteria (i.e., prevent an infection by the bacterium and/or prevent a disease or disorder that is caused by the bacterium). In addition to removing the pathogenic bacteria from the resulting fecal filtrate, the filtering step can further break apart or dismantle the pathogenic bacteria into component parts (e.g., cell wall, metabolites, macromolecules such as peptides and polysaccharides) that can pass through the pores of the filter and thereby be incorporated as the antigen(s) in the final pharmaceutical composition. In an aspect, the filtering step can be accompanied or replaced by an inactivation step that either kills the bacteria or attenuates their activity. For example, the stool of a donor comprising pathogenic bacteria (i.e. selected on the basis of the presence of pathogenic bacteria) or the fecal filtrate resulting from filtration of the stool of a donor comprising pathogenic bacteria can be subjected to an inactivation step comprising for example treating with heat (e.g., pasteurization, for example at 60° C. for about 10 hours), pressure (e.g., Pascalization), pulsed electric field electroporation, nonthermal plasma, irradiation (e.g., with UV light, beta particles, gamma rays, or a combination thereof), solvent and/or detergent (e.g., Triton X-100), or with low pH (e.g., less than or equal to pH 4). In another aspect, an antigen of a pathogenic cellular microbe may not be incorporated into a composition via processing of a stool of a donor comprising the bacteria, but instead filtered and homogenized stool can be supplemented with the bacteria (e.g., heat-killed bacteria) or one or more component parts of the bacteria (e.g., a recombinant peptide) to supply the antigen in the final vaccine composition.

In an aspect, a pharmaceutical composition comprises an immunomodulator comprising a microbial (e.g., pathogen) antigen (e.g., active, inactive or attenuated virus or bacteria or a viral or bacterial peptide or portion thereof) that is capable of inducing an immune response in a subject administered the composition. Such an immune response can be induced for example by an immunogenic epitope of the antigen. For example, without wishing to be bound by theory, a pharmaceutical composition (e.g., an orally delivered enteric encapsulated pharmaceutical composition comprising a fecal filtrate or filtered and homogenized stool) can deliver a microbial antigen (e.g. peptide) to the intestine (e.g., small intestine or large intestine) of the subject, where one or more immunogenic epitopes of the antigen can adhere to the surface of an intestinal cell (e.g., intestinal epithelial cell), thereby inducing an immune response in the subject. In a non-limiting example, the epitope(s) can be recognized by the angiotensin-converting enzyme 2 (ACE2) receptor of an intestinal cell. In an aspect, at least part of the epitope is found on or is a portion of the “S” or “spike” protein of a coronavirus that is recognized by the ACE2 receptor of intestinal epithelial cells. In an aspect, the coronavirus is SARS-CoV-2 and the spike protein was administered to the subject to prevent infection by SARS-CoV-2 and/or to prevent COVID-19.

In an aspect, the immune response produced or induced by administration of a pharmaceutical composition comprising a pathogen antigen to a subject comprises at least one of a humoral immune response and a cell-mediated immune response. In an aspect, the humoral immune response comprises production of one or more immunoglobulins or antibodies (e.g., by a B cell) that recognize and are capable of binding to at least one epitope of the active pathogen present in a subject administered the pharmaceutical composition. Accordingly, administration to a subject of a pharmaceutical composition described herein comprising a pathogen antigen constitutes a vaccination of the subject by inducing an immune response that includes generation of immunoglobulins capable of recognizing and neutralizing the active infectious pathogen. Such immunoglobulins can include one or more of IgG, IgA, IgD, IgM or IgE. In an aspect, an immunoglobulin comprises a neutralizing antibody. In a further aspect, one or more antibodies generated in response to exposure of the subject to a microbial antigen is a neutralizing antibody. Herein a “neutralizing antibody” is an antibody capable of defending a cell of a host from an antigen of an infectious microbe by inhibiting or neutralizing the antigen’s biological effect. In one example, a neutralizing antibody generated by a subject in response to administration of a composition described herein is capable of binding to an epitope of the spike protein of a coronavirus, e.g., SARS-CoV-2.

An immune response generated by administration to a subject of a composition described herein can include a cell-mediated immune response. For example, the cell-mediated immune response can comprise activation of one or more of T helper cells (CD4⁺ cells) and cytotoxic T lymphocytes. A benefit of inducing a cell-mediated response is that smaller epitopes, including non-conformational epitopes, are often sufficient for such induction. As a result, epitopes sufficient for induction of a T-cell response may be preserved following inactivation of the pathogen (e.g., following heat inactivation by for example pasteurization or autoclaving). A further benefit of a composition comprising epitopes that induce cell-mediated immunity is that such epitopes can be found on “structural” intra-microbial (e.g. intraviral) portions of a protein, and as such not on the surface of the virus, in contrast to for example surface glycoproteins. In an aspect, an epitope that contributes to inducing a cell-mediated response in a subject administered a composition is a portion of a viral peptide that in the native virus exists as an intraviral protein, i.e. a segment of a protein that is not host-interacting and/or accessible to an antibody in contact with the intact virus. Such an epitope can be in a greater state of evolutionary conservation than host-interacting epitopes, giving it a slower rate of evolution and more consistent immunogenic capability across individuals and over time, i.e. so that a vaccine comprising the epitope remains effective across individuals for a longer period of time compared to a vaccine comprising host-interacting epitopes. In an aspect, a composition comprises an intraviral epitope capable of inducing a cell-mediated immune response in a subject. In an aspect, the intraviral epitope is from a coronavirus, for example, SARS-CoV-2.

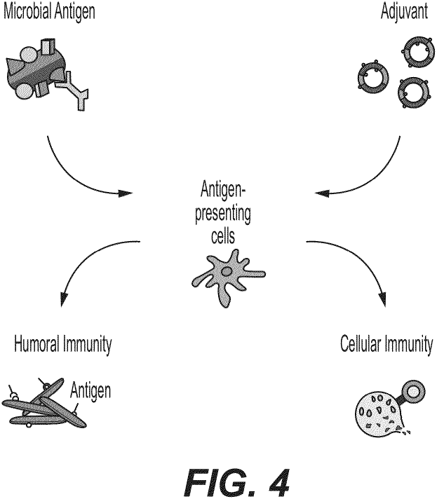

In an aspect, a pharmaceutical composition (e.g., vaccine) described herein induces an immune response comprising both humoral immunity and cellular immunity. Without wishing to be bound by theory, FIG. 4 shows an example mechanism of action for a vaccine comprising a microbial antigen comprising SARS-CoV-2 peptides and an adjuvant. Three proteins expressed in SARS-CoV-2 (S, N and M proteins) are predicted to include epitopes capable of electing a B-cell and T-cell mediated immune response (Griffoni et al. (2020) Cell Host Microbe 27: 671-680, hereby incorporated herein by reference in its entirety). For the adjuvant, LPS, flagellin, lipoproteins peptidoglycan fragments and other bacterial debris delivered to the small bowel will serve as potent adjuvants, helping overcome mucosal tolerance. Bacterial lysates have previously demonstrated significant TLR4 mediated adjuvancy for protective antibody responses against respiratory viruses in mice. In addition, as described herein, antigens delivered in the composition can be partially bound to secretory IgA which will increase antigen sampling relative to native epitopes. The complex adjuvant activity of the composition can correspondingly allow induction of a significant, protective mucosal and systemic immune response. Exposure to SARS-CoV-2 epitopes in the small bowel, coupled with the adjuvant effect of bacterial components is expected to lead to production of anti-SARS-CoV-2 IgA and IgG antibodies, providing lasting humoral immunity. In addition, the nature of the adjuvant activity will promote MHC cross presentation of T-cell epitopes and induce cellular immunity which may increase vaccine efficacy.

In an aspect, a pharmaceutical composition disclosed herein is a vaccine. Herein a “vaccine” refers broadly to a biological preparation that induces acquired immunity to a particular infectious disease in a subject administered the preparation. Non-limiting examples of vaccines include live vaccines, live, attenuated vaccines, inactivated vaccines, toxoid vaccines, subunit vaccines and conjugate vaccines. Herein a “conjugate vaccine” refers to a vaccine that comprises an immunogenically weak antigen (e.g., a polysaccharide from a surface coating of a pathogenic bacteria) from a human pathogen linked (e.g., covalently linked) to an immunogenically strong antigen (e.g., a peptide of the pathogen such as a toxin, portion thereof, or a toxoid generated from the toxin) to promote production of antibodies recognizing the immunogenically weak antigen. Herein a “subunit vaccine” is a vaccine that comprises only a portion or component of a pathogen, which has been generated de novo or purified or separated from the remaining non-included portions or components. An example is a peptide of a microbe that has been generated via recombinant techniques.

In an aspect, a non-viable fecal bacterial preparation comprises one or more substances, such as bacterial cells (e.g., bacterial cells), macromolecules, compounds, or metabolites, derived from a fecal microbiota of the stool donor that exhibit adjuvant activity when administered in a pharmaceutical composition in combination with microbial antigen described herein. Herein the term “adjuvant” refers to an agent that when administered to a subject, for example in combination with an immunogenic antigen (e.g., antigen of a viral or bacterial pathogen), boosts or potentiates the subject’s resulting immune response to the antigen, for example by increasing the quantity of antibodies, their rate of production, or the duration of their production. Non-limiting examples of adjuvants derived from a stool or fecal microbiota of a donor that can be present in a non-viable fecal bacterial preparation include a peptide, a lipopolysaccharide, flagellin, a peptidoglycan, a lipoteichoic acid, a lipoprotein, or a membrane vesicle. In an aspect, a concentration or availability of at least one of such adjuvants is increased in a fecal filtrate or a non-viable fecal bacterial preparation by applying a heating, homogenization and/or filtration step described herein. For example, raw stool from a donor can be pasteurized (e.g., at or about 60° C. for approximately 10 hours) and then homogenized and filtered to separate one or more adjuvant-potent bacterial-derived compounds from its origin bacterial cell, thereby making the compound available in the non-viable fecal bacterial preparation as an adjuvant when the pharmaceutical composition is administered to a subject. In other examples, a heating or pasteurization step is applied following filtration. Without wishing to be bound by theory, an adjuvant described herein can potentiate an immune response by for example extending the length of time a microbial antigen is available in an intestine to induce an immune response, facilitating the presentation of the antigen to a host cell for induction of the immune response, facilitating the binding of the antigen to a host cell surface or receptor, enhancing the rate of activation or quantity of activated host immune cells such as macrophages and lymphocytes, and/or supporting the production of cytokines by a cell of a host/subject administered the composition.

The present disclosure also contemplates that a pharmaceutical composition or non-viable fecal bacterial preparation can be supplemented with one or more adjuvants to increase an immune response induced by a microbial antigen administered in a pharmaceutical composition. Non-limiting examples of a adjuvants that can be incorporated into a pharmaceutical composition described herein include inorganic compounds such as alum, aluminum hydroxide, aluminum phosphate, and calcium phosphate hydroxide, mineral oil, a cytokine such as IL-1, IL-2 or IL-12 a combination adjuvant such as Freund’s complete adjuvant, Freund’s incomplete adjuvant or AS01, or a food-based oil such as Adjuvant 65.

In an aspect, the present disclosure contemplates that a fecal filtrate or pharmaceutical composition can be supplemented with a general non-donor-derived adjuvant or vaccine adjuvant. In one aspect the “adjuvant” or “vaccine adjuvant” are molecules or compounds that have intrinsic immunomodulatory properties and, when administered in conjunction with an antigen, effectively potentiate the host antigen-specific immune responses compared to responses raised when antigen is given alone. See Pasquale, A., et al., Vaccine Adjuvants: from 1920 to 2015 and Beyond. Vaccines 3(2): 230-343 (2015).

In an aspect, a pharmaceutical composition comprises a stool-based adjuvant (e.g., in the form of a non-viable fecal bacterial preparation) but does not comprise a pathogen or antigen thereof. In an aspect, a non-viable fecal bacterial preparation is derived from a disease-screened donor and is free of pathogenic bacteria.

In aspects, the compositions described herein can be administered to a patient to boost the immune system by providing non-infectious bacterial or microbial components that confer adjuvant activity.

In certain aspects, such adjuvant activity and immune system boosting is provided in the context of another treatment that acts in a subject by inducing an immune response, i.e., an immunotherapy. Herein an “immunotherapy” refers to the administration of an immunomodulator to a subject to induce an immune response in the subject. Such an immune response can be induced for example by administering to a subject an antibody or derivative thereof (e.g., for an anti-cancer immunotherapy) or an immunogenic epitope of an antigen (e.g., for an allergen immunotherapy). In an aspect, the immune response produced or induced by administration of an immunotherapy comprises at least one of a humoral immune response and a cell-mediated immune response. In an aspect, the humoral immune response comprises production of one or more immunoglobulins or antibodies (e.g., by a B cell) that recognize and are capable of binding to at least one epitope of an antigen (e.g., allergen). In an aspect, the immune response comprises the recognition of an epitope presented by a tumor by an antibody administered to the subject via the immunotherapy. Herein an “immunomodulator” refers to any composition or formulation or preparation or compound that is capable of inducing an immune response. In certain aspects, an immunomodulator comprises a pathogen antigen administered in a vaccine to promote an immune response leading to vaccination of the subject against the pathogen. For example, a fecal bacterial preparation can comprise an antigen immunomodulator by manufacturing the preparation from stool of a donor who is infected or has recently been infected with the pathogen. In other aspects, an immunomodulator comprises an immunotherapy such as an anti-cancer immunotherapy or an allergen immunotherapy.

An immune response generated by administration to a subject of an immunomodulator can include a cell-mediated immune response. For example, the cell-mediated immune response can comprise activation of one or more of T helper cells (CD4⁺ cells) and cytotoxic T lymphocytes. In an aspect, an immunomodulator induces an immune response comprising both humoral immunity and cellular immunity.

For example, the compositions described herein can be administered to facilitate the efficacy of an immunotherapy selected from an anti-cancer immunotherapy and an anti-allergy therapy (allergen immunotherapy or allergy vaccine therapy). In aspects, a pharmaceutical composition can be administered to enhance the efficacy of an anti-cancer immunotherapy selected from the group consisting of dendritic cell-based pump-priming or vaccination, T-cell adoptive transfer and checkpoint inhibitor therapy. In aspects, a pharmaceutical composition can be administered to enhance the efficacy of an allergen immunotherapy that treats or prevents a condition selected from the group consisting of allergic rhinitis, asthma, seasonal pollinosis, perennial allergy, allergy to dust mites and a food allergy, for example an allergy to peanuts, milk, eggs, tree nuts, fish or shellfish. In certain aspects, an allergen immunotherapy treats or prevents an allergy selected from the group consisting of an allergy to peanuts, soybeans, cow’s milk, milk protein, tree nuts, shellfish, fish, wheat, eggs, sesame, and/or dairy; seasonal allergies; outdoor allergies including grass and trees; animal or pet allergies; insect allergies; and mold allergies

In an aspect, a non-viable fecal bacterial preparation can be administered to a subject to provide for adjuvant activity to facilitate treatment of a disorder in combination with another treatment or therapy, for example an anti-cancer therapy in treatment of a cancer. In aspects, the immunotherapy comprises an immuno-oncology therapy, e.g., comprising administering a checkpoint inhibitor that binds and/or recognizes a checkpoint molecule, e.g., PD-1, PD-L1, PD-L2, ICOS, ICOSL, and CTLA-4. In aspects, the immuno-oncology therapy comprises administration of a checkpoint inhibitor selected from the group consisting of KEYTRUDA™ (Pembrolizumab), OPDIVO™ (Nivolumab), or YERVOY™ (Ipilimumab). In aspects, the immune-oncology therapy comprises administration of a checkpoint inhibitor that targets a tumor-cell antigen and/or a cancer-cell antigen selected from the group consisting of 2B4, A2AR, B-7 family ligands (including, but are not limited to, B7-1, B7-2, B7-DC, B7-H1, B7-H2, B7-H3, B7-H4, B7-H5, B7-H6 and B7-H7), BTLA, CD115, CD160/By55, CD172a/SIRPα, CD200, CD223, CD244, CEACAM, CHK 1 and CHK2 kinases, CTLA-4, GAL9, HVEM, IDO, KIR, LAG3, PD-1, PD-L1, PD-L2, TIGIT, TIM-3, TMIGD2, and VISTA/VSIG8, as described in PCT/US2019/165285, hereby incorporated herein by reference in its entirety.

In an aspect, an allergen immunotherapy administered to a subject (e.g., for treatment of asthma or a food allergy) comprises an oral immunotherapy comprising administering to the subject an allergen (e.g., a peanut allergen) for the purpose of increasing the threshold amount or concentration of the allergen required to stimulate an allergic reaction.

Administration of the compositions described herein can increase an innate immune system response relative to a treatment with an immunotherapy alone. The methods can also increase an adaptive immune system response, such as one or both of humoral immunity and cell-mediated immunity, relative to a treatment with the immunotherapy alone. Further, the methods can increase the number or activity of one or more of T cells, B cells, dendritic cells, macrophages, neutrophils, NK cells relative to a treatment with the immunotherapy alone. Administration of the compositions described herein may shift the ratio of immune cells in favor of cells that can kill and/or suppress a tumor or an allergen (e.g., T cells, cytotoxic T lymphocytes, T helper cells, natural killer (NK) cells, natural killer T (NKT) cells, anti-tumor macrophages (e.g., M1 macrophages), B cells, dendritic cells, or subsets thereof)) and in opposition to cells that protect tumors or facilitate allergies (e.g., myeloid-derived suppressor cells (MDSCs), regulatory T cells (Tregs), tumor associated neutrophils (TANs), M2 macrophages, tumor associated macrophages (TAMs), or subsets thereof) relative to treatments with the immunotherapy alone; for example, the methods increase the ratio of effector T cells to regulatory T cells. The methods may increase secretion of pro-inflammatory cytokines (e.g., interleukin-1 (IL-1), IL-12, and IL-18, tumor necrosis factor (TNF), interferon gamma (IFN-gamma), and granulocyte-macrophage colony stimulating factor) and/or decrease secretion of anti-inflammatory cytokines (e.g., interleukin (IL)-1 receptor antagonist, IL-4, IL-6, IL-10, IL-11, IL-13, interferon alpha (IFN-alpha, and transforming growth factor-beta (TGF-beta)) relative to treatments with the immunotherapy alone.

In aspects, administration of a composition described herein for increasing the efficacy of an immunotherapy can be carried out concurrently with the immunotherapy. For example, a pharmaceutical composition comprising a bacterial mixture or preparation as described herein can be administered to a patient while the patient is being treated with an immunotherapy. In other aspects, a composition for enhancing the efficacy of an immunotherapy can be administered after the immunotherapy. In other examples, a composition for enhancing the efficacy of an immunotherapy can be administered prior to the immunotherapy.

Disclosed herein is a method of treating a cancer in a subj ect in need thereof, the method comprising administering to the subject a pharmaceutical composition comprising a fecal bacterial preparation and an immunotherapy (e.g., a checkpoint inhibitor), wherein the fecal bacterial preparation does not contain viable bacterial cells. In another aspect, the fecal bacterial preparation administered to the subject does comprise viable bacterial cells, e.g., in the form of a bacterial mixture comprising one or more bacterial isolates and/or a preparation of fecal bacteria. In an aspect, the cancer is lung cancer (e.g., small-cell lung cancer, non-small cell lung cancer, adenocarcinoma of the lung, and squamous carcinoma of the lung). In an aspect, the cancer is selected from the group consisting of basal cell carcinoma, biliary tract cancer; bladder cancer; bone cancer; brain and central nervous system cancer; breast cancer (including triple-negative breast cancer); cancer of the peritoneum; cervical cancer; choriocarcinoma; colon and rectum cancer; connective tissue cancer; cancer of the digestive system; endometrial cancer; esophageal cancer; eye cancer; cancer of the head and neck (including carcinoma of head and neck); gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; urothelial cancer; uterine or endometrial cancer; cancer of the urinary system; vulval cancer; lymphoma including Hodgkin’s and non-Hodgkin’s lymphoma, B-cell lymphoma (including low grade/follicular non-Hodgkin’s lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom’s Macroglobulinemia; chronic lymphocytic leukemia (CLL); acute lymphoblastic leukemia (ALL); Hairy cell leukemia; chronic myeloblastic leukemia; other carcinomas and sarcomas; and post-transplant lymphoproliferative disorder (PTLD), abnormal vascular proliferation associated with phakomatoses, edema (such as that associated with brain tumors), and Meigs’ syndrome. In aspects, the cancer is bladder cancer, carcinoma of head and neck, colon and rectum cancer, kidney or renal cancer, melanoma, non-small cell lung cancer, triple-negative breast cancer, or urothelial cancer.

In aspects, the cancer is classified as PDL-1+ and/or CTLA4+.

Disclosed herein is a method of treating a condition or disorder that is activated or stimulated by exposure to an allergen in a subject in need thereof, the method comprising administering to the subject a pharmaceutical composition comprising a fecal bacterial preparation and an allergen immunotherapy, wherein the fecal bacterial preparation does not contain viable bacterial cells. In another aspect, the fecal bacterial preparation administered to the subject to treat an allergy does comprise viable bacterial cells, e.g., in the form of a bacterial mixture comprising one or more bacterial isolates and/or a preparation of fecal bacteria. In an aspect, the condition or disorder is selected from the group consisting of allergic rhinitis, asthma, seasonal pollinosis, perennial allergy, allergy to dust mites and a food allergy, for example an allergy to peanuts, milk, eggs, tree nuts, fish or shellfish.

In another aspect, a fecal bacterial preparation is produced from a donor’s stool that is “ready-made” with an antigen of a pathogen (e.g., a viral peptide or portion thereof). That is, a fecal bacterial preparation, for example in the form of filtered and homogenized stool and/or lacking or substantially lacking bacterial cells of the stool, can comprise the antigen of the pathogen that is administered to the subject in the pharmaceutical composition to induce an immune response, due to the presence of the pathogen in the stool used to produce the fecal bacterial preparation, along with the inherent adjuvant activity provided from a fecal bacterial preparation. In such cases, the stool donor may be infected with the pathogen (with or without symptoms), for example resulting in shedding of viral particles (if the pathogen is a virus) into the stool or incorporation of the pathogen into a fecal microbiota (if the pathogen is bacteria) of the donor. Collection of the stool from the donor followed by inactivation of the pathogen (e.g., by pasteurization) and filtration of the stool can produce a fecal filtrate-based vaccine that comprises both antigen and adjuvant for induction of an immune response against the pathogen in a subject administered the composition (i.e., as a pharmaceutical composition described herein). The platform described herein can thus leverage pathogen epitopes existing in stool of a donor as a source of both antigen and adjuvant for a vaccine.

In an aspect, a titer of virus in a stool of a donor is at least 10³ virions per gram of stool, at least 10⁴ virions per gram of stool, at least 10⁵ virions per gram of stool, at least 10⁶ virions per gram of stool, at least 10⁷ virions per gram of stool, at least 10⁸ virions per gram of stool, at least 10⁹ virions per gram of stool, or at least 10¹⁰ virions per gram of stool.

In other aspects, once prepared from the stool of a donor, the fecal filtrate can be supplemented or “spiked” with one or more microbial antigens (e.g., an inactivated virus or bacteria, immunogenic peptide or portion thereof obtained from a pure virus or bacterial stock).

In an aspect, a donor of stool that is used to produce a composition described herein is or has been recently infected with the pathogen (e.g., virus) that is the desired target of a vaccine disclosed herein. Such a donor can exhibit symptoms associated with such infection, or in other cases show no obvious symptoms. In an aspect, a donor or a stool collected from a donor can be screened for the presence or titer of a virus or bacterial pathogen, and the stool of the donor can be selected for processing only when the donor or the stool tests positive for the pathogen or when the titer of the pathogen exceeds a threshold amount. In an aspect, a donor is selected based on a predetermined level of an anti-SARS-CoV-2 Ig molecule (e.g., IgG, IgM, IgA). Thus, contrary to current practices the present disclosure provides for the selection of donor stool for downstream processing to produce a pharmaceutical composition comprising components of the stool (e.g., as filtered and homogenized stool or a fecal filtrate lacking or substantially lacking bacterial cells of the stool) on the basis of the presence of a known pathogen in the stool. In effect, the donor is employed as a bioreactor to co-generate pathogen antigen and adjuvant that is then administered (subject to inactivation and adjuvant-releasing steps such as pasteurization) to a subject as a vaccine in a pharmaceutical composition described herein.

In a case where a composition (e.g. vaccine) comprises antigen derived from a virus present in a stool of a donor, a benefit of harvesting viruses directly from stool is that the antigen is likely to be pre-labeled with IgA. Such labeling can advantageously facilitate antigen presentation and epitope stability during processing in the host, as compared to simply providing unlabeled viral protein in a vaccine. Without wishing to be bound by theory, one mechanism for this is that gut intestinal cells (e.g., in Peyer’s patches of the intestine) may more effectively take-up viral particles labeled with IgA, which can increase antigen sampling and antigen presentation, leading to increased antibody production by a subject. In addition, such IgA-labeling of antigens in stool can have a stabilizing effect by protecting and preserving epitopes during pathogen inactivation steps, for example heating.

Further disclosed herein are pharmaceutical compositions (e.g., vaccines) comprising (i) a microbial antigen bound to an antibody, and (ii) an adjuvant. In an aspect, the microbial antigen can be obtained pre-labeled with the antibody (e.g., IgA) directly from a stool of a donor. Alternatively, or in combination, the antigen can be obtained from a non-stool source, for example from a stock of an intact purified pathogen or synthesized using recombinant techniques, following which the antigen can be used to raise antibodies (monoclonal or polyclonal) specific to the antigen using known techniques. Likewise, in an aspect the adjuvant can be derived directly from a stool of a donor using methods described herein. Alternatively, or in combination, the adjuvant can comprise cell extracts of cultured bacteria. For example, the bacteria can comprise Salmonella or some other flagellated species of Gammaproteobacteria, the cell extracts of which deliver strong adjuvant activity. In another example, cell extracts of Mycobacterium bovis are used as an adjuvant.

The approach of using stool of a donor as a source of a microbial antigen for incorporation into a composition described herein further advantageously addresses the problem of escape mutations that are associated with traditional vaccination efforts in general and against coronaviruses in particular. The approach described herein elegantly solves this problem by continuously sampling antigens circulating in a donor population, allowing the antigens in the administered vaccine to mirror the antigens of the strain of the pathogen that are currently in the population. This is compared to conventional vaccines which employ an antigen that is fixed or static in the population at a moment in time, which permits the pathogen targeted by the vaccine to evolve away from the reach of the vaccine. In another aspect, a pharmaceutical composition can be tailored to the geographic and temporally relevant pathogen epitopes by harvesting antigens from donors sourced from local outbreaks, protecting against the most relevant variant impacting a given population.

In an aspect, disclosed herein is a method for manufacturing a vaccine against a viral pathogen (e.g., a virus that infects humans, such as SARS-CoV-2). The method comprises selecting a donor who is infected (or has been recently infected) with the virus, screening the stool of the donor for the presence of the virus in the stool, and processing the stool to produce fecal material (e.g., a fecal filtrate) that comprises an antigen of the virus (e.g., as a viral peptide or portion thereof) based on the detection of the virus in the stool. Such fecal material (e.g., fecal filtrate) can be administered to a subject to vaccinate or immunize the subject against the viral pathogen, prevent infection of the subject by the virus (e.g., SARS-CoV-2), and/or prevent a disease or disorder caused by the virus (e.g. COVID-19). Optionally, the fecal filtrate can be lyophilized and/or encapsulated. It will be understood that the above method is particularly suited to the manufacture of vaccines targeting a virus that is shed in the intestine of a subject infected with the virus (e.g., like SARS-CoV-2), such that the stool collected from the subject contains viral particles comprising one or more antigens. In an aspect, the collected stool or fecal filtrate is subject to a heating (e.g. pasteurization) step to inactivate the virus prior to incorporating the fecal filtrate into a pharmaceutical composition.

Disclosed herein is a method of manufacturing a pharmaceutical composition comprising selecting a stool of a human donor, wherein such selection is based on the existence of a pathogen in the stool, treating the stool (e.g., with heat) to inactivate the pathogen, and extracting fecal material from the stool (e.g., by homogenizing and filtering the stool) for incorporation into a pharmaceutical composition. In an aspect, the human donor is currently infected with the pathogen. For example, the donor can exhibit symptoms of an infection by the pathogen at the time the stool comprising the pathogen is collected from the donor. In another aspect, the human donor may not exhibit symptoms of the infection at the time of stool collection, but may have recently exhibited one or more symptoms of such an infection. For example, the donor may have exhibited symptoms of an infection by the pathogen 6 hours, 12 hours, 18 hours, 24 hours, 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks or 8 weeks prior to the collection of the stool comprising the pathogen from the donor. In an aspect, the pathogen is SARS-CoV-2 and the symptoms exhibited by the donor include a cough, fever and/or shortness of breath.

Selection of a donor and/or a stool comprising a pathogen, or determination of a titer or abundance or quantity of a pathogen in a stool, can be based on detection methods such as PCR or immunoassays. For example, a stool of a donor infected or recently infected with a pathogen can be tested using an immunoassay capable of specifically detecting an antigen unique to the pathogen, or with PCR (or RT-PCR in the case of an RNA virus) using primers designed to specifically target a nucleic acid sequence of the pathogen.

It will be understood that selection of a stool comprising a pathogen for processing to extract fecal material to be incorporated into a pharmaceutical composition described herein advantageously provides for the manufacture of a vaccine comprising an antigen of the pathogen in combination with one or more adjuvants endogenous to the fecal material. Such a method is counter to the conventional process of manufacturing a stool-based pharmaceutical product (e.g., an FMT product) that incorporates material (e.g., bacteria and/or non-living matter such as fiber) from a human stool, which conventional process rigorously screens donors and stool for the presence of pathogens, and adamantly selects against/rejects a donor or stool that comprises or is infected with a pathogen. In contrast, the present disclosure leverages pathogens in a donor stool to generate a vaccine for immunizing subjects against the pathogen. In an aspect, the pathogen in the donor stool is a corona virus, for example SARS-CoV-2. In an aspect, immunizing subjects with a composition comprising a vaccine against SARS-CoV-2 prevents a SARS-CoV-2 infection in the subject and/or the emergence of COVID-19 and/or one or more symptoms associated with COVID-19.

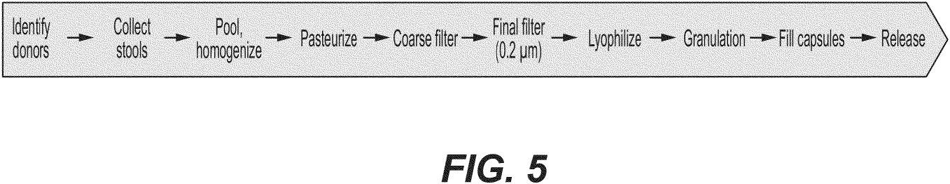

FIG. 5 shows a flowchart depicting exemplary steps that can be carried out to manufacture a composition described herein. These steps include the identification of a suitable donor comprising a pathogen against which an immune response is desired to be raised; the collection of separate stools from the donor; the pooling and homogenization of the collected stool; pasteurizing of the stool to inactivate the pathogen; filtering the homogenate using a sieve having relatively large pores (e.g., ~300 µm); filtering the first filtrate using a filter having pores of a size at or about 0.2 µm; lyophilizing the second filtrate; granularizing the lyophilized product; and encapsulating the granularized lyophilizate in a capsule for administration. In another aspect, an additional pasteurization step may be added prior to lyophilization.

Further disclosed herein are methods and compositions for detecting a pathogen in a human stool, selecting the stool based on the presence of the pathogen, inactivating the pathogen by heating the stool, homogenizing and filtering the stool to produce a fecal filtrate (e.g., before or after the pathogen is inactivated), and incorporating the fecal filtrate into a pharmaceutical composition. Such an assay can be used to detect a pathogen in either donor stool (i.e. to be formulated into a pharmaceutical composition disclosed herein) or patient or subject stool (e.g. to determine the presence or absence of a pathogen in stool of the subject administered the composition, for example to assess the efficacy of a vaccine administered to the subject).

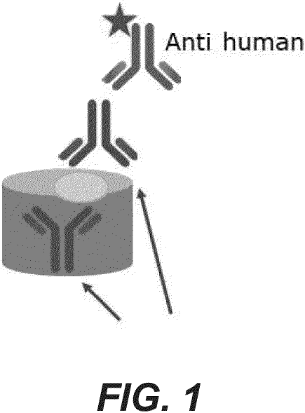

In an aspect, an immunoassay is performed to detect a pathogen in a stool. For example, the immunoassay can be an enzyme-linked immunosorbent assay (ELISA), which uses a solid-phase enzyme immunoassay to detect the presence of a ligand (e.g., pathogen antigen) in a liquid sample using antibodies direct to the ligand. The immunoassay (e.g., ELISA) can be used to detect an antigen from any pathogen that appears in stool (e.g., human stool). In an aspect, the pathogen is SARS-CoV-2. In one example, the ELISA assay is a “sandwich” ELISA or a “self-sandwich ELISA”. In a “sandwich” ELISA, the antigen of interest is quantified between two layers of antibodies, the capture antibody (e.g., rendered immobile by fixing to a surface) and the detection antibody. In certain cases the capture and detection antibodies target different epitopes of the antigen (e.g., pathogen antigen). In a “self-sandwich” ELISA (schematically depicted in FIG. 1), the same antibody is used for both capture and detection of the antigen. Typically such an antibody is polyclonal, unless an epitope targeted by a monoclonal antibody occurs in multiple locations on an antigen. In an aspect, an ELISA, sandwich ELISA, or self-sandwich ELISA employs an antibody that is exposed to a liquid preparation comprising human fecal material (e.g., diluted, homogenized and/or filtered stool). In the event that the pathogen is present in the stool, the antibody can bind to one or more epitopes of an antigen of the pathogen, which can be detected during the detection phase of the assay. In an aspect, an anti-SARS-CoV-2 antibody (e.g., from a polyclonal serum or a monoclonal anti-SARS-CoV-2 antibody) can be used to detect the presence of SARS-CoV-2 in the stool.

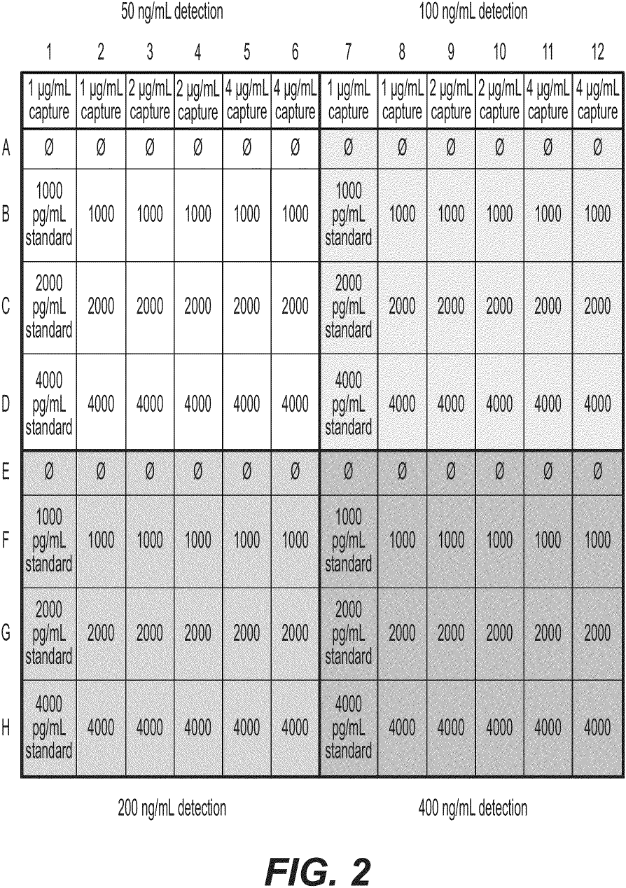

In another aspect, a receptor or protein of a viral host capable of binding an antigen of the virus can be exploited as an antibody to detect the pathogen in a stool of a donor or subject (as depicted schematically in FIG. 2). The protein or receptor (in FIG. 2, the ACE2 receptor of SARS-CoV-2) can be linked to a tag (e.g., His tag) which is bound by an anti-His antibody fixed to a surface. The protein or receptor can then be exposed to a liquid preparation comprising human fecal material (e.g., diluted, homogenized and/or filtered stool), allowing the protein or receptor to bind the viral antigen (here the S or spike protein of SARS-CoV-2). In the proof-of-concept schematic shown in FIG. 2, such S protein is linked to a mouse IgG Fc fragment, which can be detected using a standard immunoassay detection scheme (here an antimouse IgG Fc fragment fused to biotin, which is then exposed to streptavidin fused to HRP followed by addition of the HRP substrate). In an aspect, for detection of the S protein in a donor or patient stool, the S protein bound to the ACE2 receptor can be detected using a second antibody (e.g., polyclonal serum shown in FIG. 1) targeting a second epitope of the S protein, which itself can be detected using standard immunoassay detection schemes via an anti-human antibody linked to an immunoassay label (e.g. biotin).

In an aspect, a method of manufacturing a pharmaceutical composition (e.g., vaccine) comprises collecting a stool of a donor, testing the stool for the presence of a pathogen, and extracting fecal material from the stool (e.g., by homogenizing and filtering the stool) on the basis of detection of the pathogen in the stool. In an aspect, the donor is a candidate for carrying the pathogen on the basis that the donor exhibits or has recently exhibited symptoms of a disease or disorder associated with the pathogen.

In an aspect, a method of preventing an infection by a pathogen in a subject comprises administering to the subject a pharmaceutical composition described herein, and providing a certification to the subject that verifies such administration. In an aspect, such certification can comprise a “passport” that can be carried by the subject to verify to health or governmental authorities that the subject has been vaccinated.

In an aspect, a pharmaceutical composition comprises a bacterial mixture (e.g., as a fecal filtrate) comprising a non-viable fecal bacterial preparation, for example non-selected fecal bacteria and/or a substantially complete fecal microbiota of a stool or portion thereof (e.g., from a human donor). In an aspect, a substantially complete fecal microbiota, prior to removal of the bacterial viability (e.g., by application of heat), comprises viable bacterial cells from all or substantially all of the bacterial taxa represented among the viable bacterial cells in the stool from which the fecal microbiota was extracted. In an aspect, the relative abundance of viable bacterial cells from at least two of the taxa in the substantially complete fecal microbiota is proportional to the relative abundance of the viable cells from those taxa in the stool from which the fecal microbiota was extracted.

In one aspect, a fecal bacterial preparation comprises a donor’s entire or substantially complete fecal microbiota from a stool or portion thereof. In one aspect, a fecal bacterial preparation comprises a non-selective fecal microbiota. In another aspect, a fecal bacterial preparation comprises an isolated or purified population or community of live non-pathogenic fecal bacteria. In a further aspect, a fecal bacterial preparation comprises a non-selective and substantially complete fecal microbiota preparation from a single donor. In another aspect, a pharmaceutical composition used herein comprises a mixture of live, non-pathogenic, bacterial isolates and live, non-pathogenic, purified or extracted, fecal bacterial preparation.

In an aspect, the manufacture of a fecal filtrate (e.g., comprising a fecal bacterial preparation) involves a treatment selected from the group consisting of ethanol treatment, detergent treatment, heat treatment, irradiation, and sonication. In another aspect, the manufacture of a fecal filtrate involves no treatment selected from the group consisting of ethanol treatment, detergent treatment, heat treatment, irradiation, and sonication. In one aspect, the manufacture of a fecal filtrate involves a separation step selected from the group consisting of density gradients, filtration (e.g., sieves, nylon mesh), and chromatography. In another aspect, the manufacture of a fecal filtrate involves no separation step selected from the group consisting of density gradients, filtration (e.g., sieves, nylon mesh), and chromatography. In another aspect, a fecal filtrate comprises an entire or substantially entire fecal microbiota from a stool sample of a subject. In another aspect, a pharmaceutical composition administered herein comprises a fecal microbiota substantially free of donor eukaryotic cells.

In an aspect, a pharmaceutical composition provided or administered herein comprises a fecal bacterial preparation comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In aspect the fecal bacterial preparation comprises non-viable fecal bacteria. In another aspect, the fecal bacterial preparation does not comprise viable fecal bacteria. In another aspect, the pharmaceutical composition comprises a non-viable or sterilized fecal microbiota. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora. In a further aspect, a pharmaceutical composition comprises a preparation of non-viable flora that in taxonomic content resembles a normal healthy human fecal flora.

In a further aspect, a pharmaceutical composition comprises fecal bacteria from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition comprises non-viable fecal bacteria.

In an aspect, a pharmaceutical composition provided or administered herein comprises a fecal bacterial preparation comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semipurified or purified formulation.

In an aspect, a fecal bacterial preparation included in a pharmaceutical composition comprises highly refined or purified fecal flora, e.g., substantially free of non-floral fecal material. In an aspect, a fecal microbiota (comprising a fecal bacterial preparation) harvested from a donor can be further processed, e.g., to undergo microfiltration before, after, or before and after sieving. In another aspect, a highly purified fecal microbiota product is ultra-filtrated to remove large molecules but retain the therapeutic microflora, e.g., bacteria or non-viable bacteria.

In another aspect, a fecal bacterial preparation in a pharmaceutical composition used herein comprises or consists essentially of a substantially isolated or a purified fecal flora or entire (or substantially entire) microbiota that is (or comprises) an isolate of fecal flora that is at least about 90%, 91 %, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal bacterial preparation included in a pharmaceutical composition comprises a donor’s substantially entire or non-selected fecal microbiota. In another aspect, the fecal microbiota in a pharmaceutical composition comprises no antibiotic resistant population. In another aspect, a pharmaceutical composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter’s donor). In another aspect, a pharmaceutical composition comprises a non-viable fecal microbiota and is largely free of extraneous matter.

In an aspect, a fecal bacterial preparation included in a pharmaceutical composition is derived from a disease-screened stool or portion thereof of a human donor (e.g., allowing selection for a donor infected by a virus which is the target of a vaccine described herein). In an aspect, a stool or portion thereof does not include an antibiotic resistant population. For example, a composition can comprise a preparation of viable flora which can in proportional content, resemble normal healthy human fecal flora which does not include antibiotic resistant populations.

In one aspect, a fecal bacterial preparation comprises one or more, two or more, three or more, four or more, or five or more live (i.e., prior to treatment to remove viability) fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation comprises one or more, two or more, three or more, four or more, or five or more live fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, , Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In one aspect, a fecal microbiota preparation (e.g. fecal bacterial preparation) described and used here lacks or is substantially devoid of one or more, two or more, three or more, four or more, or five or more live fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation lacks or is substantially devoid of one or more, two or more, three or more, four or more, or five or live more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale , Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, , Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In an aspect, a pharmaceutical composition comprises non-viable Clostridium and a plurality of non-viable microorganisms from one or more genera selected from the group consisting of Collinsella, Coprococcus, Dorea, Eubacterium, and Ruminococcus. In another aspect, a pharmaceutical composition comprises a plurality of non-viable microorganisms from one or more genera selected from the group consisting of Clostridium, Collinsella, Coprococcus, Dorea, Eubacterium, and Ruminococcus.

In an aspect, a fecal bacterial preparation for incorporation into a pharmaceutical composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a pharmaceutical composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides uniformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a pharmaceutical composition comprises viable non-pathogenic Clostridium and a plurality of viable non-pathogenic microorganisms from one or more genera selected from the group consisting of Collinsella, Coprococcus, Dorea, Eubacterium, and Ruminococcus. In another aspect, a pharmaceutical composition comprises a plurality of viable non-pathogenic microorganisms from one or more genera selected from the group consisting of Clostridium, Collinsella, Coprococcus, Dorea, Eubacterium, and Ruminococcus.

In an aspect, a pharmaceutical composition comprises two or more genera selected from the group consisting of Collinsella, Coprococcus, Dorea, Eubacterium, and Ruminococcus. In another aspect, a pharmaceutical composition comprises two or more genera selected from the group consisting of Coprococcus, Dorea, Eubacterium, and Ruminococcus. In a further aspect, a pharmaceutical composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In an aspect, a pharmaceutical composition comprises non-viable bacteria from two or more genera selected from the group consisting of Collinsella, Coprococcus, Dorea, Eubacterium, and Ruminococcus. In another aspect, a pharmaceutical composition comprises non-viable bacteria from two or more genera selected from the group consisting of Coprococcus, Dorea, Eubacterium, and Ruminococcus. In a further aspect, a pharmaceutical composition comprises non-viable bacteria from one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In an aspect, a pharmaceutical composition described herein does not contain any spores. In an aspect, a pharmaceutical composition described herein does not contain any viable spores.

In an aspect, a fecal bacterial preparation described herein, prior to inactivation to remove viability of cells, comprises viable cells from 100% of the viable bacterial taxa represented in the stool from which the fecal bacteria were derived. In an aspect, a fecal bacterial preparation described herein comprises viable cells from at least 99% of the viable bacterial taxa represented in the stool from which the fecal bacteria were derived. In an aspect, a fecal bacterial preparation described herein comprises viable cells from at least 98% of the viable bacterial taxa represented in the stool from which the fecal bacteria were derived. In an aspect, a fecal bacterial preparation described herein comprises viable cells from at least 97% of the viable bacterial taxa represented in the stool from which the fecal bacteria were derived. In an aspect, a fecal bacterial preparation described herein comprises viable cells from 96% of the viable bacterial taxa represented in the stool from which the fecal bacteria were derived. In an aspect, a fecal bacterial preparation described herein comprises viable cells from at least 95, 94, 93, 92, 91, 90, 89, 88, 87, 85, 84, 83, 82, 81, 80, 75, 70, 65, 60, 55, 50, 45, or 40% of the viable bacterial taxa represented in the stool from which the fecal bacteria were derived.

In an aspect, a non-viable fecal bacterial preparation described herein comprises non-viable cells from 100% of the bacterial taxa represented in the stool from which the fecal bacteria were derived. In an aspect, a non-viable fecal bacterial preparation described herein comprises non-viable cells from at least 99% of the bacterial taxa represented in the stool from which the fecal bacteria were derived. In an aspect, a non-viable fecal bacterial preparation described herein comprises non-viable cells from at least 98% of the bacterial taxa represented in the stool from which the fecal bacteria were derived. In an aspect, a non-viable fecal bacterial preparation described herein comprises non-viable cells from at least 97% of the bacterial taxa represented in the stool from which the fecal bacteria were derived. In an aspect, a non-viable fecal bacterial preparation described herein comprises non-viable cells from 96% of the bacterial taxa represented in the stool from which the fecal bacteria were derived. In an aspect, a non-viable fecal bacterial preparation described herein comprises non-viable cells from at least 95, 94, 93, 92, 91, 90, 89, 88, 87, 85, 84, 83, 82, 81, 80, 75, 70, 65, 60, 55, 50, 45, or 40% of the bacterial taxa represented in the stool from which the fecal bacteria were derived.

In an aspect, a pharmaceutical composition disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary pharmaceutical composition comprising a sterile fecal filtrate comprises starting material from a donor from a defined donor pool, where this donor contributes a stool that is homogenized and centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material can be separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation can in some aspects be filtered progressively in a filtering, e.g., a Millipore filtering or equivalent systems, to produce a liquid which is finely filtered through an about 0.22 micron filter. This removes all particulate matter including all living matter, including bacteria and some intact viruses. The product then is sterile, but the aim is to (i) remove intact viruses but keep portions thereof, e.g., viral peptides, and (ii) remove the bacteria but keep their secretions, which can potentially exhibit adjuvant or antimicrobial activity, for example antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition comprises reconstituted non-viable fecal flora consisting essentially of a combination of a purified non-viable fecal microbiota (comprising a non-viable fecal bacterial preparation) and a non-cellular fecal filtrate. In another aspect, a pharmaceutical composition comprises reconstituted fecal flora consisting essentially of a combination of a purified fecal microbiota (comprising a fecal bacterial preparation) and a non-cellular (sterile) fecal filtrate. In another aspect, a pharmaceutical composition comprises a purified fecal microbiota (comprising a fecal bacterial preparation) supplemented with one or more non-cellular non-particulate fecal components. In one aspect, a pharmaceutical composition comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules (e.g., adjuvants) selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a pharmaceutical composition comprises both a fecal bacterial preparation, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products (e.g., adjuvants) such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

The pharmaceutical compositions described here can comprise microbes, e.g., non-viable bacteria, derived from a stool of a donor, e.g. a healthy human donor. In an aspect, a composition incorporates a non-viable fecal bacterial preparation derived from all or a portion of a fecal microbiota of a stool or portion thereof of a healthy human donor.

In one aspect, an exemplary fecal microbiota for use in preparing a composition described herein (e.g., comprising a non-viable fecal bacterial preparation) comprises starting material from a human donor. In another aspect, an exemplary fecal microbiota comprises material from one or more healthy human donors. In yet another aspect, an exemplary fecal microbiota comprises starting material from a pool of known, defined donors. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years).

In an aspect, a fecal donor is prescreened for its fecal microbiome profile. In another aspect, a fecal donor is selected for the presence of one or more fecal bacterial class, family, genus, or species in the donor’s stool. In another aspect, a fecal donor is selected for the presence of one or more fecal bacterial class, family, genus, species or strain in the donor’s stool at a level above a threshold abundance. In an aspect, a fecal donor can be selected on the basis of the presence or threshold abundance of one or more bacterial genera in the stool of the donor selected from the group consisting of Lactobacillus, Bifidobacterium, Streptococcus, Prevotella, Desulfovibrio, and a combination thereof. In an aspect, a fecal donor can be selected on the basis of the presence or threshold abundance of one or more bacterial genera in the stool of the donor selected from the group consisting of Clostridium, Bacteroides, Eggerthella, Bifidobacterium, Prevotella, and Desulfovibrio and a combination thereof. In an aspect, a fecal donor can be selected on the basis of the presence or threshold abundance of one or more bacterial taxa in the stool of the donor selected from the group consisting of Prevotella, Coprococcus, Prevotellaceae, and Veillonellaceae, and a combination thereof. In an aspect, a fecal donor can be selected on the basis of the presence or threshold abundance of one or more bacterial genera selected from the group consisting of Lactobacillus, Bifidobacterium, Streptococcus, and a combination thereof. In an aspect, a fecal donor can be selected on the basis of the presence or threshold abundance in the stool of the donor of the genus Lactobacillus.

In an aspect, a stool or portion thereof (also referred to herein as a stool sample) can be selected as a source of a fecal bacterial preparation for incorporation into a pharmaceutical composition on the basis of the presence or threshold abundance of one or more bacterial class, family, genus, species or strain in the stool sample. In an aspect, the stool sample can be selected on the basis of the presence or threshold abundance of a member of a bacterial genus selected from the group consisting of Lactobacillus, Bifidobacterium, Streptococcus, and a combination thereof.

In one aspect, exemplary stool for use in preparing a composition described herein (e.g., comprising a bacterial mixture comprising one or more of a fecal bacterial preparation and at least one bacterial isolate) is collected from a single human donor infected with a pathogen that is a target of a vaccine described herein. In another aspect, exemplary stool comprises starting material from a pool of known, defined donors, e.g. each being infected with a pathogen that is a target of a vaccine described herein. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years).

In an aspect, a fecal donor is prescreened for its fecal microbiome profile. In another aspect, a fecal donor is selected based on the presence of one or more pathogens in the donor and/or in a stool of the donor. In another aspect, a fecal donor is selected for the presence of one or more fecal bacterial class, family, genus, or species in the donor’s stool. In another aspect, a fecal donor is selected for the presence of one or more fecal bacterial class, family, genus, species or strain in the donor’s stool at a level above a threshold abundance. In an aspect, a fecal donor can be selected on the basis of the presence or threshold abundance of one or more bacterial genera in the stool of the donor selected from the group consisting of Lactobacillus, Bifidobacterium, Streptococcus, Prevotella, Desulfovibrio, and a combination thereof. In an aspect, a fecal donor can be selected on the basis of the presence or threshold abundance of one or more bacterial genera in the stool of the donor selected from the group consisting of Clostridium, Bacteroides, Eggerthella, Bifidobacterium, Prevotella, and Desulfovibrio and a combination thereof. In an aspect, a fecal donor can be selected on the basis of the presence or threshold abundance of one or more bacterial taxa in the stool of the donor selected from the group consisting of Prevotella, Coprococcus, Prevotellaceae, and Veillonellaceae, and a combination thereof. In an aspect, a fecal donor can be selected on the basis of the presence or threshold abundance of one or more bacterial genera selected from the group consisting of Lactobacillus, Bifidobacterium, Streptococcus, and a combination thereof. In an aspect, a fecal donor can be selected on the basis of the presence or threshold abundance in the stool of the donor of the genus Lactobacillus. In an aspect, the fecal donor can be selected on the basis of the presence or threshold abundance of Lactobacillus reuteri in the stool of the donor.

In an aspect, a stool sample can be selected as a source of a fecal bacterial preparation for incorporation into a pharmaceutical composition on the basis of the presence or threshold abundance of one or more bacterial class, family, genus, species or strain in the stool sample. In an aspect, the stool sample can be selected on the basis of the presence or threshold abundance of a member of a bacterial genus selected from the group consisting of Lactobacillus, Bifidobacterium, Streptococcus, and a combination thereof. In an aspect, the stool sample can be selected on the basis of the presence or a threshold abundance of Lactobacillus reuteri in the stool sample.

In an aspect, a carefully screened donor undergoes a complete medical history and physical exam. Exclusion criteria can comprise one or more of the following:
1. History of irritable bowel syndrome. Specific symptoms can include frequent abdominal cramps, excessive gas, bloating, abdominal distension, fecal urgency, diarrhea, constipation.
2. History of inflammatory bowel disease such as Crohn’s disease, ulcerative colitis, microscopic colitis.
3. Chronic diarrhea.
4. Chronic constipation or use of laxatives.
5. History of gastrointestinal malignancy or known colon polyposis.
6. History of any abdominal surgery, e.g., gastric bypass, intestinal resection, appendectomy, cholecystectomy, etc.
7. Use of Probiotics or any other over the counter aids used by the potential donor for purpose of regulating digestion. Yogurt and kefir products are allowed if taken merely as food rather than nutritional supplements.
8. Antibiotics for any indication within the preceding 6 months.
9. Any prescribed immunosuppressive or anti-neoplastic medications.
10. Metabolic Syndrome, established or emerging. Criteria used for definition here are stricter than any established criteria. These include history of increased blood pressure, history of diabetes or glucose intolerance.
11. Known systemic autoimmunity, e.g., connective tissue disease, multiple sclerosis.
12. Known atopic diseases including asthma or eczema.
13. Chronic pain syndromes including fibromyalgia, chronic fatigue syndrome.
14. Ongoing (even if intermittent) use of any prescribed medications, including inhalers or topical creams and ointments.
15. Neurologic, neurodevelopmental, and neurodegenerative disorders including autism, Parkinson’s disease.
16. General. Body mass index > 26 kg/ m2, central obesity defined by waste:hip ratio > 0.85 (male) and > 0.80 (female).
17. Blood pressure > 135 mmHg systolic and > 85 mmHg diastolic.
18. Skin — presence of a rash, tattoos or body piercing placed within a year, or jaundice
19. Enlarged lymph nodes.
20. Wheezing on auscultation.
21. Hepatomegaly or stigmata of liver disease.
22. Swollen or tender joints. Muscle weakness.
23. Abnormal neurologic examination.
24. Positive stool Clostridium difficile toxin B tested by PCR.
25. Positive stool cultures for any of the routine pathogens including Salmonella, Shigella, Yersinia, Campylobacter, E. coli 0157:H7.
26. Abnormal ova and parasites examination.
27. Positive Giardia, Cryptosporidium, or Helicobacter pylori antigens.
28. Positive screening for any undesired viral illnesses, including HIV 1 and 2, Viral Hepatitis A IgM, Hepatitis surface antigen and core Ab.
29. Abnormal RPR (screen for syphilis).
30. Any abnormal liver function tests including alkaline phosphatase, aspartate aminotransaminase, alanine aminotransferase.
31. Raised serum triglycerides > 150 mg/Dl
32. HDL cholesterol < 40 mg/dL (males) and < 50 mg/dL (females)
33. High sensitivity CRP > 2.4 mg/L
34. Raised fasting plasma glucose (> 100 mg/dL)

In some aspects, additional exclusion criteria comprises known viral infection with Hepatitis B, C, or HIV. In another aspect, donors are excluded if they exhibit high risk behaviors including sex for drugs or money, men who have sex with men, more than one sexual partner in the preceding 12 months, any past use of intravenous drugs or intranasal cocaine, history of incarceration. In another aspect, donors are excluded if they have a current communicable disease, e.g., upper respiratory viral infection.

In one aspect, provided herein is a process for collecting and processing a stool sample to give rise to a fecal bacterial preparation (such preparation to be treated with a sterilizing agent to produce a non-viable fecal bacterial preparation). The process can comprise first collecting a stool sample from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No: 7,541,091. In another aspect, the container itself is made of an oxygen scavenging material, e.g., oxygen scavenging iron, e.g., as described by O2BLOCKTM, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222 (hereby incorporated herein by reference in its entirety), describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554 (hereby incorporated herein by reference in its entirety), describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231 (hereby incorporated herein by reference in its entirety), describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, stool collected from a donor is held in an aesthetically acceptable container that will not leak nor smell yet maintain an anaerobic environment. In one aspect, the container is sterile before receiving the fecal flora.

In one aspect, a stool sample provided herein is maintained at room temperature during most or all of its transportation and/or storage at e.g., a “stool bank”. For example, once delivered to a “processing stool bank” it is stored at ambient temperature, e.g., room temperature. In one aspect, stabilizing agents, such as glycerol, are added to the harvested and/or stored material.

In one aspect, the stool is tested for various pathogens. In one aspect, once cleared of infective agents, a stool sample is homogenized and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLARTM.

In one aspect, the stool is tested for the presence of a pathogen that is the target of a vaccine described herein, as noted above. In one aspect, where a test reveals the presence of the pathogen in the stool, or the presence of the pathogen (e.g., virus) above a threshold titer, a stool sample is processed, e.g., pasteurized, homogenized and filtered to produce a fecal filtrate. In another aspect, a stool collected from a donor can be heated (e.g., pasteurized) in its raw form. For example, the stool can be heated to about 60° C. for 10 hours in a sealed container. Such pasteurization of raw stool has the benefit of inactivating pathogen in the stool to reduce the likelihood of transmission of the virus during processing. Following such pasteurization, the stool can be subjected to downstream processing such as filtering to produce a preparation (e.g. fecal filtrate) for incorporation into a pharmaceutical composition.

In one aspect, the stool is subject to homogenization by for example, mixing, agitating, stirring or shaking. In certain aspects, a stool sample is diluted with a homogenization buffer prior to homogenization. A homogenization buffer can, for example, contain a cryoprotectant (e.g., trehalose), an antioxidant or reducing agent (e.g., cysteine), and a buffer (e.g., 0.25X PBS at pH 7.4). In other aspects, a homogenization buffer does not comprise a cryoprotectant or antioxidant.

In one aspect, to separate the non-bacterial components from the fecal microbiota, the stool can be homogenized and filtered from rough particulate matter. In one aspect, the microscopic fiber/nonliving matter is then separated from the bacteria. Several methods can be used, including e.g., recurrent filtration with filter sizes, e.g., progressively coming down to the size of a typical bacterium.

In one aspect, different filters are used to isolate bacterial sp., or a technique as used by Williams in WO 2011/033310A1 (hereby incorporated herein by reference in its entirety), which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

### Pharmaceutical Compositions, Formulations, and Administration

Described herein are pharmaceutical compositions comprising a bacterial mixture comprising a non-viable fecal bacterial preparation in various formulations.

Described herein are pharmaceutical compositions comprising one or more microbial (e.g. pathogen) antigens along with fecal material derived from a stool of human donor. In an aspect, the pharmaceutical composition comprises one or more viral antigens in combination with a fecal-derived adjuvant in various formulations. Any pharmaceutical composition described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with the compositions described herein in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can be incorporated into tablets, drenches, boluses, capsules, premixes or patches. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid pre-formulation composition containing a homogeneous mixture of a composition described herein. When referring to these pre-formulation compositions as homogeneous, it is meant that the active ingredient is dispersed evenly throughout the composition so that the composition can be readily subdivided into equally effective unit dosage forms such as tablets, pills and capsules. This solid pre-formulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ CFUs). A pharmaceutical composition described herein can be flavored.

In an aspect, a pharmaceutical composition comprising a non-viable fecal bacterial preparation described herein (and optionally one or more additional therapeutic agents) is formulated as a composition adapted for a mode of administration described herein.

In various aspects, the administration of a pharmaceutical composition is any one of oral, intravenous, intraperitoneal, and parenteral. For example, routes of administration include, but are not limited to, oral, intraperitoneal, intravenous, intramuscular, or rectally. In various aspects, the administration of the pharmaceutical compositions is oral, naso-gastric, antegrade gastrointestinal, retrograde gastrointestinal, endoscopic, or enemic. In another aspect, the pharmaceutical compositions are formulated for systemic or local delivery. In an aspect, administration is systemic. In another aspect, it may be desirable to administer locally to the area in need of treatment.

In an aspect, a pharmaceutical composition described herein can be formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide sustained delivery over an extended period of time. Selectively permeable membranes surrounding an osmotically active agent are also suitable for orally administered compositions. In these latter platforms, fluid from the environment surrounding the capsule is imbibed by a driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In an aspect, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various aspects, a pharmaceutical composition is formulated as a solid dosage form such as a tablet, dispersible powder, granule, or capsule. In an aspect, the pharmaceutical composition is formulated as a capsule. In another aspect, the pharmaceutical composition is formulated as a tablet. In yet another aspect, the pharmaceutical composition is formulated as a soft-gel capsule. In a further aspect, the pharmaceutical composition is formulated as a gelatin capsule.

In an aspect, a pharmaceutical composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a pharmaceutical composition described herein is formulated in in the form of microcapsules. Microencapsulation is the coating of a liquid or solid with a protective wall material that inhibits volatilization and protects against chemical deterioration. The solid or liquid contained within the wall material is known as the core, and the complete microencapsulated particle is known as a microcapsule. Wall materials that can be used here include gum arabic, carboxymethyl cellulose, alginate, gelatin, whey protein, sodium caseinate, and soy protein.

In various aspects, formulations can additionally comprise a pharmaceutically acceptable carrier or excipient. As one skilled in the art will recognize, the formulations can be in any suitable form appropriate for the desired use and route of administration.

Microencapsulation can be performed with a microencapsulation device, including microfluidic droplet generation or encapsulation devices. An exemplary microencapsulation device is described, for example, in U.S. Pat. No. 7,482,152, hereby incorporated herein by reference in its entirety. Microcapsules can comprise one or more stabilizers or gelling agents, which can be used to stabilize a microcapsule or emulsion. Stabilizers or gelling agents can include but are not limited to alginate (also algin or alginic acid) and agar. Alginate can be used in a variety of forms, including but not limited to inorganic salts such as sodium alginate, potassium alginate, calcium alginate, and combinations thereof. Alginate can be derived from sources such as seaweed (e.g., Macrocystis pyrifera, Ascophyllum nodosum, Laminaria spp.) or bacteria (e.g., Pseudomonas spp., Azotobacter spp.). Cross-linking agents or solutions, such as calcium chloride, can be used to stabilize or gel microcapsules. In an aspect, alginate-based microcapsules are made and used according to US20160317583. In an aspect, an alginate polymer has a concentration of about 2.5% (w/v) wherein the microcapsule comprises an additional sequential external surface coating of poly-L-lysine. In a further aspect, microcapsule comprises a divalent cation from the group of calcium or barium or a mixture of calcium and barium to crosslink the alginate polymer into a microcapsule.

Microcapsules can be characterized by a size (e.g., a diameter). The microcapsule size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be less than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be from about 0.05 to about 1 millimeters. The size distribution in a population of microcapsules can be homogeneous or substantially homogeneous. For example, a population of microcapsules can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

In various aspects, formulations can additionally comprise a pharmaceutically acceptable carrier or excipient. As one skilled in the art will recognize, the formulations can be in any suitable form appropriate for the desired use and route of administration.

In some dosage forms, a pharmaceutical composition described herein is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In an aspect, a pharmaceutical composition is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof.

In an aspect, a pharmaceutical composition is combined with other adjuvants such as antacids to dampen inactivation of bacteria present in the composition in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In one aspect, a pharmaceutical composition as described herein is administered via a syringe. The term “syringe” refers to a reservoir (that holds a liquid) and a plunger. A syringe may be used in combination with a needle. In some aspects, the term “syringe” is also used to refer to the entire reservoir/plunger/needle combination. Syringes come in a variety of sizes and types. By way of non-limiting examples, the syringe can be a Luer lock syringe, a normal slip tip or Luer slip syringe, an eccentric tip syringe, an insulin syringe, etc. In one aspect, the syringe can be a retractable syringe. In one aspect, the syringe can be a pre-fillable syringe. In one aspect, the syringe is made of plastic, glass, or stainless steel. In one aspect the syringe is disposable. In one aspect the syringe is reusable. The syringe may be used with needles of different length and gauge.

In an aspect, a fecal filtrate described herein is dry, e.g., the filtrate is lyophilized and/or comprises dry binders, fillers, and dispersants. Alternately, the fecal filtrate can be aqueous, e.g., when it comprises non-dry binders, fillers, and dispersants.

In an aspect, a fecal filtrate described herein can be subject to lyophilization. As used herein, “lyophilization” or “freeze drying” refers to the process of drying a material by first freezing it and then encouraging the ice within it to sublimate in a vacuum environment. Additional relevant teachings are disclosed in WO 2007122374, which is hereby incorporated herein by reference in its entirety.

In an aspect, a bacterial mixture (comprising a non-viable fecal bacterial preparation) described herein can be subject to lyophilization.

In one aspect, a lyophilized formulation further comprising a reducing agent and/or antioxidant. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, glutathione, methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a fecal filtrate comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In an aspect of the present disclosure, a cryoprotectant can be selected from the group comprising 5% sucrose; 10% sucrose; 10% skim milk; 10% trehalose with 2.5% sucrose; 5% trehalose with 2.5% sucrose; 5% mannitol; 5% mannitol with 0.1% polysorbate 80; 10% mannitol; 10% mannitol with 0.1% polysorbate 80; 5% trehalose; 5% trehalose with 0.1% polysorbate 80; 10% trehalose; and 10% trehalose with 0.1% polysorbate 80.

In an aspect, a fecal filtrate comprises a lyoprotectant. As used herein, a “lyoprotectant” refers to a substance that is added to a formulation in order to protect an active ingredient during the stage of a lyophilization (also known as freeze-drying). In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In an aspect, a lyoprotectant is a non-reducing sugar, such as trehalose or sucrose. In an aspect, a cryoprotectant or a lyoprotectant consists essentially of, or consists of, one or more substances mentioned in this paragraph and the paragraph above.

In an aspect, a cryoprotectant or a lyoprotectant comprise an intracellular agent, e.g., DMSO, glycerol, or PEG, which penetrates inside the cell preventing the formation of ice crystals that could result in membrane rupture. In an aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In an aspect, a lyophilized formulation comprises trehalose. In an aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In an aspect, a lyophilized formulation comprises at least 2%, 3%, 4%, 5%, 6%, 7%, 8%, 9%, 10%, or 15% trehalose. In an aspect, a lyophilized formulation comprises at most 2%, 3%, 4%, 5%, 6%, 7%, 8%, 9%, 10%, or 15% trehalose. In another aspect, a lyophilized formulation comprises about 5% trehalose. In another aspect, a lyophilized formulation comprises trehalose and sucrose. In another aspect, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In an aspect, a pharmaceutical composition provided here, after at least 12 weeks of storage at ambient temperature or lower, is effective for vaccinating a subject. In an aspect, a pharmaceutical composition remains effective after at least 4, 8, 10, 16, 20, 24, 30, 40, 50, 60, 70, 80 or 100 weeks of storage at ambient temperature or lower.

In an aspect, a pharmaceutical composition described herein can be lyophilized or freeze dried and stored at ambient temperatures (e.g., room temperature), at a freezing temperature, or at between about 2oC and 8oC. In an aspect, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying or lyophilization, in aspects where a fecal filtrate comprises bacteria, the bacteria can be present in a liquid that will prevent bursting of cells on thawing. This can include various stabilizers, e.g., glycerol and appropriate buffers, and/or ethylene glycol. In an aspect, the cryoprotecting process uses final concentrations of stabilizer(s) of between about 10% and 80%, 20% and 70%, 30% and 60%, or 40% and 50%, depending on the stabilizer(s) used; in an aspect, this helps stabilize proteins by preventing formation of ice crystals that would otherwise destroy protein structures.

In an aspect, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In an aspect, the pharmaceutical composition described herein are single or double encapsulated. For example, a lyophilized fecal filtrate comprising one or more pathogen antigens can be double encapsulated in hypromellose capsules and delivered orally to a subject.

In an aspect, one or more additional therapeutic agents can be included in a pharmaceutical composition, and encapsulated by the capsule.

In an aspect, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm2 and 6 mg/cm2, e.g., 3 mg/cm2. After completion of this step, the capsules are set to dry, e.g., between about 8 hours and 24 hours. After drying, exemplary capsules are weighed to calculate weight gain from the exterior enteric coating. Capsules can be inspected for irregularities.

In an aspect, EUDRAGIT® S100 (poly(methacrylic acid, methylmethacrylate)), starch, triethyl citrate, and PlasACRYL® T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further processing, e.g., filling with a formulation or composition described herein.

In an aspect, it may be desirable to provide an amount of the formulation to a capsule’s cap in addition to providing the composition in the capsule’s body. In this aspect, more of the composition will be included in a capsule and/or less air will be contained in a closed capsule.

In an aspect, the interior surface of a capsule comprises an internal coating, for example an internal coating that is water insoluble.

Any of the above-described compositions and materials (e.g., fecal filtrates, preparations of uncultured bacteria, fecal bacterial preparations (viable or non-viable) inner coatings, capsules, and outer coatings) can be combined into a pharmaceutical composition described herein. A skilled artisan would know how to select an inner coating; capsule, and outer coating according to his/her present need, which could be based, for example, on a specific species of bacteria incorporated into the composition and/or the desired delivery location in a subject (e.g., in the colon or small intestine, including the ileum, jejunum or duodenum).

In an aspect, during the manufacture of a pharmaceutical composition, a pharmaceutically-acceptable cryoprotectant, lyoprotectant, binder, disintegrant, filler, preservative, acid suppressant, antacid, H2 antagonist, and proton pump inhibitor, or combination thereof can be mixed into the pharmaceutical composition (e.g., comprising a fecal filtrate) to promote desirable properties.

In an aspect, the pharmaceutical composition comprises a surface active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some aspects, compositions can comprise one or more surfactants including, but not limited to, sodium lauryl sulfate, polysorbate 20, polysorbate 40, polysorbate 60, polysorbate 80, and triethyl citrate.

In an aspect, the pharmaceutical composition comprises pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

In another aspect, the pharmaceutical composition comprises one or more application solvents. Some of the more common solvents that can be used to apply, for example, a delayed-release coating composition include isopropyl alcohol, acetone, methylene chloride and the like.

In yet another aspect, the pharmaceutical composition comprises one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the orally administered compositions can also include adjuvants such as sweetening, flavoring, and perfuming agents.

Various methods can be used to formulate and/or deliver a pharmaceutical composition (e.g., comprising a fecal filtrate) described herein to a location of interest. For example, the pharmaceutical compositions can be formulated for delivery to the GI tract. The GI tract includes organs of the digestive system such as mouth, esophagus, stomach, small intestine, duodenum, jejunum, ileum, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery of one or more active agents to one or more of the stomach, small intestine, large intestine and rectum, or any subsection thereof (e.g. duodenum, jejunum and ileum, colon transversum, colon descendens, colon ascendens, colon sigmoidenum and cecum). In some aspects, the compositions described herein can be formulated to deliver to the upper or lower GI tract. In an aspect, a composition can be administered to a subject, by, for example, directly or indirectly contacting the mucosal tissues of the GI tract with the composition.

In various aspects, the administration of the pharmaceutical compositions is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, a gelatin-based chewable (e.g., a gummy), flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid. In another aspect, a pharmaceutical composition comprises a liquid non-viable fecal bacterial preparation. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various aspects, provided herein are modified-release formulations comprising a bacterial mixture (e.g., comprising a non-viable fecal bacterial preparation), wherein the formulation releases a substantial amount of the bacterial mixture (and optionally additional therapeutic agents) into one or more regions of the GI tract.

In various aspects, provided herein are modified-release compositions comprising formulations comprising a pathogen antigen and fecal-derived adjuvant (e.g., comprising a sterile filtrate or bacterial cells in combination with an inactive pathogen or portion thereof), wherein the composition releases a substantial amount of the formulation into one or more regions of the GI tract. For example, the composition can release at least about 60% of the formulation after the stomach and into one or more regions of the GI tract.

In various aspects, the modified-release composition can release at least 60% of the formulation after the stomach into one or more regions of the intestine. For example, the modified-release composition releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the formulation in the intestines.

In various aspects, the modified-release composition can release at least 60% of the formulation in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the formulation in the small intestine (e.g., one or more of duodenum, jejunum, ileum, and ileocecaljunction).

In various aspects, the modified-release formulation can release at least 60% of the formulation in the large intestine. For example, the modified-release composition releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the formulation in the large intestine (e.g., one or more of cecum, ascending, transverse, descending or sigmoid portions of the colon, and rectum).

In some aspects, the pharmaceutical composition is formulated for release in the stomach. In other aspects, the pharmaceutical composition is formulated so as to not substantially release the pathogen antigen/fecal derived adjuvant formulation in the stomach.

In certain aspects, the modified-release composition releases the formulation at a specific pH. For example, in some aspects, the modified-release composition is substantially stable in an acidic environment and substantially unstable (e.g., dissolves rapidly or is physically unstable) in a near neutral to alkaline environment. In some aspects, stability is indicative of not substantially releasing while instability is indicative of substantially releasing. For example, in some aspects, the modified-release composition is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some aspects, the present compositions are stable in lower pH areas and therefore do not substantially release in, for example, the stomach. In some aspects, the modified-release composition is substantially stable at a pH of about 1 to about 4 or lower and substantially unstable at pH values that are greater. In these aspects, the modified-release composition does not substantially release in the stomach. In these aspects, the modified-release composition substantially releases in the small intestine (e.g. one or more of the duodenum, jejunum, and ileum) and/or large intestine (e.g. one or more of the cecum, ascending colon, transverse colon, descending colon, and sigmoid colon). In some aspects, the modified-release composition is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these aspects, the modified-release composition substantially releases in the large intestine (e.g. one or more of the cecum, ascending colon, transverse colon, descending colon, and sigmoid colon). In various aspects, the pH values recited herein can be adjusted as known in the art to account for the state of the subject, e.g. whether in a fasting or postprandial state.

In various aspects, formulations can take the form of those described in one or more of U.S. Pat. Nos. 8,535,713 and 8,9117,77 and U.S. Pat. Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and U.S. Pat. Publication Nos. 2014/0302132; 2014/0227357; 20140088202; 20130287842; 2013/0295188; 2013/0307962; and 20130184290, the contents of which are hereby incorporated by reference in their entirety.

In some aspects, the modified-release composition is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some aspects, the modified-release composition is stable in gastric fluid or stable in acidic environments. These modified-release compositions release about 30% or less by weight of the formulation in the modified-release composition in gastric fluid with a pH of about 4 to about 5 or less, or simulated gastric fluid with a pH of about 4 to about 5 or less, in about 15, or about 30, or about 45, or about 60, or about 90 minutes. Modified-release compositions of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release composition in gastric fluid with a pH of 4-5, or less or simulated gastric fluid with a pH of 4-5 or less, in about 15, or about 30, or about 45, or about 60, or about 90 minutes. Modified-release compositions can release about 1%, about 2%, about 3%, about 4%, about 5%, about 6%, about 7%, about 8%, about 9%, or about 10% by weight of the total composition in the modified-release composition in gastric fluid with a pH of 5 or less, or simulated gastric fluid with a pH of 5 or less, in about 15, or about 30, or about 45, or about 60, or about 90 minutes.

In some aspects, the modified-release composition is unstable in intestinal fluid. These modified-release compositions release about 70% or more by weight of the formulation in the modified-release composition in intestinal fluid or simulated intestinal fluid in about 15, or about 30, or about 45, or about 60, or about 90 minutes. In some aspects, the modified-release composition is unstable in near neutral to alkaline environments. These modified-release compositions release about 70% or more by weight of the formulation in the modified-release composition in intestinal fluid with a pH of about 4-5 or greater, or simulated intestinal fluid with a pH of about 4-5 or greater, in about 15, or about 30, or about 45, or about 60, or about 90 minutes. A modified-release composition that is unstable in near neutral or alkaline environments can release 70% or more by weight of the pharmaceutical composition (e.g., comprising a microbial cocktail) in the modified-release composition in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various aspects, the modified-release formulations can be designed for immediate release (e.g. upon ingestion). In various aspects, the modified-release compositions can have sustained-release profiles, i.e. slow release of the active ingredient(s) in the body (e.g., GI tract) over an extended period of time. In various aspects, the modified-release compositions can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various aspects, the modified-release compositions can utilize one or more modified-release coatings such as delayed-release coatings to provide for effective, delayed yet substantial delivery of the formulations to the GI tract together with, optionally, additional therapeutic agents.

In an aspect, the delayed-release coating includes an enteric agent that is substantially stable in acidic environments and substantially unstable in near neutral to alkaline environments. In an aspect, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In some aspects, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various aspects, the enteric agent can be a combination of the foregoing solutions or dispersions.

In certain aspects, one or more coating system additives are used with the enteric agent. For example, one or more PlasACRYLTM additives can be used as an anti-tacking agent coating additive. Illustrative PlasACRYLTM additives include, but are not limited to, PlasACRYLTM HTP20 and PlasACRYLTM T20.

In another aspect, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release compositions. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In an aspect, colonic delivery is achieved by use of a slowly eroding wax plug (e.g., various PEGS, including for example, PEG6000).

In a further aspect, the delayed-release coating can be degraded by a microbial enzyme present in the gut flora. In an aspect, the delayed-release coating can be degraded by bacteria present in the small intestine. In another aspect, the delayed-release coating can be degraded by bacteria present in the large intestine.

In various aspects, the modified release composition can be designed for release in the colon. Various colon-specific delivery approaches can be utilized. For example, the modified release composition can be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the composition can be designed as a core tablet with three layers of polymer. The first coating is an acid-soluble polymer (e.g., EUDRAGIT E), the outer coating is enteric, along with a hydroxypropyl methylcellulose barrier layer interposed in between. In another aspect, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an aspect, the composition is in the form of ionically crosslinked pectin beads which are further coated with a polymer (e.g., EUDRAGIT polymer). Additional colon specific compositions include, but are not limited to, pressure-controlled drug delivery systems (prepared with, for example, ethylcellulose) and osmotic controlled drug delivery systems (i.e., ORDS-CT).

Compositions for colon specific delivery of a pathogen antigen/fecal-derived adjuvant (and optionally additional therapeutic agents), as described herein, can be evaluated using, for example, in vitro dissolution tests. For example, parallel dissolution studies in different buffers can be undertaken to characterize the behavior of the compositions at different pH levels. Alternatively, in vitro enzymatic tests can be carried out. For example, in cases where the formulations comprise fecal bacteria, the compositions can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further aspect, in vivo evaluations can be carried out using animal models such as dogs, guinea pigs, rats, and pigs. Further, clinical evaluation of colon specific drug delivery compositions can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various aspects, the present composition provides for substantial uniform delivery of the formulations in the area of release in the GI tract. In an aspect, the present composition minimizes patchy or heterogeneous release of the formulation.

In various aspects, the present formulations provide for release of multiple doses of one or more bacterial mixtures along the GI tract. For example, the composition and/or formulation can release multiple doses of the same bacterial mixture at different locations along the intestines, at different times, and/or at different pH. Alternatively, the composition and/or formulation can release a dose of different bacterial mixtures at different locations along the intestines, at different times, and/or at a different pH. In an aspect, the pharmaceutical composition comprises a first bacterial mixture comprising one or more bacterial isolates that is released at a first location in the intestine, and a second bacterial mixture comprising a non-viable fecal bacterial preparation that is released at a second location in the intestine. In an aspect, the first bacterial mixture is released in the ileum, and the second bacterial mixture is released in the colon.

The overall release profile of such a formulation can be adjusted using, for example, multiple particle types or multiple layers. For example, in an aspect, a first bacterial mixture (or first dose of a bacterial mixture) can be formulated for release in, for example, the small intestine (e.g., one or more of duodenum, jejunum, ileum), whereas the second bacterial mixture (or second dose of the bacterial mixture) is formulated for delayed release in, for example, the large intestine (e.g., one or more of cecum, ascending, transverse, descending or sigmoid portions of the colon, and rectum). In another example, the first bacterial mixture (or first dose of a bacterial mixture) can be formulated for release in, for example, the small intestine (e.g., one or more of duodenum, jejunum, ileum), whereas the second bacterial mixture (or second dose of a bacterial mixture) is formulated for delayed release in, for example, another part of the small intestine (e.g., one or more of duodenum, jejunum, ileum). In another aspect, the first bacterial mixture (or first dose of a bacterial mixture) can be formulated for release in, for example, the large intestine (e.g., one or more of cecum, ascending, transverse, descending or sigmoid portions of the colon, and rectum), whereas the second bacterial mixture (or second dose of the bacterial mixture) is formulated for delayed release in, for example, another part of the large intestine (e.g., one or more of cecum, ascending, transverse, descending or sigmoid portions of the colon, and rectum). In various aspects, the composition and/or formulation can release at least one dose, at least two doses, at least three doses, at least four doses, or at least five doses of the bacterial mixture at different locations along the intestines, at different times, and/or at different pH. Likewise, in various aspects, the composition and/or formulation can release at least one bacterial mixture, at least two bacterial mixtures, at least three bacterial mixtures, at least four bacterial mixtures, or at least five bacterial mixtures at different locations along the intestines, at different times, and/or at different pH.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

It will be understood that a pharmaceutical composition described herein can comprise multiple distinct bacterial mixtures, for example to achieve different delivery location profiles for each bacterial mixture. In an aspect, a pharmaceutical composition comprises at least two bacterial mixtures, such that a first bacterial mixture comprises one or more bacterial isolates and a second bacterial mixture comprises a non-viable fecal bacterial preparation. In an aspect, the second bacterial mixture further comprises one or more bacterial isolates that are different than the bacterial isolates in the first bacterial mixture. Alternatively, the second bacterial mixture can consist essentially of the non-viable fecal bacterial preparation. In another aspect, the first bacterial mixture comprises only one bacterial isolate. A pharmaceutical composition can comprise any number of bacterial mixtures, for example one, two, three, four, five, six, seven, eight, nine, ten, or more than ten bacterial mixtures that each contain a different bacterial isolate, a different combination of bacterial isolates, a non-viable fecal bacterial preparation, or a different combination of a non-viable fecal bacterial preparation with one or more bacterial isolates.

In an aspect, a pharmaceutical composition can be a drench. In one aspect, a drench is prepared by choosing a saline-suspended form of a pharmaceutical composition. A watersoluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzenesulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a pharmaceutical composition can be administered by a patch.

In some aspects, the bacterial isolates described herein are in the form of live, vegetative cells. In some aspects, the bacterial isolates described herein are in the form of spores. In some aspects, the bacterial isolates described herewith are lyophilized. By way of non-limiting example, lyophilization can be via methods known in the art, including those described in U.S. Pat. No. 7,799,328, the contents of which are hereby incorporated by reference in their entirety. In some aspects, lyophilized bacterial mixtures described herein are placed in an enterically coated soft gel or capsule.

In various aspects, formulations can take the form of those described in one or more of U.S. Pat. Nos. 8,535,713 and 8,9117,77 and U.S. Pat. Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and U.S. Pat. Publication Nos. 2014/0302132; 2014/0227357; 20140088202; 20130287842; 2013/0295188; 2013/0307962; and 20130184290, the contents of which are hereby incorporated by reference in their entirety.

### Treatments, Administration and Dosage

In an aspect, the subject matter disclosed herein comprises a method of preventing an infection by a pathogen (e.g., viral or bacterial pathogen) in a subject by administering a pharmaceutical composition as described herein. In an aspect, the subject matter disclosed herein comprises a method of treating an infection caused by a pathogen in a subject by administering a pharmaceutical composition as described herein. In an aspect, disclosed herein is a method of preventing a disease or disorder caused by a viral infection. For example, the pathogen can be SARS-CoV-2 and the disease or disorder can be COVID-19.

In an aspect, the subject matter disclosed herein comprises a method of preventing or treating an viral infection in a subject by administering a pharmaceutical composition comprising fecal filtrate derived or separated from stool of a human donor. In another aspect, the human donor has a SARS-CoV-2 infection. In another aspect, the human donor recovered from a SARS-CoV-2 infection. In another aspect, the human donor recovered from SARS-CoV-2 within 1, 2, 3, 4, 5, 6, 7, 8, 9, 10, 11, or 12 months. In another aspect, the human donor who recovered from SARS-CoV-2, recovered from an asymptomatic SARS-CoV-2 infection. In another aspect, a human donor is a person who recovered from SARS-CoV-2 without the use of any antiviral treatment. In another aspect, a human donor is a person who recovered from SARS-CoV-2 with the use of any antiviral treatment. In yet another aspect, the human donor is actively shedding the SARS-CoV-2 virus. In a further aspect, the stool derived from a human donor comprises one or more viral antigen. In another aspect, the one or more viral antigen is or comprises a viral antigen for SARS-Co-V-2.

In an aspect, a formulation comprising one or more viral antigens and one or more fecal-derived adjuvants is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

Individual doses of the pharmaceutical composition can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, , about 2,000 mg, about 3,000 mg, about 4,000 mg, or about 5,000 mg of the active ingredient, inclusive of all values and ranges therebetween.

In an aspect, the pharmaceutical composition is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various aspects, the composition is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some aspects, a suitable dosage of the pharmaceutical composition is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other aspects, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain aspects, the pharmaceutical composition can be administered, for example, more than once daily, about once per day, about every other day, about every third day, about once a week, about once every two weeks, about once every month, about once every two months, about once every three months, about once every six months, or about once every year.

In an aspect, a pharmaceutical composition can be administered to a patient in need thereof at least once daily for at least two consecutive days. In another aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, or 12 consecutive weeks. In another aspect, a pharmaceutical composition is administered at least twice, three times, four times, or five times per week for at least 1, 2, 3, 4, 5, 6, 7, 8, 9, 10, 11, or 12 consecutive weeks. In another aspect, a pharmaceutical composition is administered at least once daily for at most 4, 5, 6, 7, 8, 9, 10, 11, 12, 13, 14, 15, 16, 17, 18, 19, or 20 consecutive days or weeks. In a further aspect, a pharmaceutical composition is administered at least once daily for at most 1, 2, 3, 4, 5, 6, 7, 8, 9, 10, 11, or 12 consecutive weeks or months. In yet another aspect, a pharmaceutical composition is administered at least once for at least 1, 2, 3, 4, 5, 6, 7, 8, 9, 10, 11, or 12 consecutive months or years, chronically for a subject’s entire life span, or an indefinite period of time.

In an aspect, a pharmaceutical composition can be administered to a patient in need thereof at least twice daily for at least two consecutive days. In an aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, or 12 consecutive weeks. In another aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, or 12 consecutive weeks or months. In another aspect, a pharmaceutical composition is administered at least twice for at least 1, 2, 3, 4, 5, 6, 7, 8, 9, 10, 11, or 12 consecutive months or years, chronically for a subject’s entire life span, or an indefinite period of time.

In an aspect of the present disclosure, a pharmaceutical composition can be administered to a patient in need thereof at least three times daily for at least two consecutive days. In an aspect, a pharmaceutical composition is administered at least three times daily for at least 3, 4, 5, 6, 7, 8, 9, 10, 11, 12, 13, 14, or 15 consecutive days. In an aspect, a pharmaceutical composition is administered at least three times daily for at least 1, 2, 3, 4, 5, 6, 7, 8, 9, 10, 11, or 12 consecutive weeks. In an aspect, a pharmaceutical composition is administered at least three times daily for at most 4, 5, 6, 7, 8, 9, 10, 11, 12, 13, 14, 15, 16, 17, 18, 19, or 20 consecutive days or weeks. In an aspect, a pharmaceutical composition is administered at least three times daily for at most 1, 2, 3, 4, 5, 6, 7, 8, 9, 10, 11, or 12 consecutive weeks or months. In an aspect, a pharmaceutical composition is administered at least three times for at least 1, 2, 3, 4, 5, 6, 7, 8, 9, 10, 11, or 12 consecutive months or years, chronically for a subject’s entire life span, or an indefinite period of time.

In an aspect, a pharmaceutical composition can be administered to a patient in need thereof at a dosing schedule of at least once or twice daily for at least three consecutive days or weeks. In an aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In an aspect, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In an aspect, a pharmaceutical composition can be administered to a patient in need thereof, wherein the administration comprises a first dosing schedule followed by a second dosing schedule. In an aspect, a first dosing schedule comprises an induction dose. In an aspect, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In an aspect, at least one of a first or second dosing schedule comprises administration of the composition at least once daily for at least 2, 3, 4, 5, 6, 7, 8, 9, 10, 11, 12, 13, 14, or 15 consecutive days. In an aspect, at least one of a first or second dosing schedule comprises administration of the composition at least once daily for at least 1, 2, 3, 4, 5, 6, 7, 8, 9, 10, 11, or 12 consecutive weeks. In an aspect, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In an aspect, at least one of a first or second dosing schedule comprises administration of the composition for at most 1, 2, 3, 4, 5, 6, 7, 8, 9, 10, 11, or 12 consecutive weeks or months. In an aspect, at least one of a first or second dosing schedule comprises administration of the composition for at least 1, 2, 3, 4, 5, 6, 7, 8, 9, 10, 11, or 12 consecutive months or years, chronically for a subject’s entire life span, or an indefinite period of time.

In an aspect, at least one of a first or second dosing schedule used in a method can be once-a-week, twice-a-week, or thrice-a-week.

In an aspect, at least one of a first and second dosing schedule can last for at least about 2, 4, 6, 8, 10, 12, 18, 24, 36, 48, 72, or 96 months. In an aspect, at least one of a first and second dosing schedule is an intermittent dosing schedule. In an aspect, at least one of a first and second dosing schedule is an intermittent dosing schedule comprising a period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In an aspect, at least one of a first and second dosing schedule comprises administering a dose every other day, every two days, or every 3, 4, 5, 6, 7, 8 days. In an aspect, a dose is administered for an extended period of time with or without titration (or otherwise changing the dosage or dosing schedule).

In an aspect, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In an aspect, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In another aspect, a second dosing schedule (e.g., a maintenance dosing schedule) has an equal or lower dosing frequency than a first dosing schedule. In an aspect, a second dosing schedule (e.g., a maintenance dosing schedule) has a higher dosing interval than a first dosing schedule.

In an aspect, a first dosing schedule comprises administration of a single dose of a pharmaceutical composition to a subject. In an aspect, a second dosing schedule comprises administration of either a single dose or multiple doses of the pharmaceutical composition to the subject, wherein the dose of the pharmaceutical composition during the second dosing schedule is less than the dose of the pharmaceutical composition during the first dosing schedule.

In various aspects, methods described herein are useful in vaccinating a human subject. In some aspects, the human is a pediatric human. In other aspects, the human is an adult human. In other aspects, the human is a geriatric human. In other aspects, the human may be referred to as a patient. In some aspects, the human is a female. In some aspects, the human is a male.

In certain aspects, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being vaccinated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1-year-old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

It will be appreciated that compositions, dosage forms, and medicaments as described herein include combination pharmaceutical compositions in which one or more additional compounds or medications are added to or otherwise co-administered with a purified fecal microbiota composition.

The disclosure also includes the following embodiments:

Embodiment 1. A pharmaceutical composition comprising a fecal filtrate and a viral peptide or portion thereof, wherein the viral peptide or portion thereof is capable of inducing an immune response in a subject administered the composition.

Embodiment 2. The pharmaceutical composition of embodiment 1, wherein the fecal filtrate is from a stool of a human donor.

Embodiment 3. The pharmaceutical composition of embodiment 2, wherein the stool comprises the viral protein or portion thereof.

Embodiment 4. The pharmaceutical composition of any one of embodiments 1-3, wherein the pharmaceutical composition comprises an intact viral particle comprising the viral peptide or portion thereof.

Embodiment 5. The pharmaceutical composition of any one of embodiments 1-4, wherein the viral peptide or portion thereof is denatured.

Embodiment 6. The pharmaceutical composition of embodiment 5, wherein the viral peptide or portion thereof is heat denatured.

Embodiment 7. The pharmaceutical composition of any one of embodiments 1-6, wherein the viral peptide or portion thereof is from a DNA virus.

Embodiment 8. The pharmaceutical composition of embodiment 7, wherein the DNA virus is selected from the group consisting of: a dsDNA virus, a ssDNA virus, and a dsDNA-RT virus.

Embodiment 9. The pharmaceutical composition of any one of embodiments 1-6, wherein the viral peptide or portion thereof is from an RNA virus.

Embodiment 10. The pharmaceutical composition of embodiment 9, wherein the RNA virus is selected from the group consisting of: a dsRNA virus, a (+) ssRNA virus, a (-) ssRNA virus, and an ssRNA-RT virus.

Embodiment 11. The pharmaceutical composition of embodiment 10, wherein the RNA virus is a (+) ssRNA virus.

Embodiment 12. The pharmaceutical composition of embodiment 11, wherein the (+) ssRNA virus is selected from the group consisting of Hepacivirus C, West Nile virus, dengue virus, and a coronavirus.

Embodiment 13. The pharmaceutical composition of embodiment 12, wherein the (+) ssRNA virus is a coronavirus, and the coronavirus is selected from the group consisting of a severe acute respiratory syndrome coronavirus (SARS-CoV), a severe acute respiratory syndrome coronavirus 2 (SARS-CoV-2) and a Middle East respiratory syndrome-related coronavirus (MERS-CoV).

Embodiment 14. The pharmaceutical composition of embodiment 13, wherein the coronavirus is SARS-CoV-2.

Embodiment 15. The pharmaceutical composition off embodiment 14, wherein the viral peptide or portion thereof comprises a Spike (S) protein, Envelope (E) protein, Membrane (M) protein or nucleocapsid (N) protein of the SARS-CoV-2.

Embodiment 16. The pharmaceutical composition of any one of embodiments 1-14, wherein the composition is formulated for oral delivery.

Embodiment 17. The pharmaceutical composition of embodiment 15, wherein the composition comprises a capsule.

Embodiment 18. The pharmaceutical composition of any one of embodiments 1-17, wherein the viral peptide or portion thereof is delivered to the intestine of the subject.

Embodiment 19. The pharmaceutical composition of embodiment 18, wherein the intestine is the small intestine.

Embodiment 20. The pharmaceutical composition of embodiment 18, wherein the intestine is the large intestine.

Embodiment 21. The pharmaceutical composition of any one of embodiments 18-20, wherein the viral peptide or portion thereof binds to the surface of an intestinal cell of the subject to produce the immune response.

Embodiment 22. The pharmaceutical composition of embodiment 20, wherein the intestinal cell is an epithelial cell.

Embodiment 23. The pharmaceutical composition of embodiment 21 or embodiment 22, wherein the viral peptide or portion thereof binds to a receptor expressed by the intestinal cell.

Embodiment 24. The pharmaceutical composition of embodiment 23, wherein the receptor comprises ACE2.

Embodiment 25. The pharmaceutical composition of any one of embodiments 1-24, wherein the immune response comprises production of an immunoglobulin antibody specific for the viral peptide or portion thereof.

Embodiment 26. The pharmaceutical composition of embodiment 25, wherein the immunoglobulin antibody is an IgG antibody or an IgA antibody.

Embodiment 27. The pharmaceutical composition of any one of embodiments 1-26, wherein the immune response is a humoral immune response.

Embodiment 28. The pharmaceutical composition of any one of embodiments 2-27, wherein the fecal filtrate comprises an adjuvant that potentiates the immune response.

Embodiment 29. The pharmaceutical composition of embodiment 28, wherein the adjuvant is derived from a bacterial cell of the stool of the human donor.

Embodiment 30. The pharmaceutical composition of embodiment 29, wherein the adjuvant comprises at least one of a peptide, a lipopolysaccharide, flagellin, a peptidoglycan, a lipoteichoic acid, a lipoprotein, or a membrane vesicle.

Embodiment 31. The pharmaceutical composition of embodiment 30, wherein the adjuvant comprises the lipopolysaccharide.

Embodiment 32. The pharmaceutical composition of embodiment 2, wherein the fecal filtrate comprises viable bacterial cells of the stool.

Embodiment 33. The pharmaceutical composition of embodiment 2, wherein the fecal filtrate does not comprise viable bacterial cells of the stool.

Embodiment 34. The pharmaceutical composition of any one of embodiments 1-33, wherein the fecal filtrate is lyophilized.

Embodiment 35. A method of vaccinating the subject comprising administering the pharmaceutical composition of any one of embodiments 1-34 to the subject.

Embodiment 36. The method of embodiment 35, wherein the subject is a healthy subj ect.

Embodiment 37. A pharmaceutical composition comprising (i) filtered and homogenized stool from a human donor, and (ii) a peptide or portion thereof from a coronavirus.

Embodiment 38. A pharmaceutical composition comprising (i) a fecal filtrate from a stool of a human donor and (ii) a peptide or portion thereof from a coronavirus.

Embodiment 39. The pharmaceutical composition of embodiment 37 or embodiment 38, wherein the coronavirus is SARS-CoV-2.

Embodiment 40. A method of vaccinating a subject in need thereof, the method comprising administering to the subject (i) a fecal filtrate; and (ii) a viral peptide or portion thereof; wherein the viral peptide or portion thereof induces an immune response in the subject.

Embodiment 41. The method of embodiment 40, wherein the fecal filtrate and the viral peptide or portion thereof are administered to the subject in different compositions.

Embodiment 42. The method of embodiment 40, wherein a pharmaceutical composition comprises the fecal filtrate and the viral peptide or portion thereof.

Embodiment 43. The method of embodiment 42, wherein the administering comprises administering the composition to the subject in multiple doses.

Embodiment 44. The method of embodiment 43, wherein the multiple doses comprise administration at least twice daily.

Embodiment 45. The method of embodiment 43 or embodiment 44, wherein the multiple doses comprise administration at least three times daily.

Embodiment 46. The method of any one of embodiments 44-45, wherein the multiple doses comprise administration at least once daily for at least two days.

Embodiment 47. The method of any one of embodiments 44-46, wherein the multiple doses comprise administration at least once daily for at least three days.

Embodiment 48. The method of any one of embodiments 44-47, wherein the multiple doses comprise administration at least once daily for at least 7 days.

Embodiment 49. The method of any one of embodiments 44-48, wherein the multiple doses comprise administration at least once daily for at least 10 days.

Embodiment 50. The method of any one of embodiments 44-49 wherein the multiple doses comprise administration at least once daily for at least 2 weeks.

Embodiment 51. The method of any one of embodiments 40-50, wherein the subject is a healthy human.

Embodiment 52. The method of any one of embodiments 40-51, wherein the viral peptide or portion thereof is from a coronavirus.

Embodiment 53. The method of embodiment 52, wherein the coronavirus is SARS-CoV-2.

Embodiment 54. A method of manufacturing a vaccine, the method comprising: incorporating into a pharmaceutical composition a fecal filtrate derived from a stool of a human donor, wherein the stool comprises a virus capable of infecting humans.

Embodiment 55. The method of embodiment 54, further comprising heating the virus to inactivate the virus.

Embodiment 56. The method of embodiment 55, wherein the virus is heated to a temperature of at least 40° C. for at least five hours.

Embodiment 57. The method of embodiment 55, wherein the virus is heated to a temperature of at least 55° C. for at least five hours.

Embodiment 58. The method of embodiment 55, wherein the virus is heated to a temperature of at least 60° C. for at least eight hours.

Embodiment 59. The method of any one of embodiments 55-58, wherein the virus is heated by heating the stool.

Embodiment 60. The method of any one of embodiments 55-58, wherein the virus is heated by heating the fecal filtrate comprising the virus.

Embodiment 61. The method of any one of embodiments 54-60, further comprising filtering the stool to produce the fecal filtrate.

Embodiment 62. The method of embodiment 61, wherein the filtering uses a filter having a pore size of no more than 300 µm.

Embodiment 63. The method of embodiment 62, wherein the filtering uses a filter having a pore size of no more than 200 µm.

Embodiment 64. The method of any one of embodiments 54-63, wherein the fecal filtrate comprises a peptide or portion thereof of the virus.

Embodiment 65. The method of embodiment 64, wherein the peptide or portion thereof comprises an epitope capable of inducing an immune response when administered to a human.

Embodiment 66. The method of embodiment 54-65, wherein the stool comprises at least 105 virions per gram of the stool.

Embodiment 67. The method of embodiment 66, wherein the stool comprises at least 106 virions per gram of the stool.

Embodiment 68. The method of embodiment 67, wherein the stool comprises at least 107 virions per gram of the stool.

Embodiment 69. The method of any one of embodiments 54-68, further comprising lyophilizing the fecal filtrate.

Embodiment 70. The method of embodiment 69, further comprising encapsulating the lyophilized fecal filtrate to produce the pharmaceutical composition.

Embodiment 71. The method of any one of embodiments 54-70, wherein the virus is a coronavirus.

Embodiment 72. The method of embodiment 71, wherein the coronavirus is SARS-CoV-2

Embodiment 73. A method, comprising obtaining a stool of a human donor, wherein the stool comprises a virus infectious to humans; heating the stool to inactivate the virus; following the heating, filtering the stool to produce a fecal filtrate; lyophilizing the fecal filtrate; and encapsulating the lyophilized fecal filtrate to produce a vaccine.

Embodiment 74. The method of embodiment 73, wherein the virus is a coronavirus.

Embodiment 75. The method of embodiment 71, wherein the coronavirus is SARS-CoV-2.

Embodiment 76. A method of inducing an immune response in a subject comprising administering to the subject a fecal filtrate comprising a viral peptide or portion thereof, wherein the fecal filtrate is derived from a stool of a human donor.

Embodiment 77. The method of embodiment 76, wherein the viral peptide or portion thereof is from a virus that is infectious to humans.

Embodiment 78. The method of embodiment 76 or embodiment 77, wherein the fecal filtrate is delivered to the intestine of the subject.

Embodiment 79. The method of any one of embodiments 76-78, wherein of viral peptide or portion thereof binds to the surface of an intestinal cell of the subject to produce the immune response.

Embodiment 80. The method of embodiment 79, wherein the intestinal cell is an epithelial cell.

Embodiment 81. The method of embodiment 79 or embodiment 80, wherein the viral peptide or portion thereof binds to a receptor expressed by the intestinal cell.

Embodiment 82. The method of embodiment 81, wherein the receptor comprises ACE2.

Embodiment 83. The method of any one of embodiments 76-82, wherein the immune response comprises production of an immunoglobulin antibody that recognizes the viral peptide or portion thereof.

Embodiment 84. The method of embodiment 83, wherein the immunoglobulin antibody is an IgG antibody or an IgA antibody.

Embodiment 85. The method of any one of embodiments 76-84, wherein the immune response is a humoral immune response.

Embodiment 86. The method of any one of embodiments 76-85, wherein the fecal filtrate comprises an adjuvant that potentiates the immune response.

Embodiment 87. The method of embodiment 86, wherein the adjuvant is derived from a bacterial cell of the stool of the human donor.

Embodiment 88. The method of embodiment 87, wherein the adjuvant comprises at least one of a peptide, a lipopolysaccharide, flagellin, a peptidoglycan, a lipoteichoic acid, a lipoprotein, or a membrane vesicle.

Embodiment 89. The method of embodiment 88, wherein the adjuvant comprises the lipopolysaccharide.

Embodiment 90. The method of any one of embodiments 40-53, wherein the fecal filtrate is derived from a stool of a human donor.

Embodiment 91. The method of embodiment 90, wherein the stool comprises the viral peptide or portion thereof.

Embodiment 92. The method of embodiment 91, wherein the stool does not comprise the viral peptide or portion thereof.

Embodiment 93. The method of embodiment 92, wherein the viral peptide or portion thereof is from a cultured virus.

Embodiment 94. A pharmaceutical composition comprising filtered and homogenized stool and a viral peptide or portion thereof, wherein the viral peptide or portion thereof is capable of inducing an immune response in a subject administered the composition.

Embodiment 95. A pharmaceutical composition comprising a fecal filtrate from a donor recovered from a viral infection.

Embodiment 96. The pharmaceutical composition of embodiment 95, where the fecal filtrate is a non-cellular fecal filtrate.

Embodiment 97. The pharmaceutical composition of embodiment 95 or 96, where the viral infection is a SARS-CoV-2 viral infection.

Embodiment 98. The pharmaceutical composition of any of embodiments 95 to 97, wherein the fecal filtrate comprises a viral peptide or portion thereof.

Embodiment 99. The pharmaceutical composition of embodiment 98, wherein the viral peptide or portion thereof is antigenic.

Embodiment 100. The pharmaceutical composition of embodiment 98, where the pharmaceutical composition is a vaccine.

Embodiment 101. The pharmaceutical composition of any of embodiments 95 to 100, wherein the fecal filtrate is frozen or lyophilized.

Embodiment 102. The pharmaceutical composition of any of embodiments 95 to 101, where the fecal filtrate further comprises bacteriophages.

Embodiment 103. The pharmaceutical composition of any of embodiments 95 to 102, wherein the fecal filtrate is capable of passing through a 0.2 micron filter.

Embodiment 104. The pharmaceutical composition of any of embodiments 95 to 103, wherein the fecal filtrate is a cellular fecal filtrate.

Embodiment 105. The pharmaceutical composition of any of embodiments 95 to 104, wherein the pharmaceutical composition is in an enema form.

Embodiment 106. The pharmaceutical composition of any of embodiments 95 to 105, wherein the pharmaceutical composition comprises fecal filtrates from multiple donors.

Embodiment 107. A method for treating or preventing one or more conditions in a subject in need thereof, the method comprising administering to the subject an amount of a pharmaceutical composition comprising a donor-derived fecal filtrate, wherein the donor has or recovered from a viral infection.

Embodiment 108. The method of embodiment 107, wherein the viral infection is a SARS-CoV-2 viral infection.

Embodiment 109. The method of embodiment 107, wherein the one or more conditions is SARS-CoV-2.

Embodiment 110. The method of any of embodiments 107 to 14, wherein the fecal filtrate is a non-cellular fecal filtrate.

Embodiment 111. The method of any of embodiments 107 to 110, wherein the fecal filtrate comprises a viral peptide or portion thereof.

Embodiment 112. The method of any of embodiments 107 to 111, wherein the fecal filtrate comprises a viral peptide or portion thereof.

Embodiment 113. The method of embodiment 112, wherein the viral peptide or portion thereof is antigenic.

Embodiment 114. The method of any of embodiments 107 to 113, wherein the pharmaceutical composition is a vaccine.

Embodiment 115. The method of any of embodiments 107 to 114, wherein the fecal filtrate is frozen or lyophilized.

Embodiment 116. The method of any of embodiments 107 to 115, wherein the fecal filtrate further comprises bacteriophages.

Embodiment 117. The method of any of embodiments 107 to 116, wherein the fecal filtrate is capable of passing through a 0.2 micron filter.

Embodiment 118. The method of any of embodiments 107 to 117, wherein the administering is by administering a capsule comprising the pharmaceutical composition.

Embodiment 119. The method of any of embodiments 107 to 118, wherein the administering is a single dose.

Embodiment 120. The method of any of embodiments 107 to 118, wherein the administering comprises multiple doses.

Embodiment 121. The method of any of embodiments 107 to 120, wherein the amount is a therapeutic amount sufficient to produce an immune response against an active SARS-CoV-2 virus.

Embodiment 122. A vaccine comprising filtered and homogenized stool material from a human donor infected with the SARS-CoV-2 virus, wherein the stool material comprises a peptide or portion thereof from the SARS-CoV-2 virus in an amount sufficient to produce an immune response against an active SARS-CoV-2 virus in a subject, and wherein the stool material is pasteurized.

Embodiment 123. The vaccine of embodiment 122, wherein the stool material is a non-cellular stool material.

Embodiment 124. The vaccine of any of embodiments 122 to 123, wherein the stool material is capable of passing through a 0.2 micron filter.

Embodiment 125. A method of preventing a SARS-CoV-2 virus infection in a subject, the method comprising administering to the subject a vaccine comprising filtered and homogenized stool material from a human donor infected with SARS-CoV-2 virus, wherein the stool material comprises a peptide or portion thereof from the SARS-CoV-2 virus in an amount sufficient to produce an immune response against an active SARS-CoV-2 virus.

Embodiment 126. A method of producing a vaccine against the SARS-CoV-2 virus, the method comprising: selecting stool of a donor infected with SARS-CoV-2 virus, and filtering and homogenizing the stool to generate stool material comprising a peptide or portion thereof from the SARS-CoV-2 virus.

Embodiment 127. A method comprising: obtaining a stool of a human donor, wherein the stool comprises an infectious virus, and wherein the stool comprises fecal material; pasteurizing the stool at a temperature to inactivate the virus; filtering and homogenizing the pasteurized stool to produce a stool composition; testing the fecal material for the presence of one or more viral antigens comprising incubating the fecal material in a testing apparatus comprising an anti-SARS-CoV-2 antibody; and formulating the stool composition as a pharmaceutical composition on the basis of the presence of one or more viral antigens in the fecal material.

Embodiment 128. The method of embodiment 127, wherein the pasteurizing is for 1 to 10 hrs.

Embodiment 129. The method of embodiment 127, wherein the pasteurizing is for up to 10 hrs.

Embodiment 130. The method of embodiment 127, wherein the pasteurizing is for at least 5 hrs.

Embodiment 131. The method of any one of embodiments 127 - 130, wherein the temperature is at between 50 and 140° C.

Embodiment 132. The method of embodiment 127 - 130, wherein the temperature is between 50 and 90° C.

Embodiment 133. The method of embodiment 127 - 130, wherein the temperature is 60° C.

Embodiment 134. The method of embodiment 127, further comprising washing the testing apparatus with a wash buffer.

Embodiment 135. The method of embodiment 127, wherein the apparatus comprises a stabilizer selected from the group consisting of sucrose, glycine, potassium acetate, and sorbitol.

Embodiment 136. The method of embodiment 127, wherein the testing comprises self-sandwich ELISA.

Embodiment 137. The method of embodiment 136, where the anti-SARS-CoV-2 antibody is a polyclonal antibody.

Embodiment 138. The method of embodiment 127, wherein the anti-SARS-CoV-2 antibody is fixed to a surface of the apparatus.

Embodiment 139. The method of embodiment 138, wherein the anti-SARS-CoV-2 antibody binds to the S (spike) protein of SARS-CoV-2.

Embodiment 140. The method of embodiment 127, wherein the anti-SARS-CoV-2 antibody is detected by a second antibody selected from the group consisting of an anti-Histidine (anti-His) tag antibody, an HRP conjugated antibody, an HRP-Streptavidin conjugated antibody, or a biotin conjugated antibody.

Embodiment 141. The method of embodiment 127, wherein the filtering comprises passing the pasteurized stool through a filter having a pore size of no greater than 0.2 µm.

Embodiment 142. The method of embodiment 127, wherein the apparatus further comprises testing solutions comprising a blocking buffer, a wash buffer, a substrate, and stop solution.

Embodiment 143. A method of testing comprising: obtaining a stool of a human donor, wherein the stool comprises an infectious virus, and wherein the stool comprises fecal material; inactivating the virus to produce a treated stool sample; testing the fecal material for the presence of one or more viral antigens comprising incubating the fecal material in a testing apparatus comprising an anti-SARS-CoV-2 primary antibody; filtering the treated stool sample to produce a fecal filtrate comprising the inactivated virus or portion thereof; and formulating the fecal filtrate as a vaccine on the basis of the presence of one or more viral antigens in the fecal material.

Embodiment 144. The method of embodiment 143, wherein the inactivating is through a treatment selected from the group consisting of pasteurization, terminal dry heat, vapour heat, solvent or detergent, precipitation, chromatography, and nanofiltration.

Embodiment 145. A method comprising: obtaining a stool of a human donor, wherein the stool comprises an infectious virus; inactivating the virus to produce a treated stool sample; testing for the presence of viral antigens comprising incubating fecal material from the stool sample in a testing apparatus comprising a tagged antibody fixed to a surface of the apparatus and a human ACE2 receptor antibody linked by the tag to the tagged antibody; and formulating the treated stool sample as a vaccine based on detecting the presence of viral antigens in the stool sample.

Embodiment 146. The method of embodiment 145, wherein the inactivating is through a treatment selected from the group consisting of pasteurization, terminal dry heat, vapour heat, solvent or detergent, precipitation, chromatography, and nanofiltration.

Embodiment 147. The method of embodiment 145, wherein the stool sample is diluted prior to the testing.

Embodiment 148. The method of embodiment 145, wherein the S protein a SARS-CoV-2 virus binds to the ACE2 receptor antibody during the incubation.

Embodiment 149. The method of embodiment 148, further comprising detecting the binding of the SARS-CoV-2 virus to the ACE2 receptor.

Embodiment 150. The methods of embodiments 143 or 145, further comprising filtering the treated stool sample to produce a fecal filtrate; lyophilizing the fecal filtrate; and encapsulating the lyophilized fecal filtrate to produce a vaccine.

Embodiment 151. A method for preparing a vaccine comprising: obtaining a stool of a human donor, wherein the stool comprises an infectious virus; inactivating the infectious virus to produce a stool sample; filtering and homogenizing the stool sample to produce a fecal filtrate comprising fecal material; testing the fecal material for the presence of viral antigens comprising: a) incubating the fecal material in a testing apparatus comprising an anti-SARS-CoV-2 antibody; or b) incubating the fecal material in a testing apparatus comprising a tagged antibody fixed to a surface of the apparatus and a human ACE2 receptor antibody linked by the tag to the tagged antibody; lyophilizing the fecal material; and/or encapsulating the lyophilized fecal material to produce a pharmaceutical composition; wherein the lyophilizing and/or the encapsulating are based on detecting the presence of viral antigens in the fecal material.

Embodiment 152. A method for diagnosing an infectious virus in a subject comprising: obtaining a stool of a human donor, wherein the stool comprises an infectious virus; filtering and homogenizing the stool to produce a fecal filtrate comprising fecal material; and testing for the presence of the virus in the fecal material comprising: a) incubating the fecal material in a testing apparatus comprising an anti-SARS-CoV-2 antibody; or b) incubating the fecal material in a testing apparatus comprising a tagged antibody fixed to a surface of the apparatus and a human ACE2 receptor antibody linked by the tag to the tagged antibody; and formulating the fecal filtrate as a pharmaceutical composition based on detecting the presence of the virus in the fecal material.

Embodiment 153. The method of embodiment 152, further comprising pasteurizing the stool prior to filtering to inactivate the infectious virus.

Embodiment 154. A vaccine comprising a pasteurized fecal filtrate from a donor having or recovered from a viral infection.

Embodiment 155. The vaccine of embodiment 154, where the fecal filtrate is a non-cellular fecal filtrate.

Embodiment 156. The vaccine of embodiment 154 or 155, where the infectious virus is SARS-CoV-2.

Embodiment 157. The vaccine of any of embodiments 154 to 156, wherein the fecal material comprises a viral peptide or portion thereof.

Embodiment 158. The vaccine of embodiment 157, wherein the viral peptide or portion thereof is antigenic.

Embodiment 159. The vaccine of embodiment 154, where the pharmaceutical composition is a vaccine.

Embodiment 160. The vaccine of any of embodiments 154 to 159, wherein the fecal filtrate is frozen or lyophilized.

Embodiment 161. The vaccine of any of embodiments 154 to 160, wherein the fecal filtrate is capable of passing through a 0.2 micron filter.

Embodiment 162. The vaccine of any of embodiments 154 to 160, wherein the fecal filtrate is a cellular fecal filtrate.

Embodiment 163. The vaccine of any of embodiments 152 to 162, wherein the pharmaceutical composition is in an enema form.

Embodiment 164. The vaccine of any of embodiments 154 to 163, wherein the pharmaceutical composition comprises fecal filtrates from multiple donors.

Embodiment 165. A method of detecting SARS-CoV-2 infection in a subject in need thereof, comprising: obtaining a fecal filtrate, and subjecting the fecal filtrate to an immunoassay.

Embodiment 166. A method of selecting a fecal donor for developing a SARS-CoV-2 vaccine comprising: determining if the donor has or recovered from a SARS-Co-V2 viral infection comprising the method of embodiment 150.

Embodiment 167. A method comprising: selecting a donor based on a presence of a pathogen in a stool of the donor; extracting fecal material from the stool; and formulating the fecal material as a vaccine for administration to a subject.

Embodiment 168. The method of embodiment 167, wherein the pathogen is a bacterial pathogen.

Embodiment 169. The method of embodiment 168, wherein the bacterial pathogen is selected from the group consisting of: Mycobacterium sp., Mycobacterium tuberculosis, Mycobacterium bovis (e.g., Mycobacterium bovis BCG), Corynebacterium sp., Corynebacterium diphtheriae, Clostridium sp., Clostridium tetani, Bordetella sp., Bordetella pertussis, Haemophilus sp., Haemophilus influenzae (e.g., type b), Vibrio sp., Vibrio cholerae, Salmonella sp., Salmonella enterica (e.g., Salmonella enterica subsp. enterica), Salmonella typhimurium, Salmonella typhi, Streptococcus sp., Streptococcus pneumoniae, Fusobacterium sp., Fusobacterium nucleatum, Listeria sp., Listeria monocytogenes, Listeria ivanovii, Listeria grayi, Yersinia sp., Yersinia pestis, enterocolitica, Yersinia pseudotuberculosis, Shigella sp., Shigella dysenteriae, Shigella flexneri, Shigella boydii, Shigella sonnei, Neisseria sp., Neisseria meningitidis, Bacillus sp., Bacillus anthracis, Francisella tularensis, Rickettsia prowazekii, Coxiella burnetiid, and a combination thereof.

Embodiment 170. The method of embodiment 167, wherein the pathogen is a virus.

Embodiment 171. The method of embodiment 170, wherein the virus is selected from the group consisting of: selected from the group consisting of Dengue virus, Hepatitis A virus, a Hepadnavirus, Hepatitis B virus, Hepatitis C virus, Hepatitis D virus (HDV), Hepatitis E virus, Human papillomavirus, an Orthomyxovirus, Influenza virus (e.g., Influenza virus A, Influenza virus B, Influenza virus C, or Influenza virus D), Japanese encephalitis virus, Polio virus, Rotavirus, Rubella virus, Variola virus, Adenovirus, Coxsackie B virus, Enterovirus 71, Human immunodeficiency virus, Human T-cell lymphotropic virus (HTLV-I), Norovirus, Respiratory syncytial virus, Poxvirus, Vaccinia virus, Paravaccinia virus, Cowpox virus, Monkeypox virus, Bovine popular stomatitis virus, Orf virus, Smallpox virus, Herpesvirus, Herpes simplex virus (HSV-1 or HSV-2), Varicella zoster virus (VZV or HHV-3), Epstein-Barr virus (EBV or HHV-4), Cytomegalovirus (HCMV or HHV-5), Human herpesvirus 6A (HHV-6A), Human herpesvirus 6B (HHV-6V), Human herpesvirus 7 (HHV-7), Kaposi’s sarcoma-associated herpesvirus (KSHV or HHV-8), Tanapox virus, Yaba monkey tumor virus, Molluscum contagiosum virus, a Flavivirus, West Nile virus, Tick-bome encephalitis virus, Yellow fever virus, Zika virus, Palm Creek virus (PCV), Parramatta River virus (PaRV), an Alphavirus, the Barmah Forest virus, Chikungunya virus, Mayaro virus, O′nyong′nyong virus, Ross River virus, Semliki Forest virus, Sindbis virus, Una virus, Eastern Equine encephalitis virus, Tonate virus, Venezuelan equine encephalitis virus, Western equine encephalitis virus, an Asfaviridae, Togavirus, Paramyxovirus, Measles virus, Mumps virus, Rhabdovirus, Rabies virus, Bunyavirus, Crimean-Congo hemorrhagic fever orthonairovirus (CCHFV), Hantavirus, Puumala orthohantavirus (PUUV), a Filovirus, Ebola virus, Marburg virus, a coronavirus, SARS-CoV, a severe acute respiratory syndrome coronavirus 2 (SARS-CoV-2) and a Middle East respiratory syndrome-related coronavirus (MERS-CoV). and a combination thereof.

Embodiment 172. The method of any one of embodiments 167-171, further comprising testing the stool of the donor for the pathogen.

Embodiment 173. The method of any one of embodiments 167-172, further comprising pasteurizing the stool to inactivate the pathogen.

Embodiment 174. The method of any one of embodiments 167-173, wherein extracting the fecal material comprises homogenizing and filtering the stool to produce a fecal filtrate comprising the fecal material.

Embodiment 175. The method of embodiment 174, wherein formulating the fecal material as a vaccine comprises lyophilizing the fecal filtrate.

Embodiment 176. The method of embodiment 175, further comprising encapsulating the lyophilized fecal filtrate.

Embodiment 177. The method of embodiment 171, wherein the virus is SARS-CoV-2.

Embodiment 178. A method of manufacturing a vaccine comprising: selecting a stool of a donor based on a presence of SARS-CoV-2 in the stool; inactivating the SARS-CoV-2 by pasteurizing the stool; homogenizing and filtering the pasteurized stool to produce a fecal filtrate; lyophilizing the fecal filtrate; and encapsulating the lyophilized fecal filtrate to produce the vaccine.

Embodiment 179. The method of embodiment 178, further comprising detecting the presence of the SARS-CoV-2 in fecal material from the stool.

Embodiment 180. The method of embodiment 179, wherein the detecting comprises an ELISA assay.

Embodiment 181. The method of embodiment 180, wherein the ELISA assay is a sandwich ELISA assay.

Embodiment 182. The method of embodiment 180, wherein the ELISA assay is a self-sandwich ELISA assay.

Embodiment 183. The method of any one of embodiments 179 to 182, wherein the ELISA assay comprises detecting the S protein of the SARS-CoV-2 in the fecal material.

Embodiment 184. A pharmaceutical composition comprising a fecal bacterial preparation, wherein the fecal bacterial preparation comprises fecal bacteria derived from a stool of a human, and wherein the fecal bacteria are not viable.

Embodiment 185. The pharmaceutical composition of embodiment 184, wherein the fecal bacterial preparation comprises non-bacterial microbes.

Embodiment 186. The pharmaceutical composition of embodiment 185, wherein the non-bacterial microbes are not viable.

Embodiment 187. The pharmaceutical composition of any one of embodiments 184 to 186, wherein the fecal bacterial preparation comprises ruptured bacterial cells and vesicles derived from the ruptured bacterial cells.

Embodiment 188. The pharmaceutical composition of any one of embodiments 184 to 187, wherein the fecal bacterial preparation is lyophilized or spray-dried.

Embodiment 189. A pharmaceutical composition comprising a microbial preparation comprising microbes derived from a stool of a human donor, wherein the microbes are not viable.

Embodiment 190. The pharmaceutical composition of embodiment 189, wherein the microbial preparation is lyophilized or spray-dried.

Embodiment 191. A pharmaceutical composition comprising a sterile fecal microbiota, wherein the fecal microbiota is extracted from a stool of a human donor.

Embodiment 192. The pharmaceutical composition of embodiment 191, wherein the sterile fecal microbiota is lyophilized.

Embodiment 193. The pharmaceutical composition of any one of embodiments 184 to 192, wherein the pharmaceutical composition is in the form of a capsule.

Embodiment 194. The pharmaceutical composition of embodiment 193, wherein the capsule comprises an enteric coating.

Embodiment 195. The pharmaceutical composition of embodiment 193, wherein the capsule comprises enteric microcapsules.

Embodiment 196. A method of treating a disorder, the method comprising administering to a subject in need thereof a pharmaceutical composition comprising a fecal bacterial preparation, wherein the fecal bacterial preparation does not contain viable bacteria.

Embodiment 197. The method of embodiment 196, further comprising administering an antibiotic to the subject prior to administering the fecal bacterial preparation.

Embodiment 198. The method of embodiment 196 or embodiment 197, wherein the disorder is selected from the group consisting of inflammatory bowel disease, ulcerative colitis, Crohn’s disease and diabetes.

Embodiment 199. The method of embodiment 198, wherein the disorder is diabetes and the diabetes is Type 1 diabetes or Type 2 diabetes.

Embodiment 200. The method of any one of embodiments 196 to 199, wherein the subject has or is at risk of developing small intestinal bacterial overgrowth (SIBO).

Embodiment 201. The method of any one of embodiments 196 to 200, wherein the pharmaceutical composition comprises a capsule.

Embodiment 202. The method of embodiment 201, wherein the fecal bacterial preparation is released from the capsule in the subject’s small intestine.

Embodiment 203. A method of treating a disorder in a subject in need thereof, the subject in need thereof having small intestinal bacterial overgrowth (SIBO), the method comprising administering to the subject a pharmaceutical composition comprising a sterile fecal microbiota.

Embodiment 204. The method of embodiment 203, wherein the fecal microbiota is extracted from the stool of a human.

Embodiment 205. The method of embodiment 203 or embodiment 204, wherein the disorder is selected from the group consisting of inflammatory bowel disease, ulcerative colitis, Crohn’s disease and diabetes.

Embodiment 206. A method comprising: receiving a stool or portion thereof from a human donor, wherein the stool comprises a fecal microbiota; sterilizing the fecal microbiota; and incorporating the sterilized fecal microbiota into a pharmaceutical composition.

Embodiment 207. The method of embodiment 206, wherein the method further comprises homogenizing the stool or portion thereof in a diluent to produce a homogenate.

Embodiment 208. The method of embodiment 207, wherein the homogenizing is performed prior to sterilizing the fecal microbiota.

Embodiment 209. The method of embodiment 207, wherein the homogenizing is performed after sterilizing the fecal microbiota.

Embodiment 210. The method of any one of embodiments 207 to 209 further comprising filtering the homogenate.

Embodiment 211. The method of any one of embodiments 206 to 210, further comprising lyophilizing the sterilized microbiota.

Embodiment 212. The method of embodiment 211, further comprising encapsulating the lyophilized sterilized microbiota.

Embodiment 213. The method of any one of embodiments 206 to 212, wherein the sterilizing comprises treating the fecal microbiota with at least one of heat, radiation, sonication or an antibiotic.

Embodiment 214. The method of embodiment 213, wherein treating the fecal microbiota with heat comprises autoclaving the fecal microbiota.

Embodiment 215. The method of embodiment 213, wherein treating the fecal microbiota with heat comprises pasteurizing the fecal microbiota.

Embodiment 216. The method of any one of embodiments 206 to 215, wherein the fecal microbiota comprises bacteria.

Embodiment 217. The method of embodiment 216, wherein the method further comprises treating the bacteria with a germinant prior to the sterilizing.

Embodiment 218. The method of embodiment 216 or embodiment 217, wherein the sterilized fecal microbiota comprises vesicles derived from the bacteria.

Embodiment 219. The method of embodiment 218, further comprising concentrating the vesicles.

Embodiment 220. The method of embodiment 219, wherein concentrating the vesicles comprises ultracentrifugation or filtration.

Embodiment 221. The method of any one of embodiments 218 to 220, wherein the sterilizing comprises sonication.

Embodiment 222. A method of stimulating the immune system in a subject in need thereof, the method comprising administering to the subject a pharmaceutical composition comprising an adjuvant comprising a fecal bacterial preparation, wherein the fecal bacterial preparation comprises fecal bacteria from a stool of a human donor.

Embodiment 223. The method of embodiment 222, wherein the fecal bacteria are not viable.

Embodiment 224. The method of embodiment 222 or embodiment 223, wherein the subject is further administered an immunomodulator.

Embodiment 225. The method of embodiment 224, wherein the immunomodulator stimulates the immune system of the subject.

Embodiment 226. The method of embodiment 224, wherein the immunomodulator comprises an antigen of a pathogen.

Embodiment 227. The method of embodiment 226, wherein the fecal bacterial preparation comprises the antigen of the pathogen.

Embodiment 228. The method of embodiment 226 or embodiment 227, wherein the pathogen is a virus.

Embodiment 229. The method of embodiment 226 or embodiment 227, wherein the pathogen is a bacterial pathogen.

Embodiment 230. The method of embodiment 224, wherein the immunomodulator is administered in a pharmaceutical composition that does not comprise the fecal bacterial preparation.

Embodiment 231. The method of embodiment 224, wherein the immunomodulator is an immunotherapy.

Embodiment 232. The method of embodiment 231, wherein the immunotherapy comprises an anti-cancer immunotherapy for treatment of a cancer.

Embodiment 233. The method of embodiment 232, wherein the anti-cancer immunotherapy is selected from the group consisting of dendritic cell-based pump-priming or vaccination, T-cell adoptive transfer checkpoint inhibitor therapy, and a combination thereof.

Embodiment 234. The method of embodiment 233, wherein the anti-cancer immunotherapy is checkpoint inhibitor therapy.

Embodiment 235. The method of embodiment 234, wherein the checkpoint inhibitor therapy comprises administering to the subject a compound that recognizes a checkpoint molecule selected from the group consisting of PD-1, PD-L1, PD-L2, ICOS, ICOSL, and CTLA-4.

Embodiment 236. The method of embodiment 234, wherein the immunotherapy comprises administering to the subject a compound selected from the group consisting of Pembrolizumab, Nivolumab, or Ipilimumab.

Embodiment 237. The method of any one of embodiments 232 to 236, wherein the cancer is lung cancer.

Embodiment 238. The method of embodiment 237, wherein the lung cancer is non-small cell lung cancer.

Embodiment 239. The method of embodiment 231, wherein the immunotherapy is an allergen immunotherapy.

Embodiment 240. The method of embodiment 239, wherein the allergen immunotherapy comprises administering an allergen to the subject.

Embodiment 241. The method of embodiment 240, wherein the allergen comprises a food allergen.

Embodiment 242. The method of embodiment 241, wherein the food allergen comprises a peanut allergen.

Embodiment 243. The method of embodiment 239, wherein the allergen immunotherapy is administered to treat a condition of the subject selected from the group consisting of allergic rhinitis, asthma, seasonal pollinosis, perennial allergy, an allergy and a combination thereof.

Embodiment 244. The method of embodiment 243, wherein the condition is an allergy selected from the group consisting of an allergy to peanuts, soybeans, cow’s milk, milk protein, tree nuts, shellfish, fish, wheat, eggs, sesame, and dairy; seasonal allergies; outdoor allergies including grass and trees; animal or pet allergies; insect allergies; and mold allergies and a combination thereof.

Embodiment 245. The method of any one of embodiments 222 to 244, wherein the pharmaceutical composition comprises a capsule.

Embodiment 246. The method of embodiment 222, wherein the uncultured fecal bacteria are released from the capsule in the subject’s small intestine.

Embodiment 247. A pharmaceutical composition for adjuvant therapy comprising a non-viable fecal bacterial preparation.

Embodiment 248. The pharmaceutical composition of embodiment 247, wherein the non-viable fecal bacterial preparation is lyophilized or spray-dried.

Embodiment 249. The pharmaceutical composition of embodiment 247 or embodiment 248, wherein the pharmaceutical composition is in the form of a capsule.

Embodiment 250. The pharmaceutical composition of embodiment 249, wherein the capsule comprises an enteric coating.

Embodiment 251. The pharmaceutical composition of embodiment 249, wherein the capsule delivers the preparation to the small intestine.

Embodiment 252. The pharmaceutical composition of embodiment 227, wherein the adjuvant therapy is used before, during, or after immunotherapy.

Embodiment 253. The pharmaceutical composition of embodiment 252, wherein the immunotherapy is selected from the group consisting of dendritic cell-based pump-priming or vaccination, T-cell adoptive transfer checkpoint inhibitor therapy, and a combination thereof.

Embodiment 254. A method for treating various gastrointestinal-related conditions and disorders with non-viable microbes derived or extracted from stool of a human donor.

Embodiment 255. A pharmaceutical composition comprising a non-viable fecal bacterial preparation, wherein the preparation comprises non-viable fecal bacterial cells or components thereof, and does not comprise viable bacterial cells.

Embodiment 256. A pharmaceutical composition comprising a preparation of non-viable microbes, wherein the microbes are derived from a stool of a human.

Embodiment 257. A method of treating a disorder, the method comprising administering to a subject in need thereof a pharmaceutical composition comprising a preparation of uncultured fecal bacteria, wherein the preparation of uncultured fecal bacteria does not contain viable bacteria.

Embodiment 258. A method of treating an allergy in a subject, the method comprising administering to a subject in need thereof a pharmaceutical composition comprising a preparation of uncultured fecal bacteria, wherein the preparation of uncultured fecal bacteria does not contain viable bacteria.

Embodiment 259. A pharmaceutical composition for adjuvant therapy comprising a preparation of non-viable, uncultured fecal bacteria.

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### EXAMPLES

### Example 1: Product Development

Many vaccine development efforts are slow to develop and/or scale-up due to manufacturing challenges. An existing manufacturing platform supports a pivotal program in C. difficile treatment and uses healthy donors as natural bioreactors to produce microbiota that can be harvested from stool. An approach to combat COVID-19 is developed to take advantage of high titers of SARS-CoV-2 epitopes shed in COVID-19 patient stool as a source of both antigen and adjuvant for an oral vaccine. The high burden of the COVID-19 disease is used as a scaling factor with COVID-19 patients serving as our bioreactors, which limits the need for complex manufacturing infrastructure.

A SARS-CoV-2 positive donor is screened and the raw donor stool is immediately pasteurized at 60° C. for ~10 hours, inactivating viral pathogens but retaining key structural features of SARS-CoV-2 proteins and bacterial cellular debris (e.g. LPS) that can serve as adjuvants. Pasteurized stool is then homogenized and filtered to at least 0.2 microns, eliminating bacteria. Filtrate is lyophilized, milled and encapsulated (e.g., in double-encapsulated Hypromellose capsules). Double-encapsulated Hypromellose capsules are delivered orally to a subject in need with small-bowel release.

Proteins expressed from SARS-CoV-2 (e.g., Spike, Membrane and Nucleoprotein) are predicted to include epitopes capable of eliciting an IgG response. Without being bound to any scientific theory, exposure to these epitopes in the small bowel, coupled with the adjuvant effect of bacterial cellular debris is expected to lead to production of anti-SARS-CoV-2 antibodies (e.g., IgG), providing humoral immunity against COVID-19. Further, without being bound to any scientific theory, lipopolysaccharide (LPS), flagellin, peptidoglycans and other bacterial cellular debris delivered to the small bowel serve as potent adjuvants, inducing an immune response and overcoming tolerance. Tolerance is evaluated in a clinical study. Trial subjects are evaluated for an anti-SARS-CoV-2 IgG response at 4 weeks. The resulting data is used to determine the availability for the first in-human clinical study.

Purity assays are performed to determine the sterility of the final drug substance. Pharmaceutical potency is assayed via ELISA with anti-SARS-CoV-2 antibodies to measure SARS-CoV-2 epitopes in final drug substance.

Pharmaceutical effects are assessed via measurement of anti-SARS-CoV-2 IgG in subjects at 2 and 4 weeks after first administration.

Some advantages of this therapeutic product include speed, scale, and route of administration. Based on existing capabilities and experience, it is estimated that the time to clinic is approximately 4 months from initiation. It is estimated that a pool of 2,000 active donors could yield more than 1 Million doses per week. Further, Oral administration will facilitate rapid deployment, including low-resource settings.

### Example 2: Proof-of-Concept for Epitope Preservation

The ability of clinically relevant immunogenic epitopes to be preserved through sterilization is evaluated by using cultured SARS-CoV-2 virions as a positive control and SARS-CoV-2 negative stool as a negative control. The experimental condition is the combination of SARS-CoV-2 negative stool and cultured SARS-CoV-2 virions. Each condition is subjected to the manufacturing process. Aliquots are saved for activity testing prior to manufacturing and at stages and timepoints within the process.

The activity of the epitopes are measured at each step in the manufacturing process using an ELISA with anti-SARS-CoV-2 antibodies from convalescent serum serving as the capture antibody and anti-human antibodies serving as the detection antibody. Convalescent serum is much more informative than antibodies raised against purified proteins. Not to be limited by theory, antigens bound by convalescent serum are not only immunogenic but also clinically relevant (e.g. neutralizing).

### Example 3: ELISA Assay Preparation and Analysis

Three assays are developed to (1) determine optimal ELISA conditions, (2) test ability of ELISA to detect epitopes in stool matrix and assay loss of signal in heat inactivated material, and (3) refine assay conditions. The assays use the reagents listed in Table 1.

TABLE 1

Assay Reagents

| Item | Note |
|---|---|
| 1. Microtiter Plates | Thermo Fisher Nunc Maxisorp™ flat bottom (Cat. No. 44-2404-21) |
| 2. Coating Buffer | Anhydrous Na2CO3, 1.5 g |
| | Anhydrous NaHCO3, 2.93 g |
| | Distilled water, 1 liter, pH to 9.6 |
| | For an alternative coating buffer Bio-Rad ELISA coating buffer (cat. No. BUF030) is used. |
| | For TGFb-1 ELISA, we made 0.1 M Sodium Carbonate buffer, pH 9.5 (7.13 g NaHCO₃, 1.59 g Na₂CO₃, 1L dH₂O) |
| 3. Blocking Buffer | 1X Phosphate Buffered Saline (PBS) containing 1% w/v BSA For an alternative blocking buffer, use either ELISA BSA |
| | Block (BUF032), ELISA Ultrablock (BUF033), or ELISA Synblock (BUF034) |
| | Sigma-Aldrich A7030 (suitable for ELISA, sandwich ELISA) |
| | For TGFb-1 ELISA, made PBS (10% FBS) |
| 4. Wash Buffer | 1X Phosphate Buffered Saline containing 0.05% v/v Tween®-20 |
| | For an alternative wash buffer use ELISA wash buffer (BUF031) |
| | For TGFb-1 ELISA, also used PBS + 0.05% Tween-20. |
| 5. Recommended Substrates and Stop Solutions | TMB Core+ (BUF062), for use with HRP-conjugated antibodies. |
| | Stop with 0.2 M H2SO4. |
| | For TGFb-1 ELISA, used TMB Substrate reagent set (555214). |
| | Used 2 N H2SO4 (1 M) as stop solution. |
| 6. Antibodies / Enzyme | Anti-CoV-2 polyclonal serum |
| | Anti-human FC (HRP conjugated) e.g Goat anti-human IgG-Biotin or Goat anti human FC IgG Biotin |
| | ThermoFisher 13-4998-83 |
| | Sigma-Aldrich SAB3701279-1MG: 1 mg for $238.00. Est delivery Apr1 |
| | HRP-Streptavidin: Detection conjugate 1 or detection conjugate 2 |
| | ThermoFisher SNN2004 |
| | BioRad 71005 |
| | BioRad STAR5B |

Methodology: To prepare the Self-Sandwich ELISA assay the same polyclonal serum is used as the capture and primary detection antibody. The microtiter plate wells are coated with 100 µl of the anti-SARS-CoV-2 polyclonal serum at a concentration between 1-10 µg/ml in coating buffer. The microtiter plate is then covered and incubated overnight at 4° C. The plate is washed at least 3 times with wash buffer by forcefully filling each well with wash buffer (e.g. approximately 400 µl) with a squirt bottle, multi-channel pipette, manifold dispenser or an auto washer. The liquid is completely removed at each step. Following the last wash, any remaining Wash Buffer is removed by aspirating and inverting and blotting the plate against a clean paper towel. Blocking solution is added at 150 µl per well and the microtiter plate is incubated for 60 minutes at 37° C. The plate is again washed as provided above. An amount of 100 µl of suitably diluted samples are added to the relevant wells (intact virus or S-, N-protein). Ensure that the samples are appropriately diluted are included (dilute samples in wash buffer). Samples or standards are performed in triplicate. Cover with an adhesive strip and incubate for 2 h at RT or overnight at 4° C. Next wash 3 times in wash buffer as above; add 100 µl of Fc block in wash buffer and incubate for 30 min at RT; add 100 µl of the same anti-SARS-CoV-2 serum (used for step 1) (appropriately diluted in wash buffer, see below) and incubate for 1 hour at 37° C. Next wash 3 times in wash buffer; add 100 µl of biotin-conjugated anti-human FC antibody as secondary detection antibody (appropriately diluted in wash buffer, see below) to each well, and incubate for 1 hour at 37° C., followed by washing 3 times in wash buffer. Next 100 µl of HRP-streptavidin conjugate (appropriately diluted in wash buffer) is added to each well and samples incubated for 60 minutes at 37° C. Wash 3 times in wash buffer. Add 100 µl of the appropriate substrate solution to each well (e.g. TMB Core+). Incubate at room temperature (and in the dark if required) for 30 minutes, or until desired color change is attained. Read absorbance values immediately at the appropriate wavelength or add 50 µl of “stop solution”. Gently tap plate to ensure thorough mixing. Measure absorbance within 30 minutes. Determine the optical density (O.D.) of each well within 30 minutes. If using R&D Systems Catalog # DY999 or TMB, set the microtiter plate reader to 450 nm. If wavelength correction is available, set to 540 or 570 nm. If wavelength correction is not available, subtract readings at 540 nm or 570 nm from the readings at 450 nm. This subtraction will correct for optical imperfections in the plates. Readings made directly at 450 nm without correction may be higher and less accurate.

First Assay: In the first assay the dynamic range of viral protein or intact virus detection in buffer is determined using a self-sandwich ELISA (FIG. 1). Briefly, the procedures are as follows. Human anti-SARS-CoV-2 serum is diluted to 0.2, 0.4 and 0.8 µg/ml serum concentrations (in coating buffer) for coating ON at 4C for testing. Whole virus or virus in wash buffer is diluted in ranges of 1 ng/ml, 100 ng/ml, 1 µg/ml for purified protein. SARS-CoV-2 serum available at ATCC has unknown titer (potentially in the 1×10⁵/ml range). The SARS-CoV-2 serum is diluted 1:1, 1:2, and 1:3. Human anti-SARS-CoV-2 serum is also diluted to 50 ng/ml, 100 ng/ml, 200 ng/ml and 400 ng/ml serum concentrations in blocking buffer as a primary detection antibody. The primary antibody is then detected with an anti-human FC biotin conjugate at 50 ng/ml, 100 ng/ml, 200 ng/ml as the secondary detection antibody. The coating, primary, and secondary detection antibodies are added to the microtiter plate as provided in FIG. 2. The experiment is performed in triplets. The optimal assay conditions, including antibody dilutions, are determined and used in the second assay.

Second Assay: This assay is performed to test heat-treated material and determine the signal detectability in stool matrix. The assay consists of the following 6 samples:
1. Negative control 1 is non-pasteurized SARS-CoV-2 negative processed stool.
2. Negative control 2 is pasteurized SARS-CoV-2 negative processed stool.
3. Positive control 1 is non-pasteurized processed stool spiked with SARS-CoV-2 at a range of concentrations (e.g., 50 ng/ml, 100 ng/ml, 200 ng/ml and 400 ng/ml).
4. Positive control 2 is non-pasteurized buffer spiked with SARS-CoV-2 at a concentration determined by the first assay above.
5. Sample type 1 is pasteurized processed stool spiked with SARS-CoV-2 at a range of concentrations (e.g., 50 ng/ml, 100 ng/ml, 200 ng/ml and 400 ng/ml). The processed stool is pasteurized for a total of 10 h with samples taken and tested at 2-3 timepoints.
6. Sample type 2 is SARS-CoV-2 in buffer at a concentration chosen from assay 1 and pasteurized for 10 hrs. The processed stool is pasteurized for a total of 10 h with samples taken and tested at 2-3 timepoints.

The samples are prepared at a 10X concentration for all three concentrations in assay buffer or spiked in at 10X into stool matrix. The samples are incubated for 10 hrs at 60° C. The samples are diluted in 100x wash buffer and both undiluted and diluted samples are tested in the assay. The pasteurized sample in buffer and stool is compared to non-pasteurized samples in buffer and stool.

Third Assay: The third assay is performed to repeat and refine the concentrations based on the results of the second assay. This assay also includes implementing various inactivation temperatures.

### Example 4: Alternative Assay to Anti-SARS-CoV-2 Serum

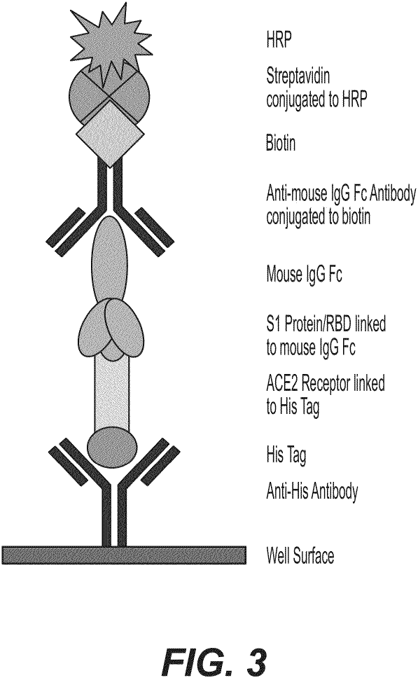

An alternative assay, using the components of Table 2, tests the retention of ACE2-binding activity of S protein after heat-inactivation/pasteurization (FIG. 3). An anti-His tag antibody is bound to a high-protein-affinity plate. The His-tagged human ACE2 receptor protein binds the anti-His antibodies to expose ACE2 interaction site. SARS-CoV-2 S1 protein (full or RBD) on a mouse IgG Fc domain is added to the microtiter well. A protein which retains the capacity to interact with ACE2 binds to the exposed interaction site. An anti-mouse IgG Fc antibody conjugated to biotin is used to detect the SARS-CoV-2 S1 protein. Streptavidin conjugated to HRP and a substrate for HRP are added to produce a colorimetric shift when binding occurs.

TABLE 2

| | | |
|---|---|---|
| Component | Product | Citations |
| Human ACE2 protein, His-Tagged | ACE2 Protein, Human, Recombinant (His Tag) SinoBiological 10108-H08H | |
| SARS-CoV-2 N protein, His-tagged | SARS-CoV-2 (2019-nCoV) Nucleocapsid-His recombinant Protein SinoBiological 40588-V08B | |
| SARS-CoV-2 S1 protein, mouse Fc | SARS-CoV-2 (2019-nCoV) Spike S1-mFc Recombinant Protein SinoBiological 40591-V05H1 | |
| SARS-CoV-2 S1 RBD, mouse Fc | SARS-CoV-2 (2019-nCoV) Spike RBD-mFc Recombinant Protein SinoBiological 40592-V05H | |
| Anti-mouse IgG Fc Antibody, Biotin-conjugated | Goat Anti-Mouse IgG-Fc Secondary Antibody (Biotin) SinoBiological SSA013 | |
| | Goat Anti-Mouse IgG Biotinylated Antibody R&D Systems BAF007 | ELISA: Kim J., et al. Development of a Specific CHIKV-E2 Monoclonal Antibody for Chikungunya Diagnosis. Virologica Sinica 34, 563-571 (2019) (“Kim, J. 2019”); Conlon, T., et al. Germinal centre alloantibody responses are mediated exclusively by indirect-pathway CD4 T follicular helper cells. J. of Immunol. 188(6): 2643-2652 (2012) (“Conlon, T. 2012”); Young Hwang, H., et al. High specific inhibition of C1q Globular-head binding to human IgG. Mol Immunol. 45(9): 2570-2580 (2008) (“Young 2008”); Thebeau, L., et al. Mechanism of Reduced T-Cell Effector Functions and Class- |
| | | Switched Antibody Responses to Herpes Simplex Virus Type 2 in the Absence of B7 Costimulation. J Virol. 77(4): 2426-2435 (2003) (“Thebeau 2003”) |
| | Goat anti-Mouse IgG Fc Secondary Antibody, Biotin ThermoFisher (Invitrogen) A16088 | |
| | Goat Anti-Mouse IgG Fc (Biotin) Abcam ab97263 | immunohistochemistry (IHC): Kim, J. 2019 |
| | Anti-Mouse IgG (Fc specific)-Biotin antibody produced in rabbit Sigma-Aldrich, SAB3701025 | ELISA: Kim, J. 2019 |
| | Anti-Mouse IgG (Fc specific)-Biotin antibody produced in goat Sigma-Aldrich, B9904 | ELISA: Kim, J. 2019; Conlon, T. 2012; Young 2008; Thebeau 2003; and Briney, B. Tailored immunogens direct affinity maturation toward HIV neutralizing antibodies. Cell. 166(6): 1459-1470 (2016) (“Briney 2016”) Also citations for immunofluorescence (IF), IHC, western blot (WB) |
| | Rabbit F(ab′)2 Anti-Mouse IgG:Biotin Bio-Rad STAR11B | ELISA: Kim, J. 2019; Conlon, T. 2012; Young 2008 ELISpot: Kim, J. 2019 |
| Anti-mouse, IgG Fc Antibody, HRP conj ugated | Goat Anti-Mouse IgG Antibody, HRP conjugate Sigma-Aldrich (note: validated for ELISA) | |
| | Goat Anti-Mouse IgG FC (HRP) AbCam AB97265 (Note: Validated for ELISA) | ELISA: Kim, J. 2019 |
| Anti-His Antibody (Rabbit) | His tag Polyclonal Antibody ThermoFisher (ProteinTech) 10001-0-AP (note: not validated for ELISA) | WB: Kim, J. 2019; Conlon, T. 2012; Young 2008 |
| | 6x-His Tag Polyclonal Antibody ThermoFisher (Invitrogen) PA1-983B (note: not validated for ELISA) | WB: Kim, J. 2019; Conlon, T. 2012; Young 2008 |
| | 6x-His Tag Recombinant Polyclonal Antibody (21HCLC) ThermoFisher (Invitrogen) 710286 (note: not validated for ELISA) | IHC: Kim, J. 2019 |
| | 6x-His Tag Recombinant Rabbit Monoclonal Antibody (RM146) ThermoFisher (Invitrogen) MA5-33032 (note: not validated for ELISA) | |
| | His-tag Antibody, pAb, Rabbit GenScript, A00174 (note: validated for ELISA) | ELISA: Kim, J. 2019 WB: Kim, J. 2019 |
| | Anti-6-His antibody produced in rabbit Sigma-Aldrich SAB4301134 (note: not validated for ELISA) | WB: Kim, J. 2019 |
| | Anti-6X His tag® antibody Abcam ab137839 (note: not validated for ELISA) | WB: Kim, J. 2019; Conlon, T. 2012 |
| | 6X His Epitope Tag Antibody Rockland 600-401-382 (note: validated for ELISA) | WB: Kim, J. 2019; Conlon, T. 2012 |
| | His Tag Antibody (3D5) Novus Biologicals NBP2-52637 (note: validated for ELISA) | |
| | His Tag Antibody (RMH01) Novus Biologicals NBP2-61478 (note: validated for ELISA) | |
| | His-Tag Antibody #2365 Cell Signalling Technologies 2365 (note: not validated for ELISA) | |
| Anti-His Antibody (Goat) | His Tag Antibody Novus Biologicals NBP1-25939 (note: validated for ELISA) | Immunocytochemistry (ICC)/IF: Kim, J. 2019 |
| | Histidine Tag antibody BioRad AHP1656 (note: validated for ELISA) | |
| | Anti-6X His tag® antibody Abcam ab9136 (note: validated for ELISA) | WB: Kim, J. 2019; Conlon, T. 2012 |
| Anti-His Antibody (Rat) | His Tag Antibody (3D5) Novus Biologicals NBP2-81023 (note: validated for ELISA) | |
| Anti-His Antibody (Human) | His Tag Antibody (3D5) Novus Biologicals NBP2-81021 (note: validated for ELISA) | |
| | Anti-His tag Antibody SinoBiological 105327-MM02T | ELISA: Kim, J. 2019 WB/IF: Kim, J. 2019 |
| Anti-His Antibody (Mouse) | (note: not validated for ELISA,; monoclonal mouse IgG) | |
| | Anti-His tag Antibody (HRP) SinoBiological 105327-MM02T-H (note: validated for ELISA; monoclonal mouse IgG) | |
| | His Tag Antibody R&D Systems MAB050 (note: monoclonal mouse IgG) | ELISA and WB: Kim, J. 2019; Conlon, T. 2012; Young 2008; Thebeau 2003; Briney 2016; Heuberger, D., et al. Thrombin cleaves and activates the protease-activated receptor 2 dependent on thrombomodulin co-receptor availability. Thrombosis Research. 177: 91-101 (2019) (“Heuberger 2019”); Martinez-Murillo, P., et al. Particulate array of well-ordered HIV clade C Env trimers elicits neutralizing antibodies that display a unique V2 cap approach. Immunity. 46(5): 804-817 (2017) (“Martinez-Murillo 2017”); and Feng, Y., et al. Thermostability of Well-Ordered HIV Spikes Correlates with the Elicitation of Autologous Tier 2 Neutralizing Antibodies. PLoSPathog. 12(8): e1005767 (2016) (“Feng 2016”) |
| | Anti-His6 from mouse IgG1 Sigma-Aldrich/Roche 11922416001 | WB: Kim, J. 2019 |
| | 6x-His Tag Monoclonal Antibody (HIS.H8) ThermoFisher (Invitrogen) MA1-21315 (note: mouse IgG2b, multiple ELISA pubs) | ELISA: Kim, J. 2019; Conlon, T. 2012; Young 2008; Thebeau 2003; Briney 2016 Also citations for IHC, ICC, WB, IF |
| | 6x-His Tag Monoclonal Antibody (4A12E4) ThermoFisher (Invitrogen) 37-2900 (note: mouse IgG1, 2 ELISA pubs) | ELISA: Kim, J. 2019; Conlon, T. 2012 Also citations for ICC, WB, IF |
| | Histidine Tag antibody (AD1.1.10) Bio-Rad MCA1396 (note: monoclonal mouse IgG) | ELISA: Kim, J. 2019; Conlon, T. 2012; Young 2008; Thebeau 2003; Briney 2016 |
| | Anti-6X His tag® antibody [HIS.H8] Abcam ab18184 (note: monoclonal mouse IgG) | ELISA: Kim, J. 2019; Conlon, T. 2012 Citations for WB, IF |

### Example 5: Antibodies From COVID-19 Survivors Reveal Clinically Important Viral Epitopes and Demonstrate Their Stability in a Vaccine

Use of convalescent serum for epitope detection provides insights into clinically relevant viral antigens, as these epitopes generated antibodies in patients that successfully cleared the virus. The receptor binding domain of the Spike protein of SARS-CoV-2 facilitates high affinity interactions with the ACE2 receptor on respiratory epithelial cells and is critical to infectivity of SARS-CoV-2. Several studies indicate that neutralizing antibodies in COVID-19 patients target the spike protein RBD and epitopes of the S1 and S2 domains in order to inhibit viral binding to respiratory epithelial cells.

Purified SARS-CoV-2 protein (S1, RBD, N) is immobilized on a microplate with or without prior pasteurization and viral protein is then detected using convalescent serum collected from COVID-19 survivors and labeled anti-human antibodies.

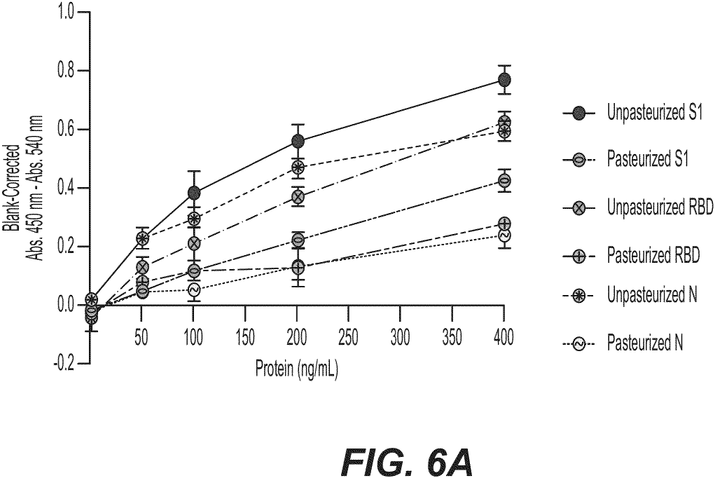

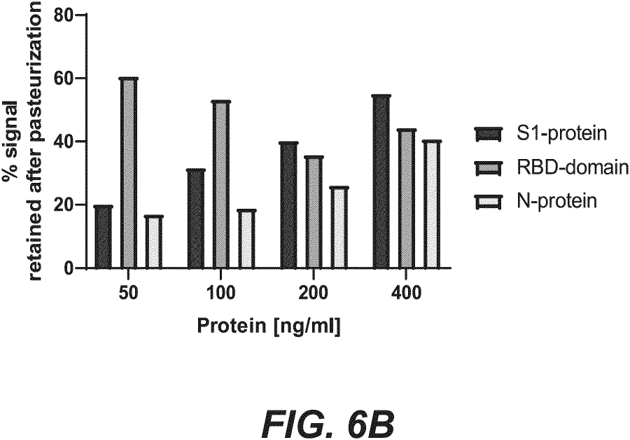

FIG. 6A shows the observed signal detected across five concentrations of protein, demonstrating that SARS-CoV-2 epitopes can be quantified with COVID-19 convalescent serum. FIG. 6B shows the same dataset as FIG. 6A, with the measured difference between unpasteurized and pasteurized proteins at each concentration computed as the percentage of epitopes retained after pasteurization. This shows that 20-60% of detectable epitopes are heat-stable, yielding ample substrates to drive immune response.

In total, the results show that RBD, a likely target for neutralizing antibodies, has the greatest thermal stability among the three proteins evaluated.

### Example 6: Treatment of Type 1 Diabetes (T1D) by Administering a Non-Viable Bacterial Preparation

Type 1 diabetes (T1D), also known as juvenile diabetes or insulin-dependent diabetes, is an autoimmune disease that targets pancreatic islet beta cells, causing the pancreas to produce little or no insulin. Without insulin, blood sugar cannot reach the cells and builds up in the blood stream. High blood sugar damages the body and causes many of the symptoms and complications of diabetes. Standard therapy for managing T1D includes the administration of insulin to the patient on a regular basis to manage blood sugar levels. Recently, it has been uncovered that gut microbiome might be a hub of T1D-triggering factors (Zheng, P., et al. Diabetes Metab Res Rev.; 34(7): e3043 (2018)).

A T1D patient is administered a non-viable bacterial preparation (i.e., FMT preparation pasteurized to inactivate cells). The administration of the non-viable bacterial preparation causes a reduction of T1D symptoms.

### Example 7: Treatment of Non-Small Cell Lung Cancer (NSCLC) by Co-Administering The Non-Viable Bacterial Preparation Along With Pembrolizumab

Despite the efficacy of immune checkpoint inhibitors (ICIs), only 20-30% of treated patients present long term benefits (Botticelli, A., et al., J Transl Med, 18(1):49 (2020)). Recent studies have found that the gut microbiome can impact the response to immunotherapy in cancer patients, although the underlying mechanisms are poorly understood. Pembrolizumab, an anti-PD1 monoclonal antibody and immune checkpoint inhibitor, has been approved for the treatment of NSCLC (Lim, SH., et al., Expert Opin Biol Ther 2016;16(3):397-406).

A patient with NSCLC is administered a non-viable bacteria preparation along with Pembrolizumab. Treatment with the non-viable bacteria preparation increases the immunotherapy response in a cancer patient.

### Example 8: Treatment of a Peanut Allergy by Co-Administering the Non-Viable Bacterial Preparation and a Peanut Allergen

Peanut allergy is a type of food allergy to peanuts caused by a type I hypersensitivity reaction of the immune system in susceptible individuals (CMAJ 2003 May 13; 168(10):1279-85). This allergy is one of the most severe food allergies due to its prevalence, persistency, and potential severity (Clin Exp Allergy 1995 Jun;25(6):493-502). Allergen immunotherapy is a form of long-term treatment that decreases allergy symptoms via the repetitive exposure of the patient to the immunogenic epitope of an antigen (e.g., an allergen) to cause an immune response (Allergy Shots (Immunotherapy), American Academy of Allergy Asthma & Immunology, Sept. 28, 2020, www.aaaai.org).

A patient with peanut allergies is administered a non-viable bacteria preparation with a peanut allergen to treat their peanut allergy. Treatment with the non-viable preparation facilitates or enhances the efficacy of anti-allergy therapy because the non-viable bacteria preparation shifts the ratio of immune cells in favor of cells that suppress an immune reaction to the allergen.

1-38. (canceled)

39. A method of stimulating the immune system in a subject in need thereof, the method comprising administering to the subject a pharmaceutical composition comprising an adjuvant comprising a fecal bacterial preparation, wherein the fecal bacterial preparation comprises fecal bacteria from a stool of a human donor.

40. The method of claim 39, wherein the fecal bacteria are not viable.

41. The method of claim 39, wherein the subject is further administered an immunomodulator.

42. The method of claim 41, wherein the immunomodulator stimulates the immune system of the subject.

43. The method of claim 41, wherein the immunomodulator comprises an antigen of a pathogen.

44. The method of claim 43, wherein the fecal bacterial preparation comprises the antigen of the pathogen.

45. The method of claim 43, wherein the pathogen is a virus.

46. The method of claim 43, wherein the pathogen is a bacterial pathogen.

47. The method of claim 41, wherein the immunomodulator is administered in a pharmaceutical composition that does not comprise the fecal bacterial preparation.

48. The method of claim 41, wherein the immunomodulator is an immunotherapy.

49. The method of claim 48, wherein the immunotherapy comprises an anti-cancer immunotherapy for treatment of a cancer.

50. The method of claim 49, wherein the anti-cancer immunotherapy is selected from the group consisting of dendritic cell-based pump-priming or vaccination, T-cell adoptive transfer checkpoint inhibitor therapy, and a combination thereof.

51. The method of claim 50, wherein the anti-cancer immunotherapy is checkpoint inhibitor therapy.

52. The method of claim 51, wherein the checkpoint inhibitor therapy comprises administering to the subject a compound that recognizes a checkpoint molecule selected from the group consisting of PD-1, PD-L1, PD-L2, ICOS, ICOSL, and CTLA-4.

53. The method of claim 51, wherein the immunotherapy comprises administering to the subject a compound selected from the group consisting of Pembrolizumab, Nivolumab, or Ipilimumab.

54. The method of claim 49, wherein the cancer is lung cancer.

55. The method of claim 54, wherein the lung cancer is non-small cell lung cancer.

56. The method of claim 48, wherein the immunotherapy is an allergen immunotherapy.

57-61. (canceled)

62. The method of claim 39, wherein the pharmaceutical composition comprises a capsule.

63. The method of claim 39, wherein the fecal bacteria comprise uncultured fecal bacteria, and the uncultured fecal bacteria are released from a capsule in the subject’s small intestine.

64-70. (canceled)
